(19) United States
(12) Patent Application Publication
(10) Pub. No.: US 2013/0324533 A1
(43) Pub. Date: Dec. 5, 2013

(54) METHOD OF DETECTING RESISTANCE TO CANCER THERAPY

(71) Applicants: Yijun Ruan, Singapore (SG); Sin Tiong Ong, Singapore (SG); King-Pan Ng, Singapore (SG); Charles Thuan Heng Chuah, Singapore (SG); Axel Maximilian Hillmer, Singapore (SG); Wen Chun Juan, Singapore (SG)

(72) Inventors: Yijun Ruan, Singapore (SG); Sin Tiong Ong, Singapore (SG); King-Pan Ng, Singapore (SG); Charles Thuan Heng Chuah, Singapore (SG); Axel Maximilian Hillmer, Singapore (SG); Wen Chun Juan, Singapore (SG)

(73) Assignees: NATIONAL UNIVERSITY OF SINGAPORE, Singapore (SG); AGENCY FOR SCIENCE, TECHNOLOGY AND RESEARCH, Singapore (SG); SINGAPORE HEALTH SERVICES PTE LTD, Singapore (SG)

(21) Appl. No.: 13/993,745

(22) PCT Filed: Dec. 14, 2011
(86) PCT No.: PCT/SG11/00437

§ 371 Date: Jun. 13, 2013

(30) Foreign Application Priority Data
Dec. 14, 2010 (SG) 201009324-3

(51) Int. Cl. C12Q 1/68

(52) U.S. Cl. CPC C12Q 1/6886
USPC 514/235.8; 435/6.11; 514/275; 514/252.19

(57) ABSTRACT

We describe a polymorphic variant of a BIM {BCL2L11) gene which comprises, in 5′ to 3′ order, the nucleotide sequence set out in SEQ ID NO: 5 followed immediately by the nucleotide sequence set out in SEQ ID NO: 7. The BIM polymorphic variant may be characterised by lacking the nucleotide sequence set out in SEQ ID NO: 6. It may be used to detect BCR-ABL-independent TKI-resistance (resistance to treatment with tyrosine kinase inhibitors) for chronic myelogenous leukaemia, c-KIT/PDGFR-independent TKI-resistance for gastrointestinal stromal tumours (GIST), EGFR-independent TKI-resistance for non-small cell lung cancer (NSCLC) or JAK2-independent TKI-resistance for a myeloproliferative disorder, in an individual comprising such a polymorphism.

### FIELD

The present invention relates to the fields of medicine, cell biology, molecular biology and genetics.

### BACKGROUND

Chronic myelogenous leukemia (CML) is a cancer of haematopoietic stem cells, and is caused by the presence of the oncogenic fusion gene, termed BCR-AB L, that is found in all patients with CML.

BCR-ABL encodes for a constitutively active tyrosine kinase that mediates the increased survival and proliferation of CML cells when compared to their normal counterparts. Effective treatment for CML exists in the form of a class of drugs that inhibits the kinase activity of BCR-AB L, which are commonly called tyrosine kinase inhibitors (TKI).

However, a small proportion of patients will exhibit primary resistance to TKIs and not respond, while others will have an initial response but over time, develop secondary resistance to these drugs, an event that is often associated with transformation to blast crisis (BC) CM L, and shorter survival. In about 60% of patients with TKI-resistance (ref AI), BCR-ABL kinase activity is found to be reactivated, either through mutations in the BCR-ABL gene that render TKIs less able to bind to BCR-AB L, or via amplification and overexpression of the ‘wild type’ BCR-ABL gene or protein.

In the remaining 40% of cases, the cause of the TKI-resistance is unknown. The discovery of mechanisms which mediate TKI-resistance in the absence of reactivation of BCR-AB L, also termed BCR-ABL-independent TKI-resistance, are critically important to determine strategies to overcome TKI-resistance, and better manage patients with CML.

Thus, therapy with BCR-ABL inhibitors has resulted in high rates of disease control in the majority of patients with chronic phase chronic myelogenous leukemia (CML). However, a significant proportion of patients still fail to achieve optimal responses,¹ and almost all patients with late-stage disease succumb to their illness.²

Attempts utilizing clinically-driven risk scores to stratify patients for clinical outcome have had limited success,^{3,4} in part because such scores do not take into account the molecular features of the disease or patient-specific genetic factors that contribute to clinical outcomes. Indeed, almost nothing is known of host genetic factors that are predictive of response in chronic phase CML.⁵⁻⁷

As a result, current clinical recommendations are to treat all patients with the same starting dose of imatinib, and to monitor for benchmark clinical responses at regular intervals. These include normalization of peripheral blood counts, and degree of cytogenetic and molecular responses at three- to six-monthly intervals.⁵ Only upon failure to attain benchmarks is therapy altered. As such decision points, options include increasing the dose of imatinib, switching to more potent tyrosine kinase inhibitors, as well as preparation for high-dose chemotherapy and transplantation.

Ideally, it would be possible to tailor therapy according to both the leukemia and the patient so as to achieve the most rapid response as well as avoid the emergence of drug-resistance or disease progression. For these reasons, reliable markers for predicting response and guiding therapy are needed. Recent work has relied on array-based expression profiling to provide insights into genetic factors associated with drug resistance and disease progression in CML.⁸ However, such approaches are limited by their inherent bias as well as their inability to define the underlying genetic events that contribute to these expression profiles.

### SUMMARY

According to a 1^{st} aspect of the present invention, we provide a method of predicting whether an individual susceptible to or suffering from cancer or a myeloproliferative disorder is likely to develop resistance to treatment with a tyrosine kinase inhibitor.

The method may comprise determining whether the individual has a polymorphic variant of a BIM (BCL2L11) gene. The polymorphic variant or polymorphism may comprise, in 5′ to 3′ order, the nucleotide sequence set out in SEQ ID NO: 5 followed immediately by the nucleotide sequence set out in SEQ ID NO. The polymorphic variant of the BIM (BCL2L11) gene may lack the nucleotide sequence set out in SEQ ID NO: 6.

The method may comprise detecting the presence of a nucleic acid amplification product comprising a sequence set out in SEQ ID NO: 1 as an indicator of the individual having the polymorphic variant of the BIM (BCL2L11) gene. The method may comprise, alternatively or in addition, detecting the presence of a nucleic acid amplification product comprising a sequence set out in SEQ ID NO: 2 as an indicator of the individual lacking the polymorphic variant of the BIM (BCL2L11) gene.

The method may comprises nucleic acid amplification. The method may utilise a nucleotide sequence as set out in SEQ ID NO: 3. It may utilise a nucleotide sequence set out in SEQ ID NO: 4. It may utilise a combination of such sequences. The combination may comprise as a primer set.

The method may be such that if the individual is determined to have the polymorphic variant of the BIM (BCL2L11) gene, then the individual is likely to develop resistance to treatment with a tyrosine kinase inhibitor. Alternatively, or in addition, the method may be such that if the individual is determined not to have the polymorphic variant of the BIM (BCL2L11) gene, then the individual is less likely to develop resistance to treatment with a tyrosine kinase inhibitor.

There is provided, according to a 2^{nd} aspect of the present invention, a method of choosing a therapy, for an individual with cancer or a myeloproliferative disorder. The method may comprise determining whether a patient is likely to develop resistance to treatment with a tyrosine kinase inhibitor by a method as set out above. The method may be such that where the individual is determined as being likely to develop such resistance, choosing a therapy.

The therapy may comprise more frequent monitoring of the patient. The therapy may comprise more frequent blood tests. The therapy may comprise more frequent bone marrow tests. The therapy may comprise bone marrow transplantation. The therapy may comprise administration of a more potent tyrosine kinase inhibitor (TKI). The tyrosine kinase inhibitor may comprise nilotinib or dasatinib, or both. The therapy may comprise administration of a BH3-mimetic. The BH3-mimetic may comprise ABT-263. The BH3-mimetic may be administered in combination with a TKI. The therapy may comprise increasing the dose of a tyrosine kinase inhibitor. The tyrosine kinase inhibitor may comprise imatinib. The dose may be increased beyond the standard dose of 400 mg/day to 600 or 800 mg/day. The therapy may comprise treatment with a drug that inhibits the pro-survival effect of the BCL2 group of proteins.

We provide, according to a 3^{rd} aspect of the present invention, a method of determining the likelihood of success of a particular therapy on an individual with cancer or a myeloproliferative disorder. Such a method may comprise comparing the therapy with the therapy determined by a method set out above.

The cancer or myeloproliferative disorder may comprise chronic myelogenous leukaemia (CML). The resistance to treatment with a tyrosine kinase inhibitor may comprise BCR-ABL-independent TKI-resistance. The resistance to treatment with a tyrosine kinase inhibitor may comprise resistance to a tyrosine kinase inhibitor. The tyrosine kinase inhibitor may comprise imatinib.

The cancer or myeloproliferative disorder may comprise gastrointestinal stromal tumour (GIST). The resistance to treatment with a tyrosine kinase inhibitor may comprise c-KIT/PDGFR-independent TKI-resistance. The resistance to treatment with a tyrosine kinase inhibitor may comprise resistance to imatinib.

The cancer or myeloproliferative disorder may comprise non-small cell lung cancer (NSCLC). The resistance to treatment with a tyrosine kinase inhibitor may comprise EGFR-independent TKI-resistance. The resistance to treatment with a tyrosine kinase inhibitor may comprise resistance to any one or more or a combination of erlotinib, gefitinib, sunitinib, nilotinib and sorafenib.

The cancer or myeloproliferative disorder may comprise a myeloproliferative disorder such as selected from the group consisting of: polycythaemia vera, essential thrombocythaemia, and primary myelofibrosis. The resistance to treatment with a tyrosine kinase inhibitor may comprise JAK2-independent TKI-resistance. The resistance to treatment with a tyrosine kinase inhibitor may comprise resistance to JAK inhibitors.

The cancer or myeloproliferative disorder may be selected from the group consisting of: haematologic malignancies, chronic lymphocytic leukaemia, acute lymphoblastic leukaemia, acute myeloid leukaemia, multiple myeloma, myeloproliferative disorders (including polycythaemia vera, essential thrombocythaemia, and primary myelofibrosis), solid tumours, small cell lung cancer, breast cancer, colorectal cancer, ovarian cancer, melanoma and neuroblastoma.

As a 4^{th} aspect of the present invention, there is provided a polymorphic variant of a BIM (BCL2L11) gene which comprises, in 5′ to 3′ order, the nucleotide sequence set out in SEQ ID NO: 5 followed immediately by the nucleotide sequence set out in SEQ ID NO: 7.

We provide, according to a 5^{th} aspect of the present invention, a polymorphic variant of a BIM (BCL2L11) gene characterised by lacking the nucleotide sequence set out in SEQ ID NO: 6.

The present invention, in a 6^{th} aspect, provides a nucleotide sequence as set out in SEQ ID NO: 1 or SEQ ID NO: 2. Such a nucleotide sequence may be obtainable from a BIM polymorphic variant as described above. It may be obtainable by nucleic acid amplification.

The polymorphic variant of a BIM (BCL2L11) gene or the nucleotide sequence set out above may be associated with resistance to treatment with tyrosine kinase inhibitors for chronic myelogenous leukaemia in the absence of BCR-ABL reactivation (BCR-ABL-independent TKI-resistance). They may be associated with resistance to treatment with tyrosine kinase inhibitors for gastrointestinal stromal tumours (GIST) in the absence of c-KIT/PDGFR reactivation (c-KIT/PDGFR-independent TKI-resistance). They may be associated with resistance to treatment with tyrosine kinase inhibitors for non-small cell lung cancer (NSCLC) in the absence of EGFR reactivation (EGFR-independent TKI-resistance). They may be associated with resistance to treatment with tyrosine kinase inhibitors for a myeloproliferative disorder in the absence of JAK2 reactivation (JAK2-independent TKI-resistance).

In a 7^{th} aspect of the present invention, there is provided a nucleotide sequence as set out in SEQ ID NO: 3. We further provide a nucleotide sequence as set out in SEQ ID NO: 4. We also provide a combination of such nucleotide sequences. This may be provided as a primer set.

According to an 8^{th} aspect of the present invention, we provide a method of detecting the presence or absence of a BIM (BCL2L11) polymorphism set out above in an individual. The method may comprise detecting a nucleic acid amplification product comprising a sequence set out in SEQ ID NO: 1. The method may comprise detecting a sequence set out in SEQ ID NO: 2. The detection may make use of a primer set out above.

We provide, according to a 9^{th} aspect of the invention, a method of treatment of a patient suffering from cancer or a myeloproliferative disorder. The method may comprise determining whether the cancer or myeloproliferative disorder is a BCR-ABL-independent TKI-resistant CML cancer. The method may comprise determining whether the cancer or myeloproliferative disorder is a c-KIT/PDGFR-independent TKI-resistant GIST cancer. The method may comprise determining whether the cancer or myeloproliferative disorder is an EGFR-independent TKI-resistant NSCLC cancer. The method may comprise determining whether the cancer or myeloproliferative disorder is a JAK2-independent TKI-resistant myeloproliferative disorder. The method may be as set out above. The method may further comprise treating the patient by performing a step selected from (a) to (g) as set out in the 2^{nd} aspect of the invention.

Such a method may comprise detecting a nucleic acid amplification product comprising a sequence set out in SEQ ID NO: 1. This may be done by use of a primer set as described above.

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; D. M. J. Lilley and J. E. Dahlberg, 1992, Methods of Enzymology: DNA Structure Part A: Synthesis and Physical Analysis of DNA Methods in Enzymology, Academic Press; Using Antibodies: A Laboratory Manual: Portable Protocol No. I by Edward Harlow, David Lane, Ed Harlow (1999, Cold Spring Harbor Laboratory Press, ISBN 0-87969-544-7); Antibodies: A Laboratory Manual by Ed Harlow (Editor), David Lane (Editor) (1988, Cold Spring Harbor Laboratory Press, ISBN 0-87969-314-2), 1855. Handbook of Drug Screening, edited by Ramakrishna Seethala, Prabhavathi B. Fernandes (2001, New York, N.Y., Marcel Dekker, ISBN 0-8247-0562-9); and Lab Ref: A Handbook of Recipes, Reagents, and Other Reference Tools for Use at the Bench, Edited Jane Roskams and Linda Rodgers, 2002, Cold Spring Harbor Laboratory, ISBN 0-87969-630-3. Each of these general texts is herein incorporated by reference.

### BRIEF DESCRIPTION OF THE FIGURES

FIG. 1 shows a view from the UCSC Genome Browser of Human genome (hg18) chr2:111,593,210-111,644,581 for UCSC Genes (top) and RefSeq Genes (GeneBank, bottom). The BIM gene and the deletion characterising the polymorphism is marked.

FIG. 2 shows a DNA-PET analysis of six CML genomes.

FIG. 2A summarizes the clinico-pathologic features of the patient samples and CML cell line used for DNA-PET analysis.

FIG. 2B shows the karyo-genomic maps of six CML genomes which were generated from DNA-PET data using Circos,²⁹ and depicted as circular plots. SVs which matched those identified in at least one of 32 normal genomes were filtered out (these SVs correspond to those in Table E4). The different categories of structural variations (SV) are arranged in concentric layers as indicated in the key. The asterisks (*) indicate the presence of the BCR-ABL1/ABL1-BCR translocation.

FIG. 3 shows somatic structural variations associated with blast transformation.

FIG. 3A shows the Genome browser view of the novel rearrangement in the EVI1 locus in P098. Genes known to be in this locus [University of California Santa Cruz (UCSC)] (Rhead et al. 2010) are shown in red and blue (top). Red tracks reflect the fragment coverage of concordant PETs. Dark red and pink horizontal arrowheads represent mapping regions (anchors) of discordant PETs indicating the presence of the insertion.

FIG. 3B shows the expression levels of EVI1 as measured by quantitative real-time PCR. EVI1 levels are shown for two blast phase samples (P022 and P098) and four chronic phase samples (P308, P355, P490, and P500), normalized against expression of β-actin.

FIG. 3C shows the florescence in situ hybridization analysis of the EVI1 rearrangement using custom-made probes for chromosome 3 (RP11-137H17, green) and chromosome 8 (RP11-828L6, red; RP11-159N7, yellow) as depicted in FIG. 3A. The EVI1 rearrangement is absent in the normal control but can be seen as red-green-yellow fusion signal in the P098 sample.

FIG. 4 shows a polymorphic 2903 bp deletion in intron 2 of BIM is present in all three resistant samples.

FIG. 4A shows the detection of the BIM deletion by DNA-PET in three of three samples from patients with resistance to tyrosine kinase inhibitors (P308, P022, and P098), but not in samples from patients or cell lines sensitive to tyrosine kinase inhibitors (P145, P440, and K562). Chr2:111580000.111650000 DNA-PET data of five clinical CML samples and K562 are shown in the Genome Browser. Dark red and pink horizontal arrow heads connected by green lines represent mapping regions (anchors) of discordant PETs indicating the presence of deletions. The vertical dashed lines depict the deleted region.

FIG. 4B shows that the region is conserved in mammals as demonstrated by the UCSC Genome Browser ‘28-Way Cons’ Track for the deletion (chr2:111, 599, 666 . . . 111, 602, 568) with the degree of conservation on the y-axis.

FIG. 4C shows the validation of the deletion by PCR using genomic DNA samples from the five patients and K562 cells. PCR products with a size of 4.2 Kb correspond to the non-deletion allele, while PCR products with a size of 1.3 Kb correspond to the deletion allele. M, 1 Kb marker (Fermentas).

FIG. 4D depicts the identical breakpoints found in all three deletion-containing samples by Sanger sequencing of PCR products. Deleted sequences are indicated in blue.

FIG. 5 shows functional effects of the BIM polymorphism.

FIG. 5A depicts the genomic organization of BIM showing exons for the major BIM isoforms including BIMEL, BIML, and BIMS, as well as for BIMγ (the only isoform known to contain exon 3).³⁰⁻³² The polymorphic deletion between exon 2 and 3 is highlighted by the red line. The exons containing the start codon (Start), dynein binding domain (DBD), BH3 domain (BH3), and stop codon/polyadenlyation signal sequence (Stop/PolyA) are also highlighted. The diagram is not drawn to scale.

FIG. 5B shows the expression levels of exon-specific transcripts of BI M, as measured by quantitative real-time PCR in 23 CML patient samples [n=11 without the deletion (WT) and n=12 with the deletion (carriers)]. The levels of the various transcripts containing exons E2A, E3 or E4 are expressed as normalized ratios relative to exon 2A or β-actin. The mean and standard error of the mean are represented by the red lines and bars. Statistical significance (p) is calculated by Wilcoxon Ranks sums test.

FIG. 5C shows the PCR reaction products from a collection of East-Asian and non-East-Asian CML cell lines performed to detect the deletion, using the method described in Example 2.

FIG. 5D shows the ratio of exon 3- to exon 4-containing transcripts in CML cell lines with and without the deletion.

FIG. 5E is a Western blot showing levels of BIMEL in cell lysates obtained from cell lines, with and without the deletion, following treatment with 1 μM imatinib for 24 hours.

FIG. 5F shows the growth curve of CML cells lines with and without the deletion cultured with 1 μM imatinib.

FIG. 5G shows the apoptotic activity of imatinib, dasatinib and ABT-737 against KCL22 and KYO-1 cells. Cells were treated with 2 μM imatinib, 50 nM dasatinib or 2.5 μM ABT-737 for 48 hrs, and the percentage of dead cells was determined by flow cytometery.

FIG. 6 shows association of the BIM polymorphism with clinical resistance to tyrosine kinase inhibitors. Patients seen at two South East-Asian referral centers with either chronic or accelerated phase CML were categorized according their sensitivity or resistance to tyrosine kinase inhibitors (TKI) by European LeukemiaNet criteria, as well as the presence or absence of mutations in BCR-ABL that are known to confer resistance. The incidence of the BIM polymorphism in each of these groups was then determined. P values were calculated using a two-tailed Fisher's exact test.

FIG. 6A shows the frequency of the BIM deletion in patients with TKI-resistance with BCR-ABL mutations versus TKI-resistance in the absence of BCR-ABL mutations.

FIG. 6B shows the frequency of the BIM deletion in patients with TKI-sensitive disease versus TKI-resistance in the absence of BCR-ABL mutations.

FIG. 6C shows the frequency of the BIM deletion in patients with TKI-sensitive disease compared to all patients with TKI-resistance.

FIG. 7 shows copy number information deduced from the cPET tag counts. Chromosomes are arranged on horizontal axis in alternating green and black as indicated on the bottom. Copy number values are represented on the Y-axis with smoothened window values indicated in red. Sample IDs are shown in the top left corners of plots. Note the different y-axis scale for K562.

FIG. 8 shows genomic background of BIM deletion in East Asians.

FIG. 8A. Seventy-four East Asian HapMap phase I individuals were genotyped for the intronic deletion in BIM and deletion genotypes were correlated with SNP genotypes using HaploView software (Barrett et al. 2005). LD based haplotype block is shown with SNPs in genomic order from left to right. Haplotype frequencies are show in gray on the right. Marker #49 represents the BIM deletion with A=no deletion and C=deletion. The deletion is on the background of the blue haplotype which is apart from the deletion identical with the most frequent red haplotype. Haplotype tagging SNPs are marked with an arrow head.

FIG. 8B, IDs (Name) of tagging SNPs in A (#) are shown with their heterozygosity frequency (ObsHET), minor allele frequency (MAF) and alleles.

FIG. 9. DNA paired-end tag (PET) sequencing method. Genomic DNA was hydrosheared, EcoP15I recognition sites were methylated and EcoP15I CAP adaptors were ligated to the ends of DNA fragments. The methylated DNA constructs were separated on agarose gel and 9 Kb sized fragments were selected for ligation resulting in circularized products where 5′ (R3, dark red) and 3′ (F3, pink) ends of 9 Kb fragments were connected by an internal biotinylated adaptor with two flanking non-methylated EcoP15I CAP adaptors. Constructs were digested by methylation sensitive EcoP151 to release 5′ and 3′ PET constructs. Sequencing adaptors were ligated to the PET constructs, which were then amplified by PCR and sequenced by the Applied Biosystems SOLiD system. Resulting PETs were mapped to the human reference sequence hg18.

FIG. 10. Identification of structural variations (SVs) by dPET clusters. ‘Interpretation’ indicates the genomic structure of the sequenced genome deduced from the mapping pattern of the dPET clusters to the human reference sequence (‘Mapping to reference’). Dark red arrows represent 5′ anchor regions and pink arrows represent 3′ anchor regions. Gray, blue, and red horizontal lines represent chromosomal segments. Red arrows indicate orientation of chromosomal segments.

FIG. 11. Reconstruction of cytogenetically predicted isochromosome 17q. DNA-PET cluster of size 2 connects chromosome 17 position 17,595,066 minus strand with chromosome 17 position 28,282,853 plus strand. Top, Chromosome 17 ideogram is shown with break point locations indicated by red vertical lines. Dark red and pink arrows symbolise mapping positions and orientations of PETs. Middle, Genome browser view is shown for break point regions which correspond to red lines on top. Red track represents coverage by concordant mapping PETs; genes are shown in green; dark red and pink arrows indicate mapping positions and orientations of PETs. Bottom, reconstruction of isochromosome 17q based on DNA-PET data. Arrows indicate direction of increasing genomic coordinates.

FIG. 12. Somatic structural variations associated with blast transformation.

FIG. 12A Shown are the genome structures of BCR and ABL1 genes and location of translocation break points. Exons are indicated by boxes, introns are represented by feathered lines indicating direction of transcription (+strand, left to right). Locations of break points are indicated by red vertical lines with respective sample IDs.

FIG. 12B The ratio of the number of paired-end reads (cluster size normalized by coverage) connecting BCR with ABL1 vs the reciprocal event correlates with disease progression. Genomic regions of BCR on chromosome 22 and ABL1 on chromosome 9 are shown on horizontal axis with genes shown in green and blue. Copy number estimates are represented by purple tracks. Pink and dark red arrows indicate connectivity between BCR and ABL1. The number of PETs of the BCR-ABL1 and ABL1-BCR translocations are shown in blue and grey, respectively. Dashed vertical lines indicate location of break points.

FIG. 12C FISH validation of BCR-ABL1 translocations identified by DNA-PET are shown.

FIG. 13. Expression levels of exon-specific transcripts of BIM. Expression levels are measured by quantitative real-time PCR in 7 cell lines generated from normal individuals who are not affected by CML [n=3 without the deletion (WT), n=4 with the deletion (Carriers)]. The levels of the various transcripts containing exons E2A, E3 or E4 are expressed as normalized ratios relative to exon 2A or β-actin. Expression for the one homozygous carrier identified is highlighted in green. Statistical significance (p) is calculated by two-tailed Wilcoxon Ranks sums test (red) and two-tailed t-test (black).

FIGS. 14A to 14D are diagrams showing a 2903 bp deletion polymorphism in intron 2 of BIM is present in TKI resistant CML patient samples.

FIG. 14A. A Genome Browser view of the DNA-PET data encompassing Chr2:111, 580,000 . . . 111,650,000 from the five clinical CML samples and K562 cells is shown. Detection of the BIM deletion polymorphism by DNA-PET in three of three samples from patients with resistance to imatinib (P308, P022, and P098), but not in samples from patients or cell lines sensitive to imatinib (P145, P440, and K562). Red tracks represent the number of the sequenced concordant PETs which map to the region (coverage). Burgundy and pink horizontal arrowheads connected by green lines represent mapping regions of discordant PETs, and indicate the presence of a deletion. The vertical dashed lines depict the deleted region.

FIG. 14B. Schematic depicting the intronic BIM deletion polymorphism and flanking sequences. The breakpoints were identified by Sanger sequencing of PCR products. Deleted sequences are highlighted in blue. Human reference sequence coordinates are based on NCBI Build 36.

FIG. 14C. Agarose gel showing the PCR products from the five patient samples and K562 cells using primers that flanked the deletion. PCR products with a size of 4,226 bps and 1,323 bps correspond to the no-deletion and deletion alleles respectively. The presence of both the 4,226 and 1,323 bp products indicate that the individual is heterozygous for the deletion polymorphism.

FIG. 14D. Table showing the frequencies of the deletion polymorphism in different ethnic populations. The samples were genotyped by PCR and analyzed on agarose gels as shown in a.

FIGS. 15A to 15J are diagrams showing effects of the deletion polymorphism on BIM gene function.

FIG. 15A. Genomic organization of BIM showing exons for the major BIM isoforms including BIMEL, BIML, and BIMS, as well as for BIMγ, which lacks the BH3 domain¹⁶. The deletion polymorphism between exon 2 and 3 is highlighted by the red line. The exons containing the start codon (Start), dynein binding domain (DBD), BH3 domain (BH3), and stop codon/polyadenylation signal sequence (Stop/PolyA) are also highlighted. The diagram is not drawn to scale.

FIG. 15B. Schematic of the two minigene constructs that were transfected into either K562 or KCL22 CML cells, and that served as a readout for measuring splicing to exon 3/4. The exon 3/4 ratios were obtained by Q-PCR using specific primers for the U-E3 and U-E4 transcripts, normalized to the adenovirus-specific exonic sequence (U).

FIG. 15C. Histograms of the increased ratio of E3 to E4 transcripts in the Non-deletion minigene construct compared to the Deletion minigene construct in K562 cells (left-hand graph), and in KCL22 cells (right-hand graph) (mean+/−standard error of the mean, *p=0.0002, **p=0.012).

FIG. 15D. Expression levels of exon-specific transcripts of BI M, as measured by quantitative real-time PCR (Q-PCR) in 23 CML patient samples [n=11 without the deletion (WT) and n=12 with the deletion (carriers)]. The levels of the various transcripts containing exons E2A, E3 or E4 are expressed as normalized ratios relative to E2A (for E3 and E4) or b-actin (for E2A [total BIM transcripts]). The mean and standard error of the mean are represented by the red lines and bars. Expression levels for the one homozygous carrier are highlighted in green. Statistical significance was determined using the Wilcoxon Rank Sum test.

FIG. 15E. Agarose gel of the PCR products, using the method described in FIG. 14, identifying the polymorphism in a collection of East-Asian and non-East-Asian CML cell lines.

FIG. 15F. Ratio of E3- to E4-containing transcripts in CML cell lines with (KCL22) and without (K562 and KYO-1) the deletion polymorphism (mean+/−standard error of the mean, *p=0.016, **p=0.011).

FIG. 15G. Expression levels of exon 4-specific transcripts of BIM (normalized to β-actin), as measured by Q-PCR in cell lines, with and without the deletion polymorphism, following treatment with 1 mM imatinib for 24 hours (mean+/−standard error of the mean, *p=0.01, **p=0.004 with respect to imatinib-treated KCL22 cells).

FIG. 15H. Western blot showing levels of BIMEL (contains exon 4 and the BH3 domain) and CrkL in cell lysates from cell lines treated as in FIG. 15G. In all three cell lines, imatinib exposure resulted in the dephosphorylation of CrkL, indicating the inhibition of BCR-ABL kinase activity.

FIG. 15I. Western blot showing caspase 3 cleavage in cells treated as in FIG. 15G.

FIG. 15J. Western blot of cell lines treated with imatinib, with or without ABT-737, and assayed for caspase 3 cleavage.

FIGS. 16A to 16H are diagrams showing de novo generation and analysis of CML cell lines bearing the BIM deletion polymorphism.

FIG. 16A. Schematic representation of the strategy of using zinc finger nucleases (ZFN) to introduce the BIM deletion polymorphism into the genome of the K562 CML cell line.

FIG. 16B. PCR products using the primers depicted in FIG. 16A to detect the polymorphism in genome-edited K562 cell lines, with KCL22 cells as a control. Clones that were negative (K562-BIM^{intron+/+}), as well as heterozygous (K562-BIM^{intron+/-}) and homozygous (K562-BIM^{intron-/-}) for the deletion polymorphism were isolated.

FIG. 16C. Exon 3/exon 4 transcript ratio as measured by Q-PCR in K562-BIM^{intron+/+}, K562-BIM^{intron+/-} and K562-BIM^{intron-/-} cells (mean+/−standard error of the mean, *p=0.002, **p=0.0001).

FIG. 16D. Expression of E4-containing transcripts following imatinib exposure in K562-BIM^{intron+/+}, K562-BIM^{intron+/-} and K562-BIM^{intron-/-} cells (mean+/−standard error of the mean, *p=0.015, **p=0.001).

FIG. 16E. Western blot of cell lysates from K562-BIM^{intron+/+}, K562-BIM^{intron+/-} and K562-BIM^{intron-/-} cells following treatment with increasing concentrations of imatinib. Western blots were probed with antibody to BIMEL, cleaved caspase 3, PAR P, CrkL, and β-actin.

FIG. 16F. Densitometry histograms of the findings in e for BIMEL (**p=0.0086, ***p=0.00016) and cleaved PARP (*P=0.018, **P=0.0084) normalized to β-actin and uncleaved PARP respectively (mean+/−standard error of the mean).

FIG. 16G. Relative apoptotic cell death using an ELISA-based assay to detect mono- and oligo-nucleosomes in the cytoplasmic fraction of apoptotic cells (mean+/−standard error of the mean, **p=0.006, ***p=0.00021).

FIG. 16H. Western blot demonstrating the effect of adding the BH3 mimetic drug, ABT-737, to imatinib with respect to apoptotic signaling K562-BIM^{intron+/+}, K562-BIM^{intron+/-} and K562-BIM^{intron-/-} cells.

FIG. 17. DNA Paired-End Tag (PET) Sequencing Method. Genomic DNA was hydrosheared, EcoP15I recognition sites were methylated and EcoP15I CAP adaptors were ligated to the ends of DNA fragments. The methylated DNA constructs were separated on agarose gel and 5, 7, and 9 Kb sized fragments, respectively, were selected for ligation resulting in circularized products where 5′ (R3, dark red) and 3′ (F3, pink) ends of 5, 7, or 9 Kb fragments were connected by an internal biotinylated adaptor with two flanking non-methylated EcoP15I CAP adaptors. Constructs were digested by methylation sensitive EcoP151 to release 5′ and 3′ PET constructs. Sequencing adaptors were ligated to the PET constructs, which were then amplified by PCR and sequenced by the Applied Biosystems SOLiD system. Resulting PETs were mapped to the human reference sequence NCBI build 36 (figure adapted from)¹.

FIG. 18. DNA-PET Analysis of Six CML Genomes. The karyo-genomic maps of six CML genomes which were generated from DNA-PET data using Circos¹⁸, and depicted as circular plots. SVs which matched those identified in at least one of 31 normal genomes were filtered out (Tables H3 to H 5). The different categories of structural variations (SV) are arranged in concentric layers as indicated in the key. The asterisks (*) indicate the presence of the BCR-ABL/ABL-BCR translocation. The chromosome 9/22 translocation lost its balanced character in P098 due to a 3 Mb deletion of the reciprocal ABL/BCR fusion on der9 and in K562 due to complex rearrangements. The translocation found in P440 is likely to have been an insertion, and was also found in the paired sample from the same patient (P145) by PC R, but not by DNA-PET (see footnote for Table H5).

FIGS. 19A to 19C. BCR-ABL rearrangement in Six CML Genomes.

FIG. 19A shows the genome structures of the BCR and ABL1 genes, as well as the location of translocation breakpoints. Exons are indicated by black boxes while introns are represented by feathered lines indicating the direction of transcription (+ strand, left to right). Locations of the breakpoints are indicated by red vertical lines along with the respective sample IDs.

FIG. 19B shows that the ratio of the number of paired-end reads (cluster size) connecting BCR with ABL1 (blue), as well as the reciprocal event connecting ABL1 with BCR (grey), correlates with disease stage. Genomic regions of BCR on chromosome 22 and ABL1 on chromosome 9 are shown on the horizontal axis with BCR and ABL1 in green. Copy number estimates are represented by purple tracks. Pink and burgundy arrows indicate connectivity between BCR and ABL1. The DNA-PET signals corrected for the sample specific sequencing depth (coverage; see Table H1) of the BCR-ABL and ABL-BCR translocations are shown in blue and gray, respectively. Dashed vertical lines indicate location of breakpoints. ‘MMR’=major molecular remission.

FIG. 19C shows FISH validation of the BCR-ABL translocations identified by DNA-PET. FISH probes for BCR and ABL1 are labeled green and red respectively. In accordance with the DNA-PET results, P145 shows the balanced translocation event (two fusions) and one normal copy of each (1G1R), P440 shows no translocation event between BCR and ABL1 loci (2R2G), P022 shows the balanced translocation and one additional fusion corresponding to an extra copy of BCR-ABL (three fusions) and one normal copy of each (1G1R), P098 shows the fusion between BCR-ABL (one fusion) and the remaining signal for the BCR probe (1G) corresponding to the ABL1 deletion on chromosome 9 and one normal copy of each (1G1R).

FIG. 20. Exon 3 of BIM Contains a Stop Codon and a Polyadenylation Signal. The nucleotide sequence and the derived amino acid sequence of BIMγ are shown with residue and base number indicated on the right. The nucleotide sequence for exon 3 is highlighted in blue, and the stop codon (TGA) within exon 3 is highlighted in red. The polyadenylation signal (AATAAA) in exon 3 is underlined.

FIG. 21. BIML and BIMS are More Potent Inducers of Apoptosis Compared to BIMγ. BIM L, BIMS and BIMγ cDNAs were cloned into pcDNA3-FLAG3 plasmids (generous gifts from Koji Itahana). 5 μg of plasmids were nucleofected into KCL22 cells and the amount of apoptotic cells were measured using Annexin V-FITC/7-AAD staining 24 hours post-nucleofection. All data shown are means of three independent experiments (+/−standard error of the mean).

FIGS. 22A to 22C. siRNA-Mediated Knockdown of BIMγ Does not Sensitize KCL22 Cells to Imatinib.

FIG. 22A shows the apoptotic activity of imatinib on KCL22 cells upon BIMγ knockdown. Cells were transfected with siRNA for 24 hrs (U=untransfected control), and treated with 2 μM imatinib for another 48 hrs, and the percentage of apoptotic cells determined by flow cytometry.

FIG. 22B shows the expression levels of E3-containing transcripts of BIM in KCL22 cells transfected with control or E3-specific siRNA, as measured by quantitative real-time PCR. Statistical significance was determined by Student's t-test.

FIG. 22C shows the ratio of E3- to E4-containing transcripts in KCL22 cells transfected with control or E3-specific siRNA, compared to that of K562 and KYO1. All data shown are means of three independent experiments (+/−standard error of the mean).

FIG. 23. Functional Effects of the BIM Deletion Polymorphism on BIM Gene Splicing in Lymphoblastoid Cell Lines. The expression levels of exon-specific transcripts of BI M, as measured by quantitative real-time PCR (Q-PCR) in lymphoblastoid cell lines from seven normal HapMap individuals [three without the deletion (WT), and four with the deletion (Carriers)]. The levels of the various transcripts containing exons E2A, E3 or E4 are expressed as normalized ratios relative to E2A (for E3 and E4) or β-actin (for E2A). One of the carriers was homozygous (green diamond).

FIG. 24. Comparison of dPET Clusters Identified by DNA-PET in Samples Obtained During Chronic Phase and After Remission from the Same Patient. dPET clusters which passed ‘Exclusion of low confidence clusters’ were compared between patient sample P145 and P440. Clusters were categorized in two groups: clusters which were identified in both samples (shared clusters) and clusters which were observed in only one of the two samples (unique cluster). Within these groups, the fraction of clusters with sizes 2 to 50 are shown.

FIGS. 25A to 25F are diagrams showing HCC2279, an NSCLC cell line carrying the BIM deletion polymorphism, is intrinsically resistant to gefitinib.

FIG. 25A. An agarose gel of the PCR products identifying the polymorphism in HCC2279 cells, using the method previously described¹³.

FIG. 25B. Ratio of E3- to E4-containing transcripts in NSCLC cell lines with (HCC2279) and without (PC9) the deletion polymorphism (mean+/−standard error of the mean).

FIG. 25C. Expression levels of exon 4-containing BIM transcripts (normalized to β-actin), as measured by Q-PCR in PC9 and HCC2279, following treatment with control (DMSO) or 0.5 μM gefitinib for 24 h (mean+/−standard error of the mean, *p=0.0025 with respect to gefitinib-treated PC9 cells).

FIG. 25D. Western blot showing levels of BIMEL (contains exon 4 and the BH3 domain) and PARP in cell lysates from cell lines treated with control or 0.5 μM gefitinib for 24 h.

FIG. 25E. Western blot showing levels of phospho-EGFR (p-EGFR), PAR P, and caspase 3 cleavage in cells treated for 24 h with 0.5 μM gefitinib, 2.5 μM ABT-737, or both.

FIG. 25F. Cell viability of PC9 and HCC2279 treated for 48 h with 0.5 μM gefitinib, 2.5 μM ABT-737, or both. Cell viability was determined by trypan blue exclusion, and plotted as a percentage of the no-drug control. (mean+/−standard error of the mean, *p=0.00004 with respect to gefitinib- and ABT-737-treated PC9 cells).

FIG. 26 is a diagram showing that the BIM deletion polymorphism predicts poorer progression free survival in patients with EGFR-mutant non-small cell lung cancers treated with EGFR TKI therapy. The presence or absence of the BIM deletion polymorphism was determined in 141 NSCLC patients from Singapore and Japan who were known to have activating mutations in EGFR, and who received TKI therapy. Progression free survival for each group was estimated using the Kaplan-Meier method.

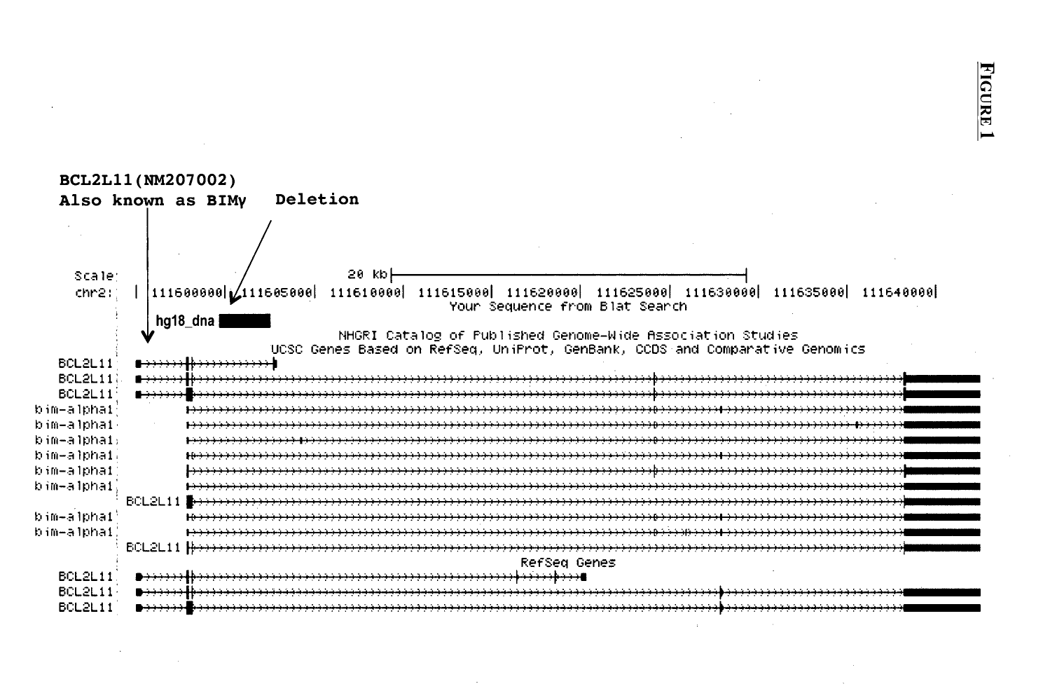

### SEQUENCE LISTING

SEQ ID NO: 1 is the sequence of PCR fragment amplified using BIM_del_F and BIM_del_R primers from a BIM gene with the deletion described (length 1,323 bp).

SEQ ID NO: 2 is the sequence of PCR fragment using BIM_del_F and BIM_del_R primers from a BIM wild type gene (length 4,226 bp).

SEQ ID NO: 3 is the sequence of the Bim_del_F forward primer (+chr2:111,599,051 . . . 111,599,070, hg18)

SEQ ID NO: 4 is the sequence of the Bim_del_R reverse primer (−chr2:111,603,257 . . . 111,603,276, hg18)

SEQ ID NO: 5 is the sequence of the 1000 bp flanking sequence upstream: chr2:111,598,666-111,599,665 of the Deletion in BIM: chr2:111,599,666-111,602,568 (hg18)

SEQ ID NO: 6 is the sequence of the deletion in BIM: chr2:111,599,666-111,602,568 (hg18)

SEQ ID NO:7 is the sequence of the 1000 bp flanking sequence downstream: chr2:111,602,569-111,603,568 of the deletion in BIM: chr2:111,599,666-111,602,568 (hg18)

### DETAILED DESCRIPTION

This invention describes a novel polymorphism that we have found in East-Asian populations in the BIM gene. This polymorphism is associated with the development of drug-resistance in patients with chronic myelogenous leukaemia (CML).

Patients who have CML and who harbour this polymorphism are more likely to develop resistance to the standard therapy for CML than those who do not. Testing for this polymorphism is useful as a biomarker for the prediction of drug-resistance, and the early identification of patients who may benefit from alternative and/or more aggressive therapies. Furthermore, because the BIM gene is an important mediator of therapy-induced cell death in other cancer types, our invention may also be relevant to a wide range of human cancers in East-Asian populations.

In order to discover mechanisms of TKI-resistance, we applied a novel technology termed genomic paired-end ditag or DNA-PET (ref A2, ref A3), coupled with high-throughput sequencing to interrogate the genome of CML cells from patients with or without drug-resistance. In doing so, we uncovered a previously unknown deletion in the BIM gene that was associated with the development of BCR-ABL-independent TKI-resistance. Patients with this deletion are four to five times (17.4 vs 3.9%) more likely to have BCR-ABL-independent TKI-resistance than those without the deletion (p=0.04, two-tailed Fisher's exact test). In addition, we have also been able to correlate the presence or absence of the BIM deletion to BCR-ABL-independent TKI-resistance in CML cell lines from patients, suggesting a direct mechanistic role for BIM deletions in TKI-resistance. Since it is known that upregulation of proapoptotic isoforms of the BIM protein is required for TKI-mediated CML cell death (refs A4-A7), we hypothesize that BIM deletions impair the expression of proapoptotic BIM.

### BIM Polymorphism

We describe isolated polynucleotides comprising one or more BIM polymorphic nucleic acid molecules. These, as well as the corresponding polypeptides, are variously described in this document as a “polymorphic variant of a BIM (BCL2L11) gene” or simply a “BIM polymorphism”.

The isolated polynucleotides may be used in a variety of diagnostic methods. Isolated polymorphic BIM nucleic acid molecules as described here may be used in one or more of the following methods: a) screening assays; b) predictive medicine (e.g., diagnostic assays, prognostics assays, monitoring clinical trials, and pharmacogenetics); and c) methods of treatment (e.g., therapeutic and prophylactic).

BIM genes are known in the art, as described elsewhere in this document. The source of BIM gene can be any mammalian BIM gene. In general, for diagnostic assays, the animal source of the BIM gene will be the same species as the animal whose nucleic acid is being tested.

An isolated polymorphic BIM nucleic acid molecule comprises one or more BIM polymorphisms. For example, the BIM polymorphism may comprise, in 5′ to 3′ order, the nucleotide sequence set out in SEQ ID NO: 5 followed immediately by the nucleotide sequence set out in SEQ ID NO: 7. The BIM polymorphic variant may be characterised by lacking the nucleotide sequence set out in SEQ ID NO: 6.

For some uses, e.g., in screening assays, BIM polymorphic nucleic acid molecules will be of at least about 15 nucleotides (nt), at least about 18 nt, at least about 20 nt, or at least about 25 nt in length, and often at least about 50 nt. Such small DNA fragments are useful as primers for polymerase chain reaction (PCR), hybridization screening, etc. Larger polynucleotide fragments, e.g., at least about 50 nt, at least about 100 nt, at least about 200 nt, at least about 300 nt, at least about 500 nt, at least about 1000 nt, at least about 1500 nt, up to the entire coding region, or up to the entire coding region plus up to about 1000 nt 5′ and/or up to about 1000 nt 3′ flanking sequences from a BIM gene, are useful for production of the encoded polypeptide, promoter motifs, etc. For use in amplification reactions, such as PC R, a pair of primers will be used. The exact composition of primer sequences is not critical, but for most applications the primers will hybridize to the subject sequence under stringent conditions, as known in the art.

When used as a probe, an isolated polymorphic BIM nucleic acid molecule may comprise non-BIM nucleotide sequences, as long as the additional non-BIM nucleotide sequences do not interfere with the detection assay. A probe may comprise an isolated polymorphic BIM sequence, and any number of non-BIM nucleotide sequences, e.g., from about 1 bp to about 1 kb or more.

For screening purposes, hybridization probes of the polymorphic sequences may be used where both forms are present, either in separate reactions, spatially separated on a solid phase matrix, or labeled such that they can be distinguished from each other. Assays may utilize nucleic acids that hybridize to one or more of the described polymorphisms.

Isolated polymorphic BIM nucleic acid molecules described here may be coupled (e.g., chemically conjugated), directly or indirectly (e.g., through a linker molecule) to a solid substrate. Solid substrates may be any known in the art including, but not limited to, beads, e.g., polystyrene beads; chips, e.g., glass, silica, and the like; plastic surfaces, e.g., polystyrene, polycarbonate plastic multi-well plates; and the like.

Isolated polymorphic BIM nucleic acid molecules can be obtained by chemical or biochemical synthesis, by recombinant DNA techniques, or by isolating the nucleic acids from a biological source, or a combination of any of the foregoing. For example, the nucleic acid may be synthesized using solid phase synthesis techniques, as are known in the art. Oligonucleotide synthesis is also described in Edge et al. (1981) Nature 292:756; Duckworth et al. (1981) Nucleic Acids Res. 9:1691 and Beaucage and Caruthers (1981) Tet. Letters 22:1859. Following preparation of the nucleic acid, the nucleic acid is then ligated to other members of the expression system to produce an expression cassette or system comprising a nucleic acid encoding the subject product in operational combination with transcriptional initiation and termination regions, which provide for expression of the nucleic acid into the subject polypeptide products under suitable conditions.

Additional BIM gene polymorphisms may be identified using any of a variety of methods known in the art, including, but not limited to SSC P, denaturing HPLC, and sequencing. SSCP may be used to identify additional BIM gene polymorphisms. In general, PCR primers and restriction enzymes are chosen so as to generate products in a size range of from about 25 bp to about 500 bp, or from about 100 bp to about 250 bp, or any intermediate or overlapping range therein.

Polymorphic BIM Polypeptides

We further describe isolated polymorphic BIM polypeptides. Isolated polymorphic BIM polypeptides may be useful in assays to screen for agents that modify a biological activity of a BIM polypeptide.

The term “polymorphic BIM polypeptide” encompasses an amino acid sequence encoded by an open reading frame (ORF) of a known BIM polynucleotide, including the full-length native polypeptide and fragments thereof, particularly biologically active fragments and/or fragments corresponding to functional domains, e.g. a region or domain having biological activity, etc.; antigenic fragments thereof, and including fusions of the subject polypeptides to other proteins or parts thereof. The amino acid sequences of BIM polypeptides have been disclosed. See e.g. Moore et al., supra. A polymorphism in a BIM polypeptide is generally defined relative to a reference sequence.

As used herein, “polymorphic BIM polypeptide” refers to an amino acid sequence of a recombinant or non-recombinant polypeptide having an amino acid sequence of i) a native polymorphic BIM polypeptide, ii) a fragment of a polymorphic BIM polypeptide, iii) polypeptide analogs of a polymorphic BIM polypeptide, iv) variants of a polymorphic BIM polypeptide; v) an immunologically active fragment of a polymorphic BIM polypeptide; and vi) fusion proteins comprising a polymorphic BIM polypeptide. Polymorphic BIM polypeptides can be obtained from a biological sample, or from any source whether natural, synthetic, semi-synthetic or recombinant.

The term “polymorphic BIM polypeptide” encompasses a polypeptide comprising from at least about 5 amino acids, at least about 10 amino acids, at least about 15 amino acids, at least about 25 amino acids, at least about 50 amino acids, at least about 75 amino acids, at least about 100 amino acids, at least about 200 amino acids, at least about 300 amino acids, at least about 400 amino acids, or up to the entire polypeptide of a polymorphic BIM polypeptide. In some embodiments, a polymorphic BIM polypeptide exhibits biological activity, e.g., the polypeptide causes proliferation of B-cells and production of immunoglobulin in an in vitro assay. Other assays for BIM biological activity are known in the art and can be used to determine whether a polymorphic BIM polypeptide exhibits biological activity and, if desired, to quantitate BIM biological activity. BIM biological assays are described in various publications, e.g., Moore et al., supra.

Polymorphic BIM polypeptides may be part of a fusion protein. Suitable fusion partners (e.g., a non-BIM polypeptide, or “heterologous polypeptide”) include, but are not limited to, a heterologous polypeptide that provides for immunological recognition, e.g., an epitope tag; a heterologous polypeptide that provides for a detectable signal, e.g., a green fluorescent protein (GFP), β-galactosidase, and the like; a heterologous polypeptide that provides for a catalytic function; and a heterologous polypeptide that facilitates entry into a cell. The fusion partner can be coupled in-frame to the N-terminus, the C-terminus, or both of the polymorphic BIM polypeptide, using standard methods for synthesis of polypeptides, or using recombinant methods.

Polymorphic BIM polypeptides can be obtained by any known method, or a combination of such methods, including isolation from natural sources; production by chemical synthesis; and production by standard recombinant techniques.

Polymorphic BIM polypeptides can be isolated from a biological source, using affinity chromatography, e.g., using antibodies specific for a BIM polypeptide are immobilized on a solid support. The polypeptides may be expressed in prokaryotes or eukaryotes in accordance with conventional ways, depending upon the purpose for expression. For large scale production of the protein, a unicellular organism, such as E. coli, B. subtilis, S. cerevisiae, insect cells in combination with baculovirus vectors, or cells of a higher organism such as vertebrates, particularly mammals, e.g. COS 7 cells, CHO cells, HEK293 cells, and the like, may be used as the expression host cells. In some situations, it is desirable to express the gene in eukaryotic cells, where the protein will benefit from native folding and post-translational modifications. The polypeptide can then be isolated from cell culture supernatant or from cell lysates using affinity chromatography methods or anion exchange/size exclusion chromatography methods, as described above.

With the availability of the protein or fragments thereof in large amounts, by employing an expression host, the protein may be isolated and purified in accordance with conventional ways. A lysate may be prepared of the expression host and the lysate purified using HPLC, exclusion chromatography, gel electrophoresis, affinity chromatography, or other purification technique.

### Detection of BIM Polymorphisms

A number of different methods are commonly used to analyze DNA for polymorphisms or mutations, for example, the BIM polymorphism described here.

Thus, detection of a BIM polymorphism in a polynucleotide sample derived from an individual can be accomplished by any means known in the art, including, but not limited to, amplification of a sequence with specific primers; determination of the nucleotide sequence of the polynucleotide sample; hybridization analysis; single strand conformational polymorphism analysis; denaturing gradient gel electrophoresis; mismatch cleavage detection; and the like. Detection of the BIM polymorphism can also be accomplished by detecting an alteration in the level of a mRNA transcript of a BIM gene; aberrant modification of a BIM gene, e.g., an aberrant methylation pattern; the presence of a non-wild-type splicing pattern of BIM mRNA; an alteration in the level of BIM polypeptide; and/or an alteration in BIM polypeptide biological activity.

Samples

Polynucleotide samples derived from (e.g., obtained from) an individual are obtained from a biological sample taken from the individual. Any biological sample that comprises a polynucleotide from the individual is suitable for use. The biological sample may be processed so as to isolate the polynucleotide. Alternatively, whole cells or other biological samples may be used without isolation of the polynucleotides contained therein.

Nucleic Acid Amplification

Detection of a BIM polymorphism by analyzing a polynucleotide sample can be conducted in a number of ways. A test nucleic acid sample can-be amplified with primers which amplify a sequence region known to comprise a BIM polymorphism(s). Genomic DNA or mRNA can be used directly. Alternatively, the region of interest can be cloned into a suitable vector and grown in sufficient quantity for analysis.

The nucleic acid may be amplified by conventional techniques, such as a polymerase chain reaction (PCR), ligase chain reaction (LCR), etc to provide sufficient amounts for analysis. The use of the polymerase chain reaction is described in a variety of publications, including, e.g., “PCR Protocols (Methods in Molecular Biology)” (2000) J. M. S. Bartlett and D. Stirling, eds, Humana Press; and “PCR Applications: Protocols for Functional Genomics” (1999) Innis, Gelfand, and Sninsky, eds., Academic Press.

A detectable label may be included in an amplification reaction. Suitable labels include fluorochromes, e.g. fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein (JOE),6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA), radioactive labels, e.g. ³²P, ³⁵S, ³H; etc. The label may be a two stage system, where the amplified DNA is conjugated to biotin, haptens, etc. having a high affinity binding partner, e.g. avidin, specific antibodies, etc., where the binding partner is conjugated to a detectable label. The label may be conjugated to one or both of the primers. Alternatively, the pool of nucleotides used in the amplification is labeled, so as to incorporate the label into the amplification product.

Once the region comprising a BIM polymorphism has been amplified, the BIM polymorphism can be detected in the PCR product by nucleotide sequencing, by SSCP analysis, or any other method known in the art.

Nucleic Acid Sequencing

The most definitive method is to sequence the DNA or transcribed mRNA (if applicable) to determine the actual base sequence (Maxam and Gilbert, 1977; Sanger et al., 1977).

The sample nucleic acid may be sequenced by a dideoxy chain termination method or other well-known methods. Genomic DNA or mRNA may be used directly. If mRNA is used, a cDNA copy may first be made. If desired, the sample nucleic acid can be amplified using a PCR. A variety of sequencing reactions known in the art can be used to directly sequence the BIM gene, or a portion thereof in which a specific polymorphism is known to occur, and detect polymorphisms by comparing the sequence of the sample nucleic acid with a reference polynucleotide that contains a BIM polymorphism. Any of a variety of automated sequencing procedures can be used. See, e.g., WO 94/16101; Cohen et al. (1996) Adv. Chromatography 36:127-162.

Although such a method is the most definitive it is also the most expensive and time consuming method.

Restriction Mapping Analysis (RFLP Analysis)

Specific DNA sequences in an individual, for example, a gene encoding the polymorphic variant of a BIM (BCL2L11) gene, may undergo many different changes, such as deletion of a sequence of DNA, insertion of a sequence that was duplicated, inversion of a sequence, or conversion of a single nucleotide to another. Changes in a specific DNA sequence may be traced by using restriction enzymes that recognize specific DNA sequences of 4-6 nucleotides.

Restriction mapping analysis may therefore also be used in analyzing DNA for polymorphisms. If one is looking for a known polymorphism at a site which will change the recognition site for a restriction enzyme it is possible simply to digest DNA with this restriction enzyme and analyze the fragments on a gel or with a Southern blot to determine the presence or absence of the polymorphism. This type of analysis is also useful for determining the presence or absence of gross insertions or deletions. Hybridization with allele specific oligonucleotides is yet another method for determining the presence of known polymorphisms.

Restriction enzymes cut (digest) the DNA at their specific recognized sequence, resulting in one million or so pieces. When a difference exists that changes a sequence recognized by a restriction enzyme to one not recognized, the piece of DNA produced by cutting the region are of a different size. The various possible fragment sizes from a given region therefore depend on the precise sequence of DNA in the region.

Variation in the fragments produced is termed “restriction fragment length polymorphism” (RFLP). The different sized-fragments reflecting different variant DNA sequences may be visualized by separating the digested DNA according to its size. Fractionation may be performed by various means, such as gel or capillary electrophoresis, particularly acrylamide or agarose gels.

For example, DNA fragments may be on an agarose gel and visualizing the individual fragments by annealing to a radioactively labeled DNA “probe”. Each individual may carry two different forms of the specific sequence. When the two homologues carry the same form of the polymorphism, one band is seen. More than two forms of a polymorphism may exist for a specific DNA marker in the population, but in one family just four forms are possible; two from each parent. Each child inherits one form of the polymorphism from each parent. Thus, the origin of each chromosome region may be traced (maternal or paternal origin).

The aforementioned techniques are well known in the art. Detailed description of these techniques can be found in a variety of publications, including, e.g., “Laboratory Methods for the Detection of Mutations and Polymorphisms in DNA” (1997) G. R. Taylor, ed., CRC Press, and references cited therein.

Reverse Translation Polymerase Chain Reaction (RT-PCR)

Alternatively or in addition, RT-PCR may be carried out. PCR may thus also be used to determine whether a polymorphism is present by using a primer that is specific for the polymorphism.

Such methods may comprise the steps of collecting from an individual a biological sample comprising the individual's genetic material as template, optionally isolating template nucleic acid (genomic DNA, mRNA, or both) from the biological sample, contacting the template nucleic acid sample with one or more primers that specifically hybridize with a BIM polymorphic nucleic acid molecule under conditions such that hybridization and amplification of the template nucleic acid molecules in the sample occurs, and detecting the presence, absence, and/or relative amount of an amplification product and comparing tie length to a control sample. Observation of an amplification product of the expected size is an indication that the BIM polymorphism contained within the BIM polymorphic primer is present in the test nucleic acid sample.

Parameters such as hybridization conditions, BIM polymorphic primer length, and position of the polymorphism within the BIM polymorphic primer may be chosen such that hybridization will not occur unless a polymorphism present in the primer(s) is also present in the sample nucleic acid. Those of ordinary skill in the art are well aware of how to select and vary such parameters. See, e.g., Saiki et al. (1986) Nature 324:163; and Saiki et al (1989) Proc. Natl. Acad. Sci. USA 86:6230.

Hybridisation Analysis

Hybridization with the variant sequence may also be used to determine the presence of a BIM polymorphism.

Hybridization analysis can be carried out in a number of different ways, including, but not limited to Southern blots, Northern blots, dot blots, microarrays, etc. The hybridization pattern of a control and variant sequence to an array of oligonucleotide probes immobilized on a solid support, as described in U.S. Pat. No. 5,445,934, or in WO 95/35505, may also be used as a means of detecting the presence of variant sequences.

Identification of a polymorphism in a nucleic acid sample can be performed by hybridizing a sample and control nucleic acids to high density arrays containing hundreds or thousands of oligonucleotide probes. Cronin et al. (1996) Human Mutation 7:244-255; and Kozal et al. (1996) Nature Med. 2:753-759.

Mass Spectrometry

The use of mass spectrometry to determine the presence of polymorphisms within known genes is disclosed in U.S. Pat. No. 5,869,242.

Oligonucleotide Ligation

Alternatively, various methods are known in the art that utilize oligonucleotide ligation as a means of detecting polymorphisms. See, e.g., Riley et al. (1990) Nucleic Acids Res. 18:2887-2890; and Delahunty et al. (1996) Am. J. Hum. Genet. 58:1239-1246.

Single Strand Conformational Polymorphism (SSCP) Analysis

Single strand conformational polymorphism (SSCP) analysis is a rapid and efficacious method for detecting polymorphisms (Dean et al., Cell 61:863, 1990; Glavac and Dean, Hum. Mutation 2:404, 1993; Poduslo et al., Am. J. Hum. Genet. 49:106, 1992). In SSC P, abnormal strand motility on a gel is associated with mutational events in the gene.

Denaturing gradient gel electrophoresis (DGGE), mismatch cleavage detection and heteroduplex analysis in gel matrices may also be used to detect polymorphisms.

Restriction Endonuclease Fingerprinting (REF)

Restriction endonuclease fingerprinting (REF) may also be conducted, optionally after RT-PCR is carried out. REF is a modification of the single-strand conformation polymorphism (SSCP) method, and enables efficient detection of sequence alterations in DNA fragments up to 2 kb in length (Liu and Sommer, 1995).

Genetic analysis of polymorphisms is disclosed in detail in U.S. Pat. Nos. 5,552,28, 5,654,13, 5,670,33, 5,807,67, 5,858,66, 5,691,15, 5,922,57, 5,972,60, 6,136,53 and 5,955,26, among others.

Allele Specific Oligonucleotide (ASO)-Dot Blot Analysis

Another method for detecting polymorphisms on specific genes is that following allele specific oligonucleotide (ASO)-dot blot analysis (Conner, B. J. et al., Proc. Natl. Acad. Sci., U.S.A., 80, 278-282 (1983)). This method can be performed by subjecting a DNA fragment hybridizable with oligonucleotide probes specific to an allele of a PCR amplified gene fragment, using forward primers and reverse primers designed so as to sandwich a target polymorphism, to dot blot analysis. In this way, the polymorphism on such DNA fragments can be detected.

Polypeptide Analysis

When polymorphism involves changes of amino acids, analysis of polymorphism is possible using an expression product of the gene to be analyzed. In this case, a partial protein or partial peptide may be used as the sample for analysis so long as it contains an amino acid corresponding to the polymorphism site. A method of directly assaying the amino acids at the polymorphism site or an immunological method may be used in this case. Known amino acid sequence analysis method such as Edman method may be used in the former, while a method using a monoclonal or polyclonal antibody having bonding activity specific to the expression product of the gene, for example an enzyme-linked immunosorbent assay method (ELISA method), radio-immunoassay, immunoprecipitation method or immunodiffusion method, may be used in the latter.

Information on polymorphism obtained as described above may be statistically totaled and used for diagnosis of disease, detection and discrimination of the relative risk for the morbidity and selection of remedies.

Expression Analysis

A number of methods are available for determining the expression level of a polymorphic BIM nucleic acid molecule, e.g., a polymorphic BIM mRNA, or polymorphic BIM polypeptide in a particular sample. Diagnosis may be performed by a number of methods to determine the absence or presence or altered amounts of normal or abnormal BIM mRNA in a patient sample. For example, detection may utilize staining of cells or histological sections with labeled antibodies, performed in accordance with conventional methods. Cells are permeabilized to stain cytoplasmic molecules. The antibodies of interest are added to the cell sample, and incubated for a period of time sufficient to allow binding to the epitope, usually at least about 10 minutes. The antibody may be labeled ith radioisotopes, enzymes, fluorescers, chemiluminescers, or other labels for direct detection. Alternatively, a second stage antibody or reagent is used to amplify the signal. Such reagents are well known in the art. For example, the primary antibody may be conjugated to biotin, with horseradish peroxidase-conjugated avidin added as a second stage reagent. Alternatively, the secondary antibody conjugated to a fluorescent compound, e.g. fluorescein, rhodamine, Texas red, etc. Final detection uses a substrate that undergoes a color change in the presence of the peroxidase. The absence or presence of antibody binding may be determined by various methods, including flow cytometry of dissociated cells, microscopy, radiography, scintillation counting, etc. The presence and/or the level of a polymorphic BIM polypeptide may also be detected and/or quantitated in any way known to one of ordinary skill.

In addition, a test can include measurements of the expression of BIM mRNA. Biochemical studies may be performed to determine whether a sequence polymorphism in a BIM coding region or control regions is associated with disease. Disease associated polymorphisms may include deletion or truncation of the gene, mutations that alter expression level, that affect the activity of the protein, etc.

Changes in the promoter or enhancer sequence that may affect expression levels of BIM can be compared to expression levels of the normal allele by various methods known in the art. Methods for determining promoter or enhancer strength include quantitation of the expressed natural protein; insertion of the variant control element into a vector with a reporter gene such as β-galactosidase, luciferase, chloramphenicol acetyltransferase, etc. that provides for convenient quantitation; and the like.

Screening for mutations in a polymorphic BIM polypeptide may be based on the functional or antigenic characteristics of the protein. Protein truncation assays are useful in detecting deletions that may affect the biological activity of the protein. Various immunoassays designed to detect polymorphisms in polymorphic BIM polypeptides may be used in screening. Where many diverse genetic mutations lead to a particular disease phenotype, functional protein assays have proven to be effective screening tools. The activity of the encoded a polymorphic BIM polypeptide may be determined by comparison with a reference BIM polypeptide lacking a specific polymorphism.

Diagnostic methods in which the level of BIM gene expression is of interest will typically involve comparison of the BIM nucleic acid abundance of a sample of interest with that of a control value to determine any relative differences, where the difference may be measured qualitatively and/or quantitatively, which differences are then related to the presence or absence of an abnormal BIM gene expression pattern. A variety of different methods for determine the nucleic acid abundance in a sample are known to those of skill in the art, where particular methods of interest include those described in: Pietu et al., Genome Res. (June 1996) 8: 492-503; Zhao et al., Gene (Apr. 24, 1995) 156: 207-213; Soares, Curr. Opin. Biotechnol. (October 1997) 8: 542-546; Raval, J. Pharmacol Toxicol Methods (November 1994) 32: 125-127; Chalifour et al., Anal. Biochem (Feb. 1, 1994) 216: 299-304; Stolz & Tuan, Mol. Biotechnol. (December 1960 6: 225-230; Hong et al., Bioscience Reports (1982) 2: 907; and McGraw, Anal. Biochem. (1984) 143: 298. Also of interest are the methods disclosed in WO 97/27317, the disclosure of which is herein incorporated by reference.

### BIM (BCL2L11)

BIM is also known as BAM; BIM; BOD; BimL; BimEL; BIM-beta6; BIM-beta7; BIM-alpha6; BCL2L11.

GenBank Accession numbers and UCSC Gene IDs of the BIM gene and polypeptide are as follow:

GeneBank Accession Numbers:

| Gene (transcript) | Protein |
|---|---|
| NM_207002 | NP_996885 |
| NM_138621 | NP_619527 |
| NM_006538 | NM_006538 |

UCSC Gene IDs:

| Gene (transcript) | Protein |
|---|---|
| uc002tgw.1 | No protein |
| uc002tgx.1 | No protein |
| uc010fkd.1 | No protein |
| uc002tgy.1 | No protein |
| uc002tgz.1 | O43521 |
| uc010fke.1 | No protein |
| uc002tha.1 | O43521 |
| uc002thb.1 | No protein |
| uc002thc.1 | No protein |
| uc002thd.1 | O43521 |

The following transcript descriptions correspond to the Gene Bank accession numbers and UCSC Gene IDs listed above.

NCBI GeneBank (RefSeq)

NM_{—}207002.2

Homo sapiens BCL2-like 11 (apoptosis facilitator) (BCL2L11), transcript variant 9, mRNA.

NM_{—}138621.3

Homo sapiens BCL2-like 11 (apoptosis facilitator) (BCL2L11), transcript variant 1, mRNA.

NM_{—}006538.3

Homo sapiens BCL2-like 11 (apoptosis facilitator) (BCL2L11), transcript variant 6, mRNA.

UCSC Genes

bim-alpha1 (uc002tgw.1)

Bim-alpha1, complete cds.

RefSeq (NM_{—}006538):

bim-alpha1 (uc002tgx.1)

Bim-alpha1, complete cds.

RefSeq (NM_{—}006538):

bim-alpha1 (uc010fkd.1)

Bim-alpha1, complete cds.

RefSeq (NM_{—}006538):

bim-alpha1 (uc002tgy.1)

Bim-alpha1, complete cds.

RefSeq (NM_{—}006538):

bim-alpha1 (uc002tgz.1)

BCL2-like 11 transcript variant 9.

RefSeq (NM_{—}006538)

bim-alpha1 (uc010fke.1)

Bim-alpha1, complete cds.

RefSeq (NM_{—}006538):

BCL2L11 (uc002tha.1)

BCL2-like 11 isoform 9

RefSeq (NM_{—}138621):

bim-alpha1 (uc002thb.1)

Bim-alpha1, complete cds.

RefSeq (NM_{—}006538):

bim-alpha1 (uc002thc.1)

Bim-alpha1, complete cds.

RefSeq (NM_{—}006538):

BCL2L11 (uc002thd.1)

BCL2-like 11 isoform 9

RefSeq (NM_{—}006538):

Literature UCSC Genome Browser Database/Data

Rhead B, Karolchik D, Kuhn R M, Hinrichs A S, Zweig A S, Fujita P, Diekhans M, Smith K E, Rosenbloom K R, Raney B J, Pohl A, Pheasant M, Meyer L, Hsu F, Hillman-Jackson J, Harte R A, Giardine B, Dreszer T, Clawson H, Barber G P, Haussler D, Kent W J. The UCSC Genome Browser database: update 2010. Nucleic Acids Res. 2010 January; 38(Database issue):D613-9. Epub 2009 Nov. 11.

The protein encoded by this gene belongs to the BCL-2 protein family. BCL-2 family members form hetero- or homodimers and act as anti- or pro-apoptotic regulators that are involved in a wide variety of cellular activities. The protein encoded by this gene contains a Bcl-2 homology domain 3 (BH3). It has been shown to interact with other members of the BCL-2 protein family, including BCL2, BCL2L1/BCL-X(L), and MCL1, and to act as an apoptotic activator. The expression of this gene can be induced by nerve growth factor (NGF), as well as by the forkhead transcription factor FKHR-L1, which suggests a role of this gene in neuronal and lymphocyte apoptosis. Transgenic studies of the mouse counterpart suggested that this gene functions as an essential initiator of apoptosis in thymocyte-negative selection. Several alternatively spliced transcript variants of this gene have been identified. [provided by RefSeq]

### Prediction of Resistance to Tyrosine Kinase Inhibitor Treatment in Chronic Myelogenous Leukaemia (CML)

Our discovery may have clinical utility in the following circumstances.

Patients with BIM deletions may benefit from more frequent monitoring of their condition. This may allow earlier use of alternative and/or more aggressive therapies, e.g. bone marrow transplantation, and can potentially lead to a better clinical outcome.

BIM deletions may serve as a therapeutic guide. For example, patients with the deletion may represent a subgroup of TKI-resistant patients who may be particularly sensitive to a class of drugs known as BH3-mimetics (refs A8-A12). Such drugs, like ABT-263 (currently in early phase clinical trials), selectively target pro-survival members of the BCL2 family of proteins, and which would normally be opposed by the pro-death family of BIM proteins.

CML is a cancer of haematopoietic stem cells, and is caused by the presence of the oncogenic fusion gene, termed BCR-AB L, that is thought to be causative for the disease. BCR-ABL encodes for a constitutively active tyrosine kinase that mediates the increased survival and proliferation of CML cells when compared to their normal counterparts.

Effective treatment for CML exists in the form of a class of drugs which inhibit the kinase activity of BCR-AB L, which are commonly called tyrosine kinase inhibitors (TKI). However, over time, a proportion of patients develop clinical resistance to these drugs, an event that is often associated with transformation to blast crisis (BC) CM L, and shorter survival.

In about 60% of patients, TKI-resistance is associated with reactivation of BCR-ABL through mutations in the BCR-ABL gene that render TKIs less able to bind to BCR-AB L, or overexpression of the ‘wild type’ BCR-ABL gene or protein. In both cases, BCR-ABL kinase activity is restored in the presence of the TKI, hence the term BCR-ABL-dependent TKI-resistance. In the remaining 40% of cases, TKI-resistance occurs in the absence of BCR-ABL reactivation, and is termed BCR-ABL-independent TKI-resistance.

To date, the causes of BCR-ABL-independent TKI-resistance are unknown, and an increased understanding of the mechanisms that mediate this form of resistance will be important to determine strategies to overcome TKI-resistance. Because TKI-resistance might be mediated by either acquired or inherited genetic differences between patients who develop TKI-resistance compared to those who do not, we reasoned that identification of such differences would be a promising approach to uncover mechanisms of TKI-resistance.

Accordingly, we applied a novel technology termed genomic paired-end ditag or DNA-PE T, coupled with high-throughput sequencing, to interrogate the genome of primary CML cells from patients with or without drug-resistance, and with and without BCR-ABL kinase domain mutations.

In this disclosure, we describe the following novel discoveries made by our team.

Through the use of DNA-PE T, we have uncovered a previously unknown deletion in the BIM gene that is associated with the development of BCR-ABL-independent TKI-resistance. Using a clinically annotated set of CML patient samples, we find that patients with this deletion are four to five times more likely to have BCR-ABL-independent TKI-resistance than those without the deletion (p=0.04, two-sided Fisher's exact test).

Secondly, we have found that the presence of the BIM deletion in patient-derived CML cell lines is strongly associated with BCR-ABL-independent TKI-resistance. Using a panel of 7 patient-derived CML cell lines, we have found one cell line with the deletion. Importantly, only the BIM-deletion containing cell line exhibited BCR-ABL-independent TKI-resistance. These results suggest a direct mechanistic role for BIM deletions in BCR-ABL-independent TKI-resistance. Since it is known that upregulation of BIM protein is required for TKI-mediated CML cell death, we hypothesize that BIM deletions impair the expression of proapoptotic isoforms of BIM.

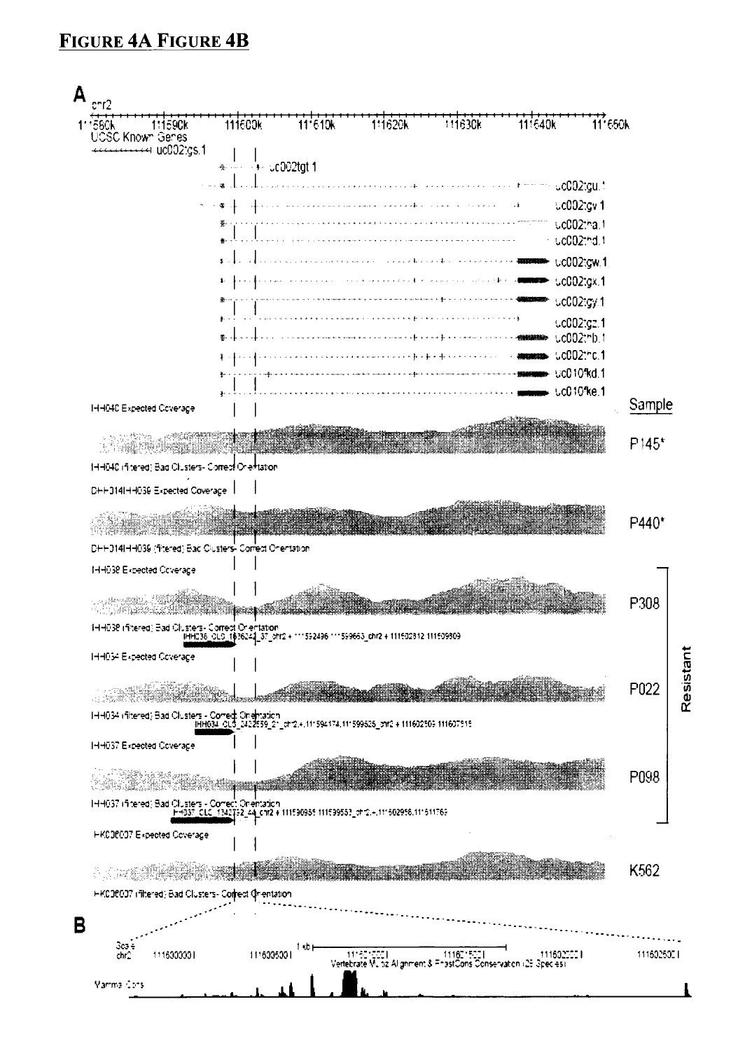

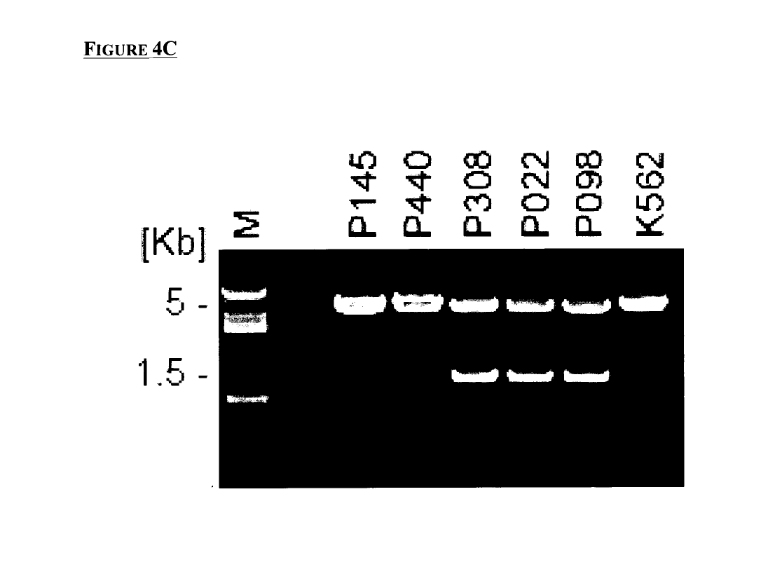

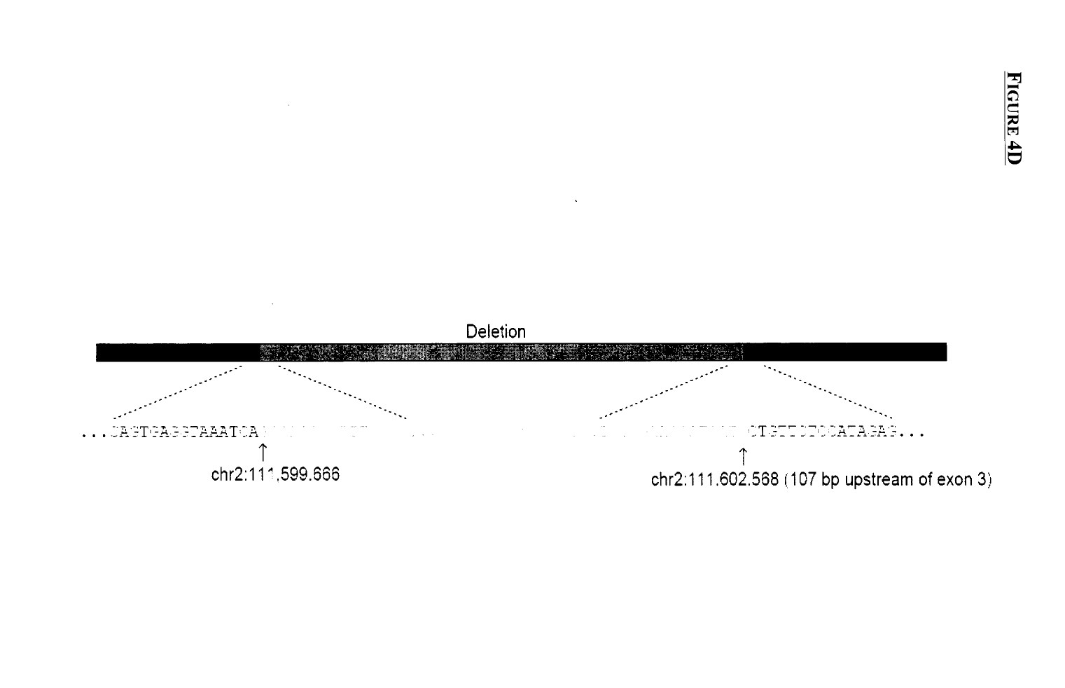

Further analysis of the BIM deletion (using the PCR assay as described in Example 2, with the primers described by Sequences No. 3 and No. 4 as shown in FIG. 4C, SNP analysis and HapMap data) has revealed that the deletion is actually a normal polymorphism that it has a frequency of approximately 10% in individuals of East-Asian descent. The deletion polymorphism is likely to be non-existent in Caucasians, based on the screening of 60 Caucasian HapMap samples and 446 German blood donors, or African (Yoruba) populations based on the screening of 60 Yoruban HapMap samples (Table E6). Consistent with the ethnic segregation of the BIM deletion, the cell line with the deletion described in b. was found to have been derived from a Japanese individual with CML (ref A31).

TABLE E6
Genotype and allele frequency of the deletion
polymorphismus in HapMap and German individuals.

| | | | | Allele |
|---|---|---|---|---|
| | Genotype | | | frequency |
| | wt/wt | wt/del | del/del | del |
| European, HapMap (n = 60) | 60 | 0 | 0 | 0 |
| East Asian, HapMap (n = 74) | 61 | 12 | 1 | 0.095 |
| African, HapMap (n = 60) | 60 | 0 | 0 | 0 |
| German (n = 446) | 446 | 0 | 0 | 0 |

This finding has several implications and potential clinical uses, as described in the following sections.

The invention may be used as follow:

It is currently not possible to predict at presentation which CML patients will develop TKI-resistance. By identifying a genetic factor that is associated with an increased risk of developing TKI-resistance, it may now be possible, in East-Asian populations, to identify such individuals. Patients with BIM deletions would then be followed more closely by their physicians, and may be advised to have more frequent monitoring of their disease status than is currently recommended. These would include more frequent blood and bone marrow tests.

In addition, the presence of the BIM deletion may predict for sensitivity to specific alternative therapeutic strategies. These include increasing the dose of imatinib beyond the standard dose of 400 mg/day to 600 or 800 mg/day. Alternatively, such patients may potentially be treated with a novel class of drugs that inhibit the pro-survival effect of the BCL2 group of proteins (which the BIM protein normally opposes). This latter possibility is currently being explored in our laboratory.

Importantly, because TKIs and other targeted therapies are very costly, physicians managing patients who are found to have BIM deletions, may use this finding as a rationale for justifying the increased cost associated with the use of higher drug doses, or changing therapies, to their patients and/or third party payors, or avoiding these strategies in the absence of a deletion.

### Other Diseases

The presence of BIM deletions in patients with other cancers may also be used as a predictor for the development of drug-resistance, as well as a guide for alternative therapies.

This would be analogous to the situation described for CM L, and may encompass other haematologic malignancies (chronic lymphocytic leukaemia, acute lymphoblastic leukaemia, acute myeloid leukaemia, and multiple myeloma), myeloproliferative disorders (including polycythaemia vera, essential thrombocythaemia, and primary myelofibrosis), as well as the most common solid tumours (non-small cell and small cell lung cancer, breast cancer, colorectal cancer, ovarian cancer, melanoma, and neuroblastoma), and gastrointestinal stromal tumours (GIST).

Thus, the BIM polymorphism disclosed here may also be used to detect resistance to kinase inhibitor treatment in other diseases, such as EGFR-driven non-small cell lung cancers (NSCLC) and gastrointestinal stromal tumours (GIST, Gordon and Fisher, 2010).

Accordingly, patients with such cancers and tumours and who harbour the BIM polymorphism disclosed here are more likely to be resistant to therapies targeting their respective oncogenic kinases.

Furthermore, reference is made to Will et al. Apoptosis induced by JAK2 inhibition is mediated by Bim and enhanced by the BH3 mimetic ABT-737 in JAK2 mutant human erythroid cells. Blood 8 Apr. 2010 Vol 115, number 14. This document describes myeloproliferative disorders, which are characterized by JAK2 activating mutations. This article provides evidence that JAK inhibitors also require BIM expression for sensitivity.

Accordingly, the BIM polymorphism disclosed here may further be used to detect resistance to kinase inhibitor treatment in myeloproliferative disorders, such as polycythaemia vera, essential thrombocythaemia, and primary myelofibrosis. Patients with myeloproliferative disorders such as those harbouring the BIM polymorphism disclosed here are more likely to be resistant to therapies targeting their respective oncogenic-kinases.

We disclose a polymorphic variant of a BIM (BCL2L11) gene which is associated with resistance to treatment with a kinase inhibitor in an individual suffering from an EGFR-driven non-small cell lung cancer (NSCLC), a c-KIT/PDGFR-driven gastrointestinal stromal tumour (GIST) or a JAK2 driven myeloproliferative disorder, in an individual comprising such a polymorphism.

Examples of kinase inhibitors used to treat EGFR-driven non-small cell lung cancers (NSCLC) include gefitinib and erlotinib, while an example of a kinase inhibitor used to treat gastrointestinal stromal tumours (GIST) is imatinib. Myeloproliferative disorders may be treated with kinase inhibitors against their causative kinases, e.g. inhibitors against JAK2.

The BIM polymorphism may comprise a polymorphic variant of a BIM (BCL2L11) gene which comprises, in 5′ to 3′ order, the nucleotide sequence set out in SEQ ID NO: 5 followed immediately by the nucleotide sequence set out in SEQ ID NO: 7. The polymorphic variant BIM (BCL2L11) may be characterised by lacking the nucleotide sequence set out in SEQ ID NO: 6.

We disclose a method of predicting whether an individual suffering from a non-small cell lung cancer (NSCLC) is likely to develop resistance to treatment with a kinase inhibitor, the method comprising determining whether the individual has a BIM (BCL2L11) polymorphism as described. The resistance may be independent of EGFR reactivation. The non-small cell lung cancer (NSCLC) may comprise a non-small cell lung cancer (NSCLC) associated or caused by EGFR activation such as an EGFR driven non-small cell lung cancer (NSCLC).

We further disclose a method of predicting whether an individual suffering from a gastrointestinal stromal tumour (GIST) is likely to develop resistance to treatment with a kinase inhibitor, the method comprising determining whether the individual has a BIM (BCL2L11) polymorphism as described. The resistance may be independent of c-KIT/PDGFR reactivation. The gastrointestinal stromal tumour (GIST) may comprise a gastrointestinal stromal tumour (GIST) associated or caused by c-KIT/PDGFR activation such as a c-KIT/PDGFR driven gastrointestinal stromal tumour (GIST).

We also disclose a method of predicting whether an individual suffering from a or a myeloproliferative disorder is likely to develop resistance to treatment with a kinase inhibitor, the method comprising determining whether the individual has a BIM (BCL2L11) polymorphism as described. The resistance may be independent of JAK2 reactivation. The myeloproliferative disorder may comprise a myeloproliferative disorder associated or caused by JAK2 activation such as a JAK2 driven myeloproliferative disorder.

We disclose a method comprising detecting the presence of a nucleic acid amplification product comprising a sequence set out in SEQ ID NO: 1, for example by use of a primer set comprising a nucleotide sequence as set out in SEQ ID NO: 3 and a nucleotide sequence as set out in SEQ ID NO: 4, in which if the individual is determined to have the BIM (BCL2L11) polymorphism, then the individual is likely to develop resistance to treatment of a non-small cell lung cancer (NSCLC) with a kinase inhibitor.

We further disclose such a method, in which if the individual is determined to have the BIM (BCL2L11) polymorphism, then the individual is likely to develop resistance to treatment of a gastrointestinal stromal tumour (GIST) with a kinase inhibitor.

We also disclose such a method, in which if the individual is determined to have the BIM (BCL2L11) polymorphism, then the individual is likely to develop resistance to treatment of a myeloproliferative disease with a kinase inhibitor.

Conversely, if such an individual is determined not to have the BIM (BCL2L11) polymorphism (for example if the presence of a nucleic acid amplification product comprising a sequence set out in SEQ ID NO: 2 is detected), then the individual is less likely to develop resistance to treatment of a non-small cell lung cancer (NSCLC) with a kinase inhibitor.

Similarly, in such a method, in which if the individual is determined not to have the BIM (BCL2L11) polymorphism, then the individual is less likely to develop resistance to treatment of a gastrointestinal stromal tumour (GIST) with a kinase inhibitor.

Also, in such a method, in which if the individual is determined not to have the BIM (BCL2L11) polymorphism, then the individual is less likely to develop resistance to treatment of a meyloproliferative disease with a kinase inhibitor.

We describe a method of determining the likelihood of success of a particular therapy on an individual with non-small cell lung cancer (NSCLC) or gastrointestinal stromal tumour (GIST) or myeloproliferative disease, or any combination of the above, the method comprising comparing the therapy with the therapy determined by a method set out above.

We further describe a method of diagnosis of kinase inhibitor resistant non-small cell lung cancer (NSCLC) in an individual, the method comprising detecting the presence of a BIM (BCL2L11) polymorphism, as described above, in an individual. We also describe a method of diagnosis of kinase inhibitor resistant gastrointestinal stromal tumour (GIST) in an individual, the method comprising detecting the presence of a BIM (BCL2L11) polymorphism, as described above, in an individual. We describe a method of diagnosis of kinase inhibitor resistant myeloproliferative disease in an individual, the method comprising detecting the presence of a BIM (BCL2L11) polymorphism, as described above, in an individual.

We provide for method of treatment of a patient suffering from non-small cell lung cancer (NSCLC) and/or gastrointestinal stromal tumour (GIST) and/or a myeloproliferative disease, the method comprising determining whether the cancer is kinase-resistant cancer by a method described above and treating the patient.

### EXAMPLES

### Example 1

### Assay for Screening for the Presence of the Deletion in Bim-Alpha1

Standard Protocols/Kits for DNA extraction

Qiagen Blood & Cell Culture DNA Midi Kit (cat. No. 13343) for the DNA extraction from blood (see QIAGEN_Genomic_DNA_Handbook.pdf for details).

or

MasterAmp™ Buccal Swab DNA Extraction Kit for DNA extraction from buccal swabs.

It is also possible to use the Qiagen Allprep DNA/RNA mini kit (cat. No 80204). Qiagen Inc, Valencia, Calif., United States of America.

### Example 2

### PCR Assay Using Genomic DNA for Bim Deletion Detection

| | | |
|---|---|---|
| Primers (100uM) | 0.2 | μl each |
| Genomic DNA | 1 | μl (50 ng) |
| dNTP (10 mM) | 2.5 | μl |
| Jumpstart Tag | 2.5 | μl (Sigma; Cat. D1313) |
| 10 × Buffer | 5 | μl |
| Add H₂O to | 50 | μl |

PCR program

Step 1: 96° C. for 30sec

Step 2: 94° C. for 15sec

Step 3: 64° C. for 30sec

Step 4: 68° C. for 5 min

Step 5: Repeat steps 2-4×11

Step 6: 94° C. for 15sec

Step 7: 60° C. for 30sec

Step 8: 68° C. for 5 min

Step 9: Repeat steps 6-8×17

Step 10: 68° C. 20 mins

16° C. forever

Primers
Bim_del_F
AATACCACAGAGGCCCACAG
(+chr2: 111,599,051 . . . 111,599,070)
Bim_del_R
GCCTGAAGGTGCTGAGAAAG
(−chr2: 111,603,257 . . . 111,603,276)

PCR products are run on a 1% agarose gel with ethidium bromide and products of the sizes

4,226 bp without deletion

1,323 bp with deletion

are visualised on a UV screen.

### Example 3

### Materials and Methods: Patients and Samples

Clinical samples were obtained from patients seen at the Singapore General Hospital and University of Malaya according to IRB-approved protocols.

Mononuclear cells were isolated by Ficoll centrifugation, and DNA/RNA extracted using AllPrep DNA/RNA Mini Kit (Qiagen) according to the manufacturer's instructions.

### Example 4

### Materials and Methods: DNA-PET Analysis and Validation

Genomic DNA was hydrosheared to 5, 7, and 9 Kb fragments, used for construction of sequencing libraries, and sequenced using a massively parallel SOLiD sequencer (see Table E1 and description below).

TABLE E1
Statistics of massively parallel PET sequencing on the SOLiD platform

| | Sam- | | Mappable | | | cPET² span | | Cover- | dPET⁴ | dPET⁴ |
|---|---|---|---|---|---|---|---|---|---|---|
| | ple | Tags | Tags | PET | PET (NR)¹ | range [bp] | cPET (NR)¹ | age³ | (NR)¹ | cluster⁵ |
| DHH014IHH039 | P440 | 1,100,736,719 | 654,761,718 | 237,888,286 | 60,483,853 | 6,430-10,460 | 54,566,588 | 156.7 | 5,917,265 | 1,255 |
| IHH040 | P145 | 406,158,202 | 187,588,800 | 57,282,295 | 30,574,510 | 7,339-10,440 | 27,920,763 | 86.5 | 2,653,747 | 782 |
| IHH038 | P308 | 983,240,880 | 486,558,654 | 137,899,606 | 39,548,846 | 5,930-8,090 | 31,886,502 | 77.8 | 7,662,344 | 1,211 |
| IHH037 | P098 | 856,013,096 | 477,870,346 | 132,002,088 | 53,906,020 | 7,655-10,272 | 45,363,132 | 143.3 | 8,542,888 | 1,444 |
| IHH034 | P022 | 1,536,669,332 | 854,022,398 | 256,381,192 | 50,259,818 | 4,410-6,429 | 38,068,014 | 69.7 | 12,191,804 | 1,555 |
| IHK006007E | K562 | 376,077,182 | 223,850,946 | 133,675,193 | 44,065,447 | 6,846-10,248 | 40,261,305 | 118.4 | 3,804,142 | 1,153 |

¹non redundant
²concordant PET
³physical coverage
⁴discordant PET
⁵filtered clusters based on in silico cluster size probability

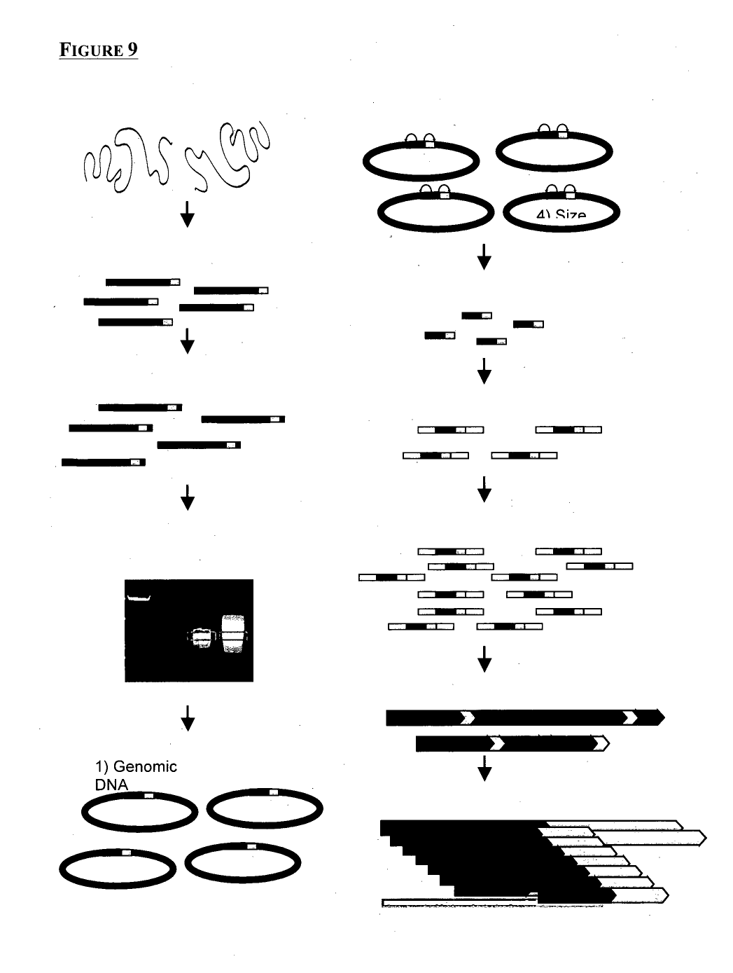

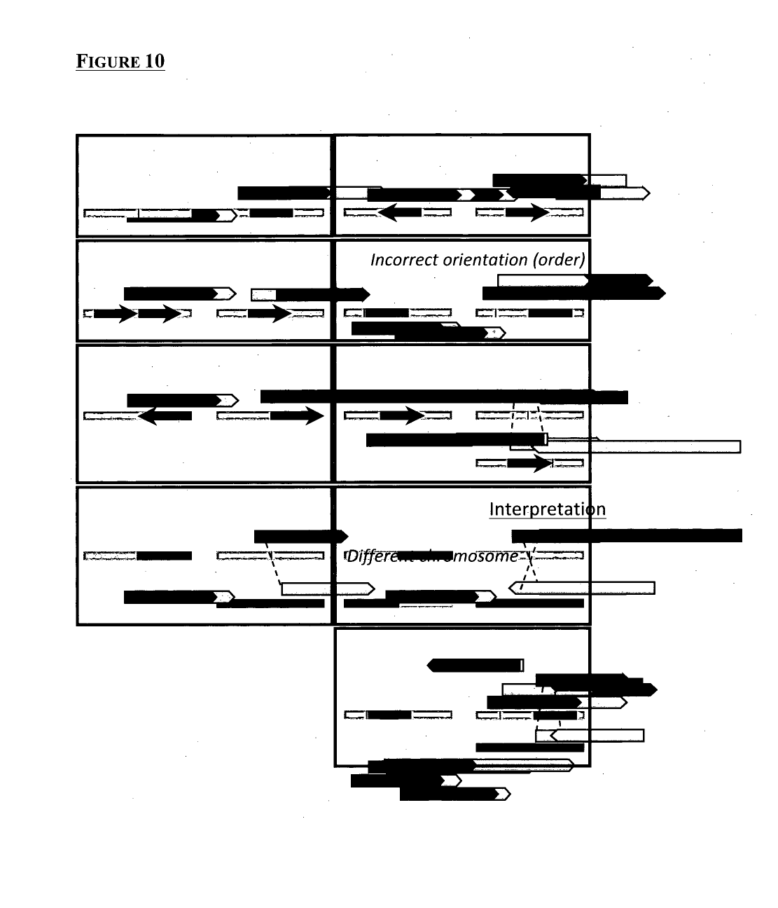

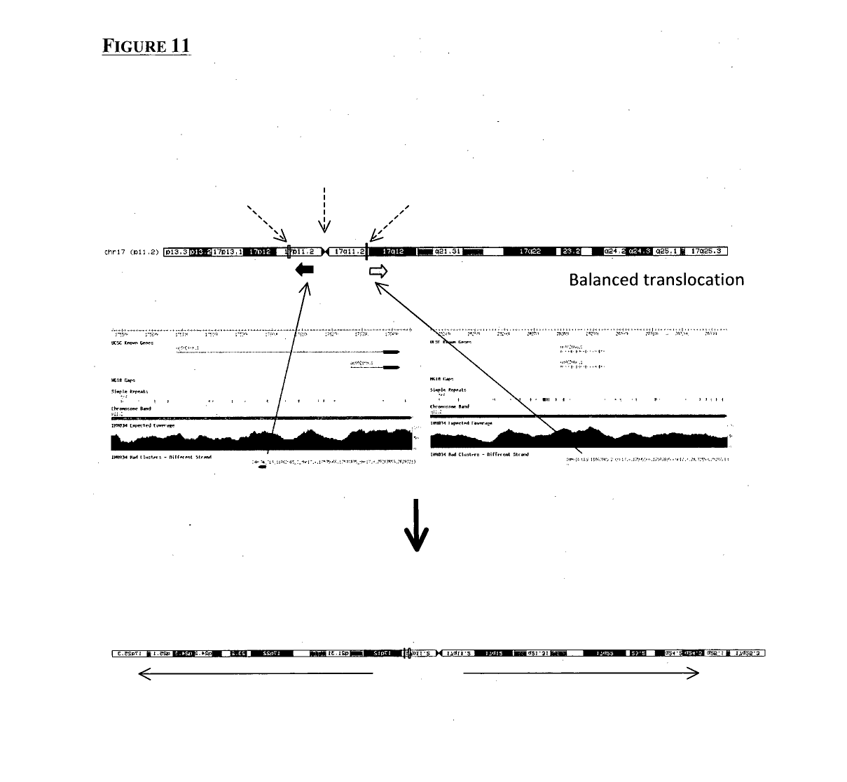

Paired-end (Applied Biosystems terminology: mate-paired) libraries were constructed as shown in FIG. 9. In brief, 2×25 bp ditag constructs, which corresponded to the ends of the 5-9 Kb DNA fragments, were generated using EcoP15I. High throughput sequencing of the 2×25 bp libraries was performed on SOLiD sequencers according to the manufacturer's recommendations (Applied Biosystems). Sequence tags were mapped to the human reference sequence (NCBI Build 36, hg18) and paired using SOLiD System Analysis Pipeline Tool, Corona Lite (Applied Biosystems), allowing 2 color code mismatches per tag. Paired-end tags (PETs) which were within the library insert distribution were categorized as concordant PETs (cPETs). The PETs which were rejected by cPET criteria were classified as discordant PETs (dPETs). These were further split into five distinct categories; (i) two tags mapped on different chromosome, (ii) two tags mapped on the same chromosome, but different strand, (iii) two tags mapped on the same chromosome, but wrong ordering (5′ downstream of 3′), (iv) two tags mapped on the same chromosome, same strand, correct ordering, but larger span distance than 1.1× the maximum library size, (v) two tags mapped on same chromosome, same strand, correct ordering, but smaller span distance than the minimum library size. Category (v) has been excluded from further analysis. dPETs of which the sequence tags mapped on both sides to the same genomic regions within 10 Kb were clustered together. The 10 Kb search windows to the left and right of the first tags were extended to 10 Kb according to the outermost tag coordinates. The regions to which the left (5′) and right (3′) tags mapped were described as anchor regions. Clusters of the size 3 and higher were kept for the identification of SVs. Single dPET clusters could identify SVs with one rearrangement point such as deletions if the 5′ mapping anchor region was far apart from the 3′ mapping anchor region, tandem duplications if the mapping order was 3′ to 5′ instead of the normal 5′ to 3′, unpaired inversion if the mapping orientation was reversed (on different strand), and isolated inter-chromosomal translocation if the 5′ and 3′ anchors mapped to different chromosomes. Two closely positioned dPET clusters could be used to deduce the SVs with two rearrangement points such as inversions, insertions, and balanced translocations as described in FIG. 10.

Comparison of clusters across different genomes was performed based on an overlap of the 5′ and 3′ anchor regions extended by 10 Kb on both sides. If the 5′ anchor region of a cluster of a second library was overlapping with the 5′ extended anchor region of a cluster of the first library and the same was true for the 3′ anchor regions, the two clusters were grouped together and the 10 Kb extension of the anchor regions were adjusted according to the outermost start and end anchor coordinates. Gene annotations were based on RefSeq Genes downloaded from UCSC (http://genome.ucsc.edu/; Rhead et al. 2010) on May 14, 2009 using library specific breakpoints. Identified SVs were filtered based on SVs of 32 normal individuals and DNA-PET quality criteria as described elsewhere in this document.

Some loci showed an accumulation of dPET clusters. At these loci it might be misleading to assign a particular SV to a dPET cluster (e.g. if the breakpoints of a tandem duplication are surrounded by deletions and/or translocations, the rearrangement might not be interpreted as a tandem duplication). Therefore, a breakpoint based interconnection network was established to separate breakpoints in complex regions from isolated and less complex SVs. To determine the neighbourhood of a breakpoint, the start and end points of each dPET cluster anchor region were extended by the maximum insert size of the respective genomic library as search windows. If windows of neighbouring clusters overlapped with each other, dPET clusters were grouped together into a supercluster. The procedure allowed an indirect connection of cluster A via B to C. The number of dPET clusters that could be joined together into a supercluster was represented by the supercluster size. In cases where >3 dPET clusters were interconnected, the breakpoint pairs were classified as ‘complex’ (intra- and inter-chromosomal).

Fluorescence in situ hybridization (FISH) was performed as follows. Nuclei were harvested by treating cells with 0.75M KCl for 20 min at 37° C. Then, after few fixations, nuclei were dropped on slides for FISH. EVI1 probe (clone RP11-137H17) was labelled green and insertion of chr8: 127,950,637 to 130,664,919 (clones RP11-828L6 and RP11-159N7, respectively) was labelled in red and yellow, respectively. For fosmid probe preparation, DNA was labelled by nick translation in the presence of biotin-16-dUTP using Nick translation system (Invitrogen). In presence of 1 μg/μl of Cot1 DNA, DNA cosmid clone was resuspended at a concentration of 5 ng/μl in hybridization buffer (2SSC, 10% dextran sulfate, 1×PBS, 50% formamide). Prior to hybridization, nuclei slides were treated with 0.01% pepsin at 37° C. for 5 min followed by 1×PBS rinse, 1% formaldehyde 10 min treatment, 1×PBS rinse (5 min) and dehydratation through ethanol series (70%, 80%, and 100%). Denaturated probe was applied to these pretreated slides and codenaturated at 75° C. for 5 min and hybridized at 37° C. overnight. Two posthybridization washes were performed at 45° C. in 2SSC/50% formamide for 7 min each followed by 2 washes in 2SSC at 45° C. for 7 min each. After blocking, the slides were revealed with avidin-conjugated fluorescein isothiocyanate (FITC) (Vector Laboratories). After washing, slides were mounted with vectashield and observed under epifluorescence microscope. Image analysis was done using Metasystem software.

For validation of predicted SVs, PCR primers flanking the predicted break points were used to amplify rearranged regions for subsequent Sanger sequencing.

For real-time PCR assays, total cellular RNAs were extracted using the RNeasy Mini Kit (Qiagen) according to the manufacturer's instruction. RNA was reverse transcribed using Superscript III First-Strand Synthesis System (Invitrogen), and quantitatively assessed using the iQ5 Multicolor Real-Time Detection System (Bio-Rad) with a total reaction volume of 25 ul. Primers were annealed at 59° C. for 20 seconds and the amplicon was extended at 72° C. for 30 seconds. The total number of cycles quantified was 40. Transcript levels of β-actin or exon 2A of BIM were used to normalize between samples. Primers used are as the following: BIM exon 2A (Forward: ATGGCAAAGCAACCTTCTGATG; Reverse: GGCTCTGTCTGTAGGGAGGT), BIM exon 3 (Forward: CAATGGTAGTCATCC-TAGAGG; Reverse: GACAAAATGCTCAAGGAAGAGG), BIM exon 4 (Forward: TTCCATGAGGCAGGCTGAAC; Reverse: CCTCCTTGCATAGTAAGCGTT), β-actin (Forward: GGACTTCGAGCAAGAGATGG; Reverse: AGCACTGTGTTGGCGTAC-AG) and EVI1 (Forward: ACCCACTCCTTTCTTTATGGACC; Reverse: TGATC-AGGCAGTTGGAATTGTG).

### Example 5

### Materials and Methods: Cell Lines and Tissue Culture

CML lines were obtained from ATCC (MEG-01 and KU812), JCRB (NCO2) and DSMZ (KCL22, K562, KYO1, JK1, BV173 and NALM1). Cells were grown in RPMI 1640 medium supplemented with Penicillin, Streptomycin, Glutamine and 10% fetal bovine serum, incubated in humidified incubator at 37° with 5% CO₂.

### Example 6

### Results: CML Patient Samples

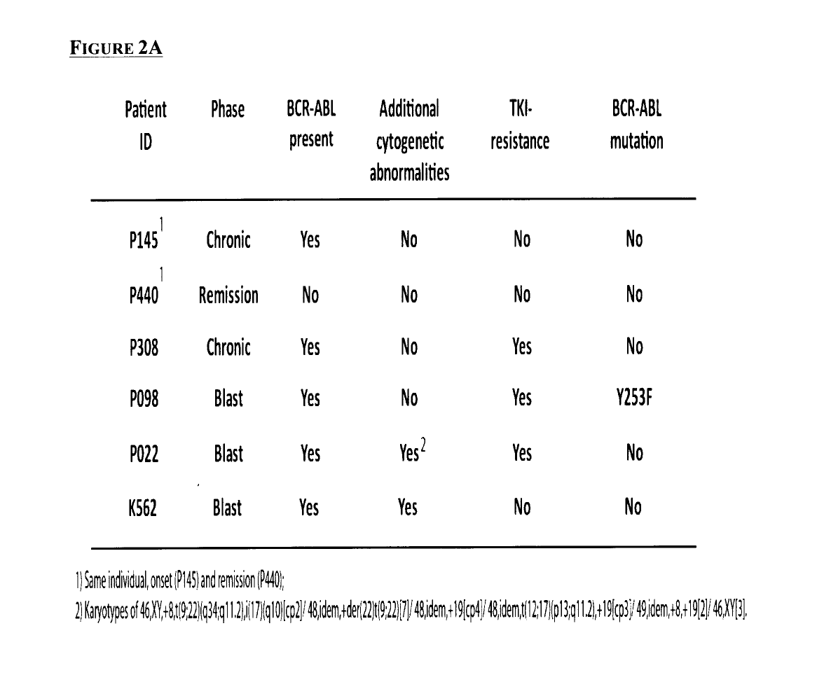

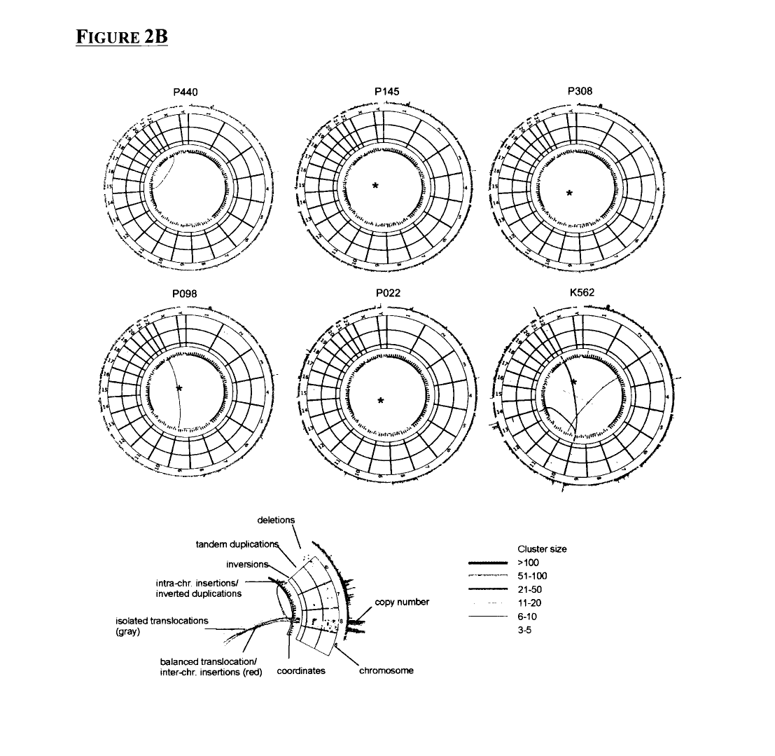

We selected four Philadelphia (Ph) chromosome-positive CML patient samples and one CML cell line for DNA-PET analysis (FIG. 2A).

These samples represent patients exhibiting clinical sensitivity or resistance to tyrosine kinase inhibitors, and include two patients each in chronic and myeloid blast phase. One of the chronic phase patients and both blast phase patients were resistant to tyrosine kinase inhibitors, while one of the blast phase samples displayed additional karyotypic abnormalities besides the Ph chromosome.

We also included the K562 CML cell line and a remission sample from the treatment-sensitive patient as positive and negative controls respectively for the Ph chromosome.

### Example 7

### Results: DNA-PET Analysis of CML Genomes

We generated 72.1 Gb of mappable DNA sequence derived from >278 million non-redundant PETs and achieved, on average, 109-fold physical (fragment) coverage of each genome (Table E1 above). 85.4% of the PETs mapped concordantly to the reference genome, while 14.6% of the PETs did not. The latter were classified as discordant PETs (dPETs). The clustering of multiple dPETs connecting the same two genomic regions allowed us to identify 3,408 different structural variations (SV), as well as define the type of SV for each dPET (see FIG. 10, and Tables E2 and E3 below).

TABLE E2
SVs predicted by DNA-PET in 5 patient samples and K562

| | P440 | P145 | P308 | P098 | P022 | K562 |
|---|---|---|---|---|---|---|
| Deletion | 434 | 413 | 510 | 497 | 537 | 448 |
| Tandem | 58 | 45 | 63 | 61 | 61 | 173 |
| duplication | | | | | | |
| Unpaired | 134 | 62 | 125 | 308 | 228 | 124 |
| inversion | | | | | | |
| Inversion | 92 | 72 | 74 | 94 | 60 | 68 |
| Intra-chr. | 48 | 23 | 44 | 44 | 58 | 40 |
| insertion | | | | | | |
| Inter-chr. | 10 | 8 | 10 | 12 | 14 | 15 |
| insertion | | | | | | |
| Isolated | 45 | 25 | 49 | 43 | 59 | 50 |
| translocation | | | | | | |
| Balanced | 0 | 2 | 8 | 0¹⁾ | 4 | 0²⁾ |
| translocation | | | | | | |
| Complex | 209 | 56 | 147 | 150 | 197 | 152 |
| intra-chr. | | | | | | |
| Complex | 225 | 76 | 181 | 235 | 337 | 83 |
| inter-chr. | | | | | | |
| Total | 1,255 | 782 | 1,211 | 1,444 | 1,555 | 1,153 |

¹⁾BCR-ABL1 translocation but not the reciprocal ABL1-BCR is present due to deletion of derivative chromosome 9
²⁾BCR-ABL1 translocation but not the reciprocal ABL1-BCR is present due loss of derivative chromosome 9 and/or complex rearrangements.

TABLE E3
Filtering of predicted SVs by DNA-PET in 5 patient samples and K562

| | SV¹⁾ | Non-redundant²⁾ SV¹⁾ |
|---|---|---|
| Raw data | 7,400 | 3,408 |
| CML specific³⁾ | 1,301 | 1,220 |
| After quality filter⁴⁾ | 495 | 459 |

¹⁾SV statistics are reflected by numbers of dPET clusters (inversions, insertions, and balanced translocations are composed of two dPET clusters per event)
²⁾The same SV in different genomes is counted once.
³⁾After filtering the data by DNA-PET information of 23 normal libraries of 22 normal individuals and published paired-end sequencing data of normal samples (Kidd et al. 2008; Korbel et al. 2007).
⁴⁾dPET clusters were filtered which had a supercluster size >100, or a Blast score >2000, or a Blast alignment type of EC, or a cluster size of 2 (see Methods)

TABLE E4
CML specific SVs predicted by DNA-PET in 5 patient
samples and K562 after quality filtering

| | P440 | P145 | P308 | P098 | P022 | K562 |
|---|---|---|---|---|---|---|
| Deletion | 17 | 17 | 30 | 30 | 51 | 71 |
| Tandem duplication | 5 | 4 | 3 | 6 | 3 | 104 |
| Unpaired inversion | 5 | 3 | 3 | 16 | 8 | 26 |
| Inversion | 2 | 0 | 0 | 2 | 2 | 2 |
| Intra-chr. insertion | 0 | 0 | 1 | 1 | 0 | 0 |
| Inter-chr. insertion | 0 | 0 | 0 | 0 | 0 | 0 |
| Isolated translocation | 7 | 0 | 2 | 7 | 5 | 11 |
| Balanced translocation | 0 | 2 | 2 | 0¹ | 2 | 0² |
| Complex intra-chr. | 7 | 1 | 2 | 1 | 2 | 14 |
| Complex inter-chr. | 2 | 3 | 2 | 0 | 2 | 9 |
| Total | 45 | 30 | 45 | 63 | 75 | 237 |

¹BCR-ABL translocation but not the reciprocal ABL1-BCR is present due to deletion of derivative chromosome 9
²BCR-ABL translocation but not the reciprocal ABL1-BCR is present due to loss of derivative chromosome 9 and/or complex rearrangements

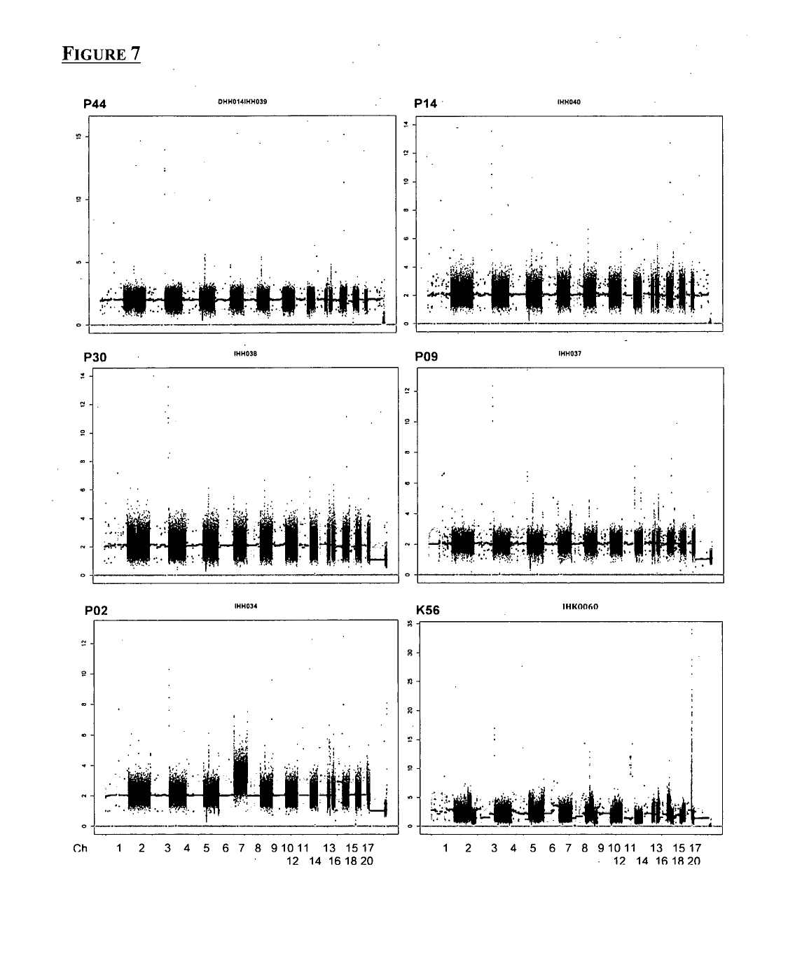

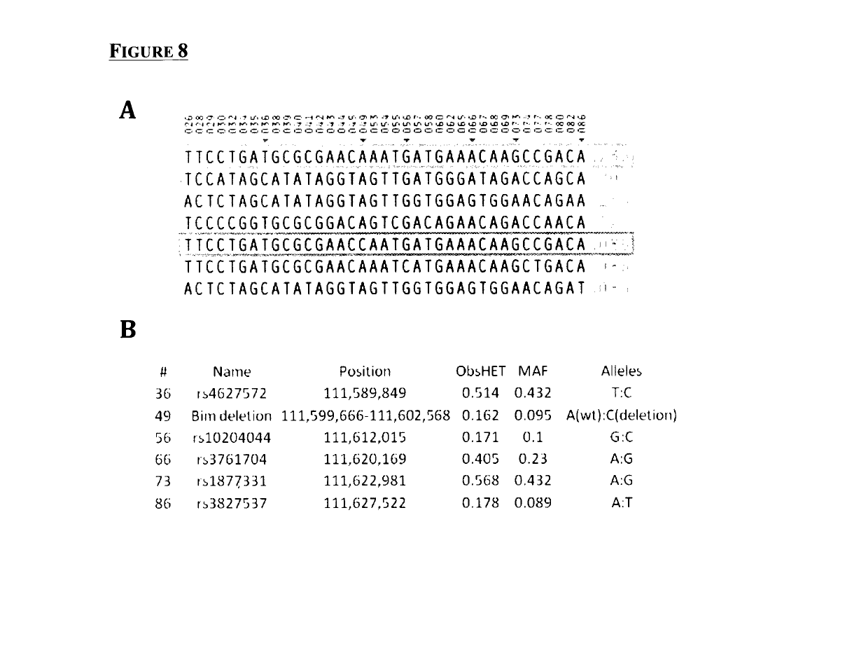

In order to exclude additional SVs that exist in the normal population, as well as decrease the proportion of false positives, the SVs were further filtered by additional DNA-PET data obtained from 32 normal and unrelated individuals, as well as by the following bioinformatics-defined quality criteria. Clusters were excluded if (i) they were interconnected with 100 or more other clusters indicated by a supercluster size >100, (ii) they had a high sequence similarity between the two joined breakpoint regions indicated by a Blast score >2000 between the two anchor regions including their extensions by 15 Kb towards the breakpoints, (iii) they showed a Blast score >300 between the two anchor/extension regions where the sequence similarity is within the anchor on one side and within the extension on the other side (EC type), (iv) they had a cluster size of 2. This resulted in 459 CML-specific SVs (Table E3 and Table E4) as well as copy number information (FIG. 7), and allowed us to generate karyo-genomic maps for each genome (FIG. 2B).

Importantly, using DNA-PE T, we were able to identify the BCR-ABL translocation in all but the remission sample. This dataset was then used to identify SVs that tracked with either disease-stage or drug-resistance.

### Example 8

### Results: DNA-PET Analysis of Blast Transformation

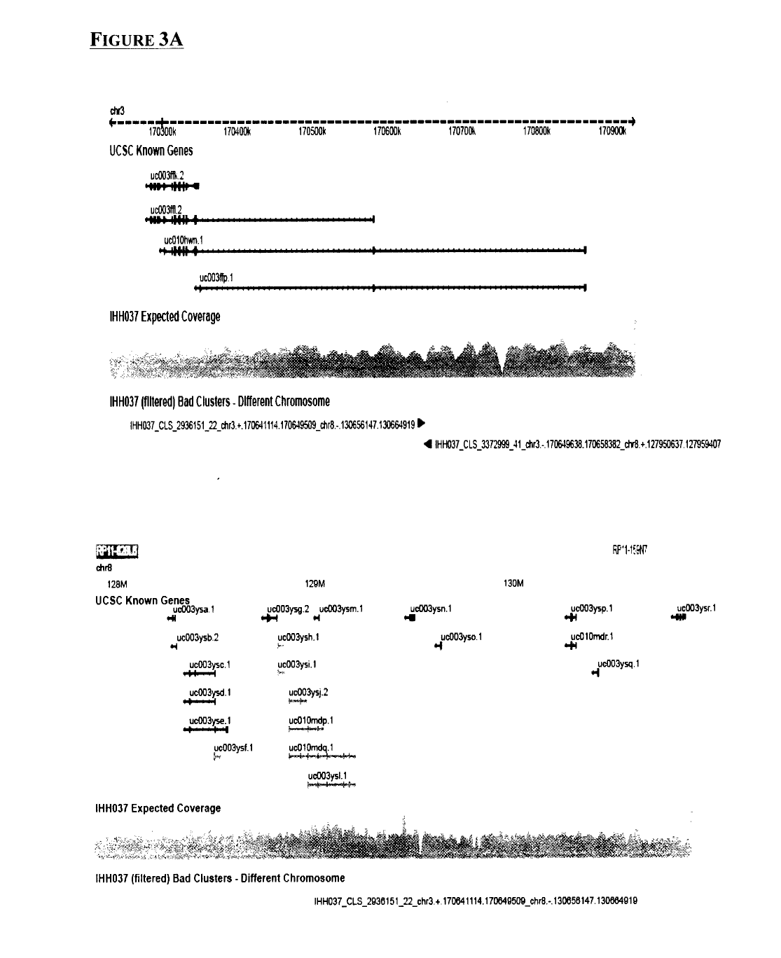

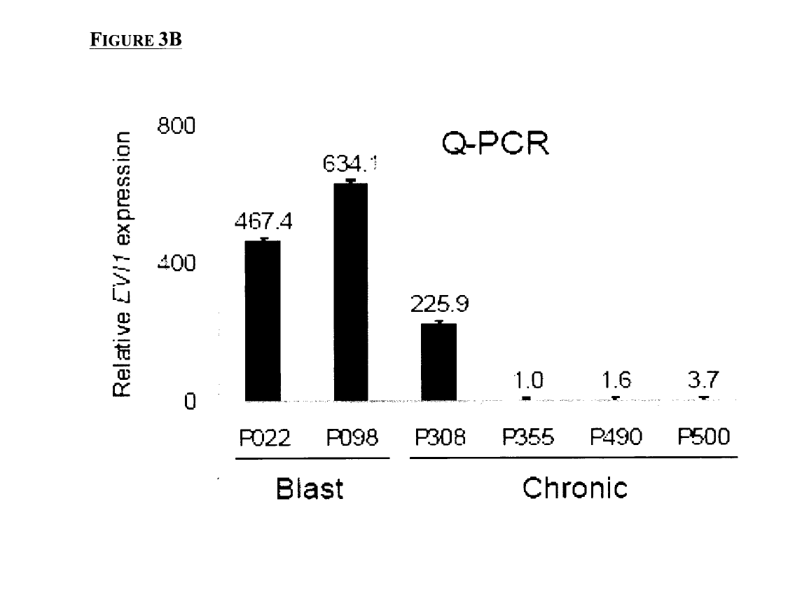

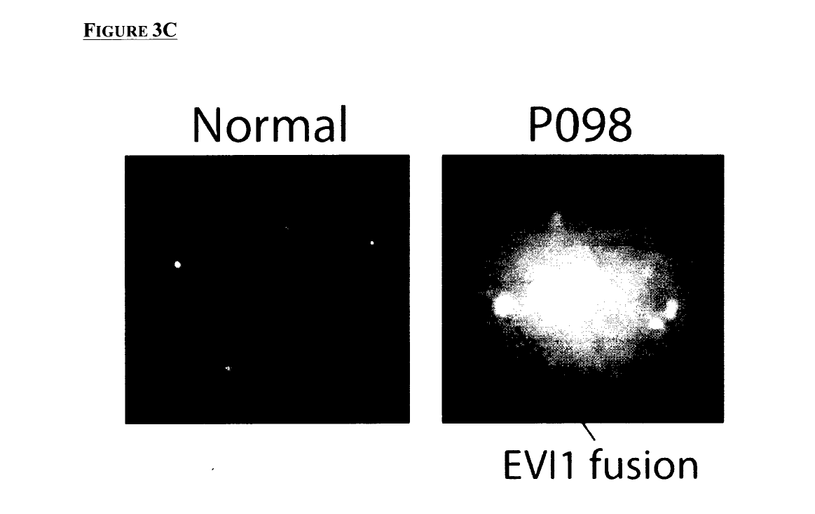

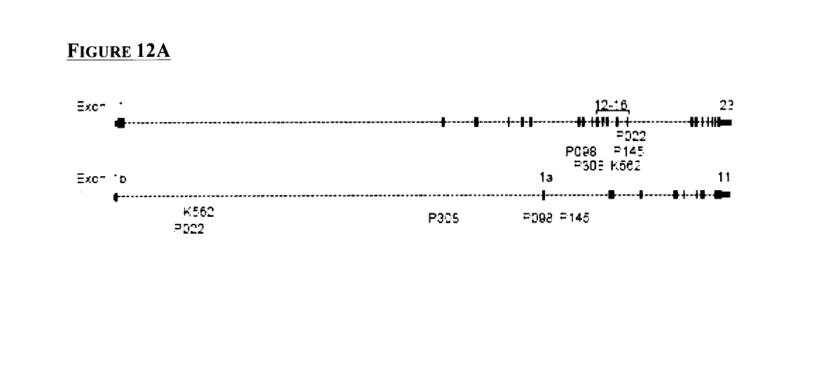

The additional chromosomal abnormalities and molecular aberrations that are found in blast phase are thought to contribute to clinical behavior. However, a comprehensive assessment of either the number or structural nature of these events has been technically challenging, particularly for the detection of SVs which are copy number neutral. Accordingly, we turned our attention to the analysis of SVs that occurred upon development of blast phase. Here, as depicted in Table E4, we saw a progressive increase in the number of SVs in blast phase, which correlated well with karyotyping and FISH analysis (Table E7 and FIGS. 3D and 11). We then categorized the nature of the SVs, and found that deletions are the most prominent category of SVs in the two blast phase patients (P098 and P022, n=81 [58.7% of all SVs]), followed by unpaired inversions (n=24 [17.4%]), isolated translocations (n=12 [8.7%]) and tandem duplications (n=9 [6.5%]) and less than 5% for each of the other SV categories (FIG. 2A). The blast phase cell line K562 showed, as expected, more rearrangements compared to the blast phase patient samples (237 vs. 63 and 75, respectively). Tandem duplications were the most prominent category in K562 (n=104 [43.9%), followed by deletions (n=71 [30%]), unpaired inversions (n=26 [11%]) and complex intra-chromosomal rearrangements (n=14 [5.9%]). Interestingly, no balanced translocations in addition to BCR-ABL1 were observed in the two blast phase samples and the K562 cell line.

TABLE E7
Validation of cytogenetic karyotypes by DNA-PET

| Cytogenetics | DNA-PET |
|---|---|
| P022 | |
| 46,XY,+8 | 3 copies of chr8 estimated on sequence based copy |
| | number (FIG. 7) |
| t(9;22)(q34;q11.2) | BCR-ABL1 translocation (FIG. 2B) |
| i(17)(q10)[cp2] | a dPET cluster of size 2 indicates isochromosome 17 |
| | rearrangement between pos. 17,595,066 and |
| | 28,282,853 (FIG. 11) |
| 48,idem + der(22)t(9;22)[7] | copy number of 2.7 upstream of chr22:21,955,000 |
| | and downstream of chr9:132,605,000 (points of copy |
| | number change in FIG. 7) |
| 48,idem + 19[cp4] | no detection of higher copy number of chr 19 |
| 48idem,t(12;17)(p13;q11.2) + 19[cp3] | not detected |
| 49,idem,+8,+19[2] | 3 copies of chr8 estimated on sequence based copy |
| | number (FIG. 7) |
| 46,XY[3] | |
| P098 | |
| 46,XY, | BCR-ABL1 translocation (FIG. 2B) |
| t(9;22)(q34;q11.2)[cp20] | |
| P145 | |
| 46,XY, | BCR-ABL1 translocation (FIG. 2B) |
| t(9;22)(q34;q11.2)[cp20] | |
| P440 | |
| 46,XY, | |
| P308 | |
| 46,XY, | BCR-ABL1 translocation (FIG. 2B) |
| t(9;22)(q34;q11.2)[cp20] | |

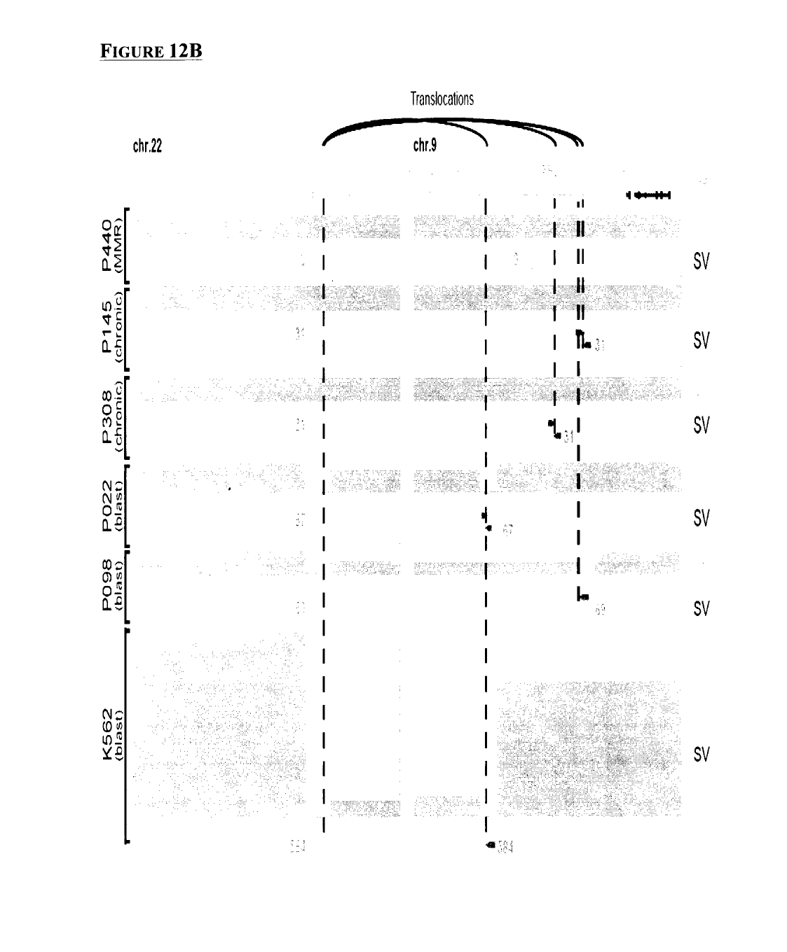

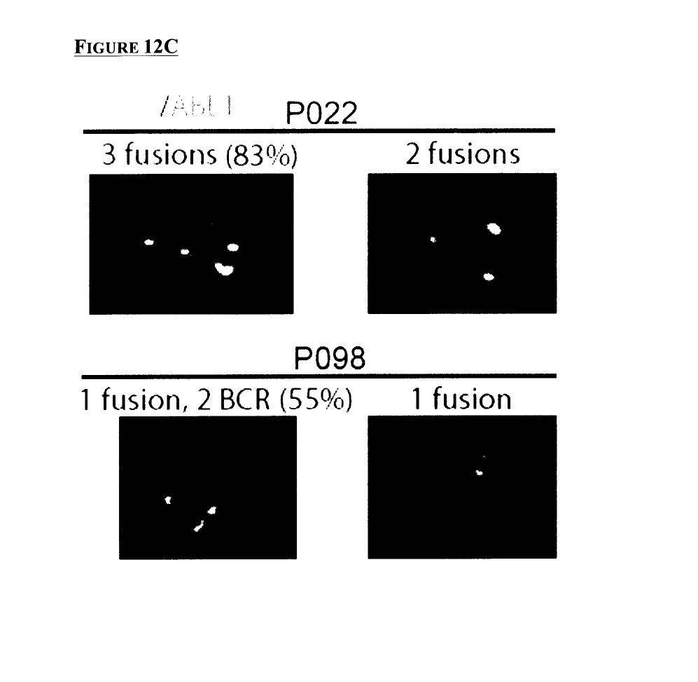

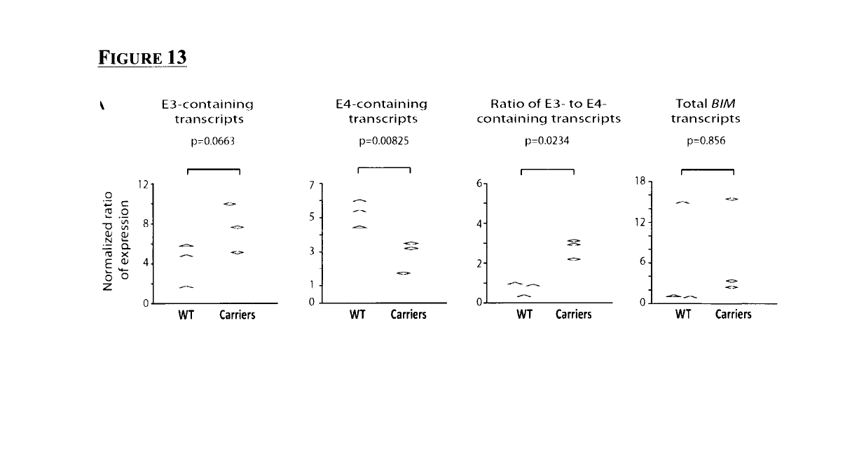

We next explored which of the individual somatic SVs could contribute biologically to blast phase by intersecting the blast phase specific SVs with RefSeq genes downloaded from the UCSC Genome Bioinformatics homepage (http://genome.ucsc.edu/) (Rhead et al. 2010), and identified 205 candidate SVs predicted to affect gene function directly. Within this group, we observed the amplification of BCR-ABL1 itself, which was appreciable as an increase in the dPET cluster size, and is a recognized feature of transformation (FIG. 12).^{6,10} We also observed an inverse correlation between the cluster size representing the reciprocal ABL1-BCR translocation and stage (FIG. 12). Of note, DNA-PET detected the complete loss of ABL1-BCR in sample P098, consistent with a deletion in the der9 by FISH (FIG. 12). Another candidate we identified was a 2.7 Mb insertion of chromosome 8 into intron 1 of MECOM (previously known as EVIL and MDS1) on chromosome 3 (FIG. 3A) which we confirmed by FISH (FIG. 3C). EVI-1 is a zinc finger transcription factor and plays an essential role in the proliferation and maintenance of normal hematopoietic stem cells (HSC)¹¹⁻¹³, and when overexpressed in HSCs results in bone marrow hyperproliferation and myeloid differentiation block. To our knowledge, no comparable insertion within or upstream of EVI1 has been reported, and analysis of the insertion site suggested that the insertion of chromosome 8 might alter the transcription level of the shorter transcripts, which we confirmed by RT-PCR (FIG. 3B).

Taken together, our DNA-PET analysis has been able to identify both known and novel features of blast phase progression, and provides the first assessment of the number and nature of structural changes that occur in a human cancer model.

### Example 9

### Results: An East-Asian BIM Polymorphism in Imatinib-Resistant Samples

We next investigated resistance-associated SVs, and found two deletions that occurred in all three resistant samples and six SVs in at least two out of three. One deletion, 2.5 Kb in size, was located in intron 1 of ZNF385D, a gene that has not yet been connected to CML. Interestingly, the other deletion observed in all three resistant samples was in intron 2 of the pro-apoptotic gene BCL2L11 (also known as BO D, BIM L, BIMEL, or BIM) (FIG. 4A). Importantly, others have reported that BIM upregulation by imatinib is required for the drug to induce apoptosis, since preventing BIM upregulation renders CML imatinib-resistant.¹⁴⁻¹⁶ We confirmed the deletion by PCR and sequencing, and found an identical 2,903 bp deletion in all 3 samples (FIG. 4C and FIG. 4D), suggesting that the deletion is germline and thus constitutes a polymorphism. Accordingly, we screened 74 normal East-Asian samples from the International HapMap Project,¹⁷ and determined the frequency of BIM deletion carriers to be 17.6% (12 heterozygous and 1 homozygous individual; allele frequency of 9.5%). Although the BIM deletion turned out to be a structural polymorphism rather than a recurrent somatic event, we were intrigued by the fact that all three resistant patients were carriers of the BIM deletion, a gene that is required for imatinib-sensitivity.¹⁴⁻¹⁶

### Example 10

### Results: Functional Effects of the BIM Polymorphism

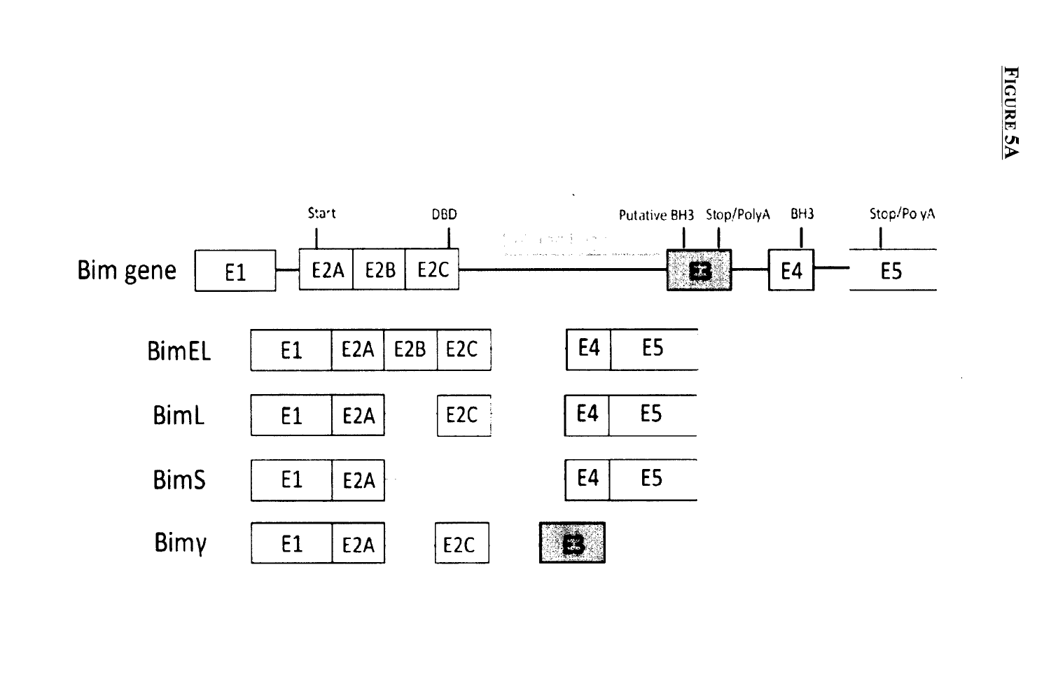

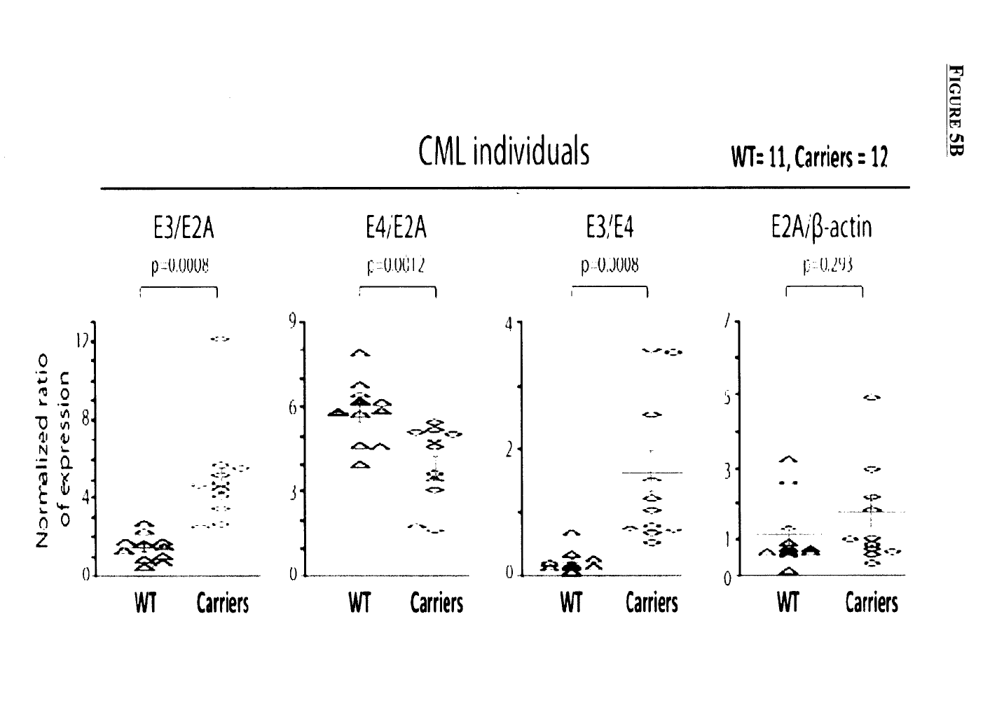

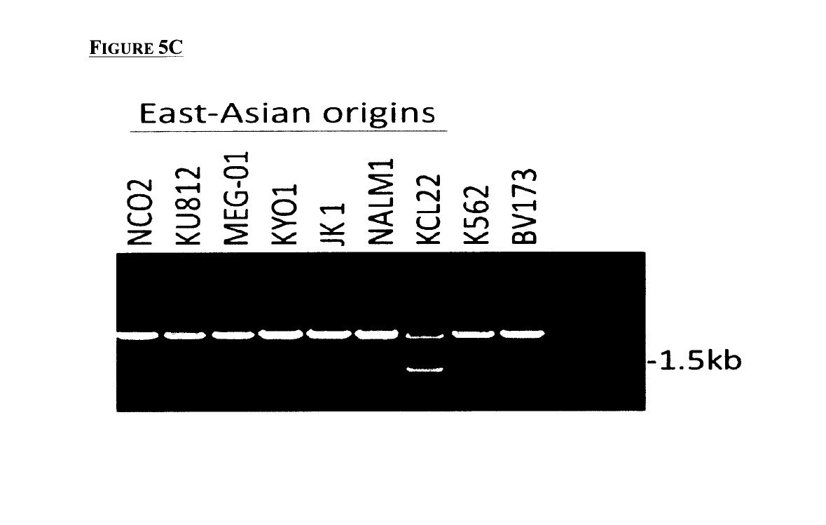

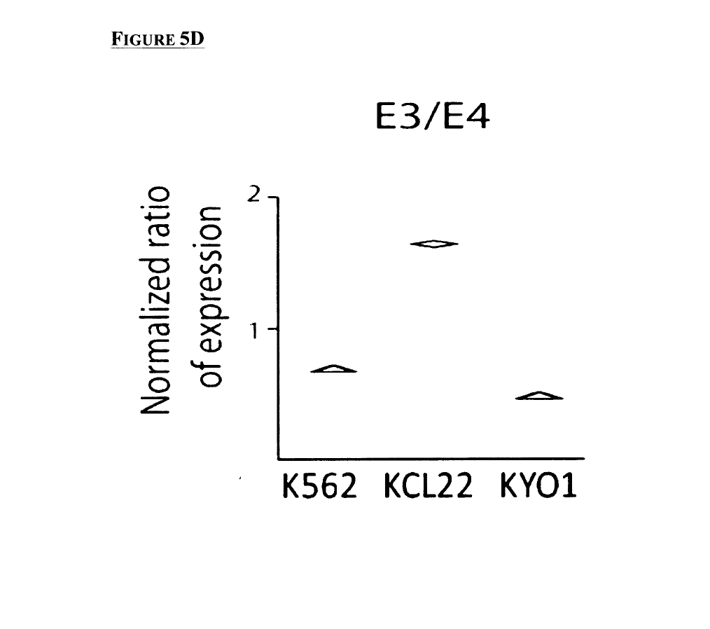

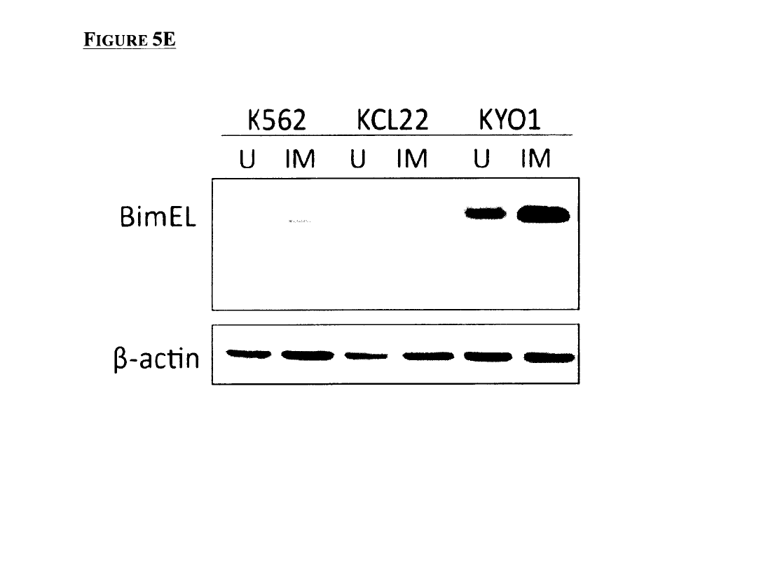

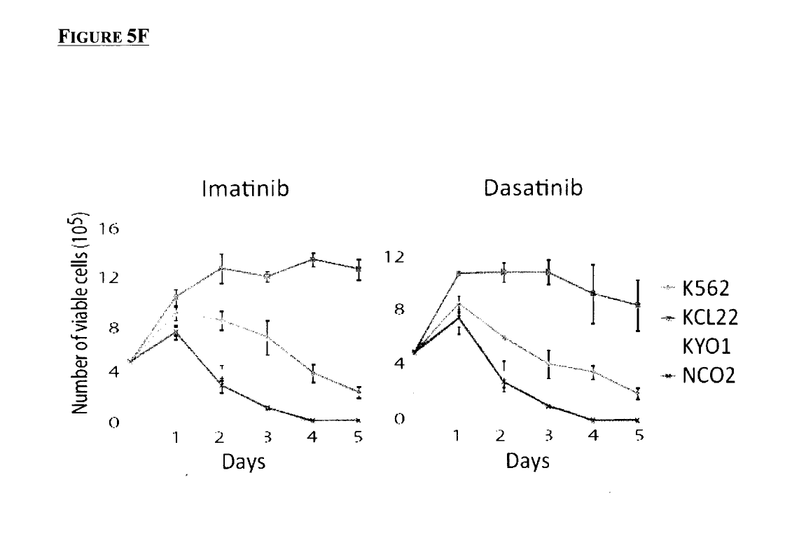

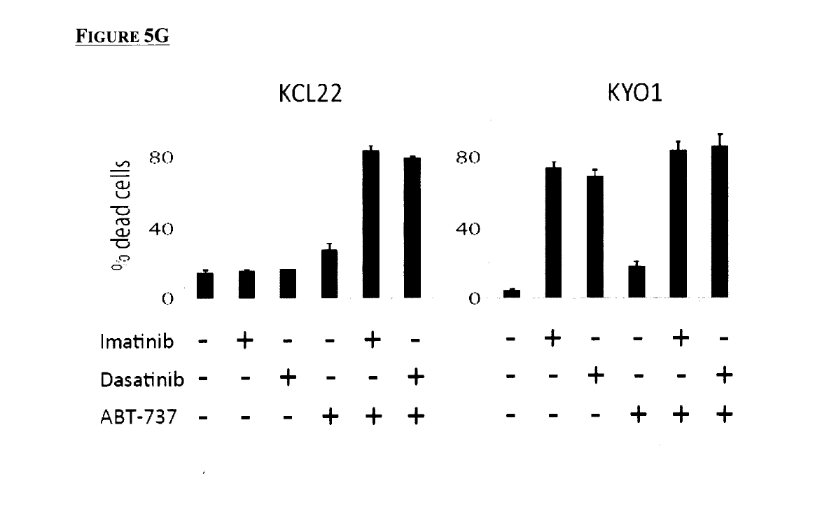

Analysis of BIM gene structure suggested that the deletion might result in alternative splicing of exon 3 and 4 in a mutually exclusive manner. This possibility was suggested by the deletion's proximity (107 bp) to the 5′ end of the intron-exon boundary of exon 3, as well as the presence of a stop codon within exon 3 itself (FIG. 5A). To test this hypothesis, we obtained primary CML samples with (n=12) and without (n=11) the deletion, and measured the expression levels of exon 3- and exon 4-containing transcripts, as well as exon 2-containing transcripts as a readout for general BIM transcription (since exon 2 is present in all BIM isoforms),¹⁸ As shown in FIG. 5B, we found that the deletion was associated with an increase in exon 3-containing transcripts together with a decrease in exon 4-containing transcripts, while general BIM transcription was unaffected. These results were mirrored in lymphoblastoid cell lines from normal individuals, indicating that the effect of the deletion is lineage-independent (FIG. 13).

Because the proapoptotic properties of BIM reside in the BH3 domain, which is found only in exon 4 (FIG. 5A),^{18,19} we asked if the decrease in exon 4-containing transcripts might be associated with imatinib-resistance. Fortunately, we were able to identify one CML cell line, KCL22, which harbored the deletion and used these cells to address this question (FIG. 5C). Notably, this line was originally obtained from a Japanese patient, and in contrast to most other CML lines, exhibited imatinib-resistance ab initio.²⁰⁻²² We confirmed that the cells expressed an increased exon 3/exon 4 transcript ratio (FIG. 5D) and that this was associated with decreased protein expression of the major exon 4-containing BIM isoform, BIMEL, in KCL22 cell lines both before and after imatinib exposure, when compared to lines without the deletion (FIG. 5E). We also confirmed that KCL22 cells were resistant to imatinib, as well as the more potent second-generation tyrosine kinase inhibitors (FIG. 5F).

These results led us to ask if BH3 mimetics, which are in early phase clinical trials,²³ might sensitize KCL22 cells to imatinib. As shown in FIG. 5G, we found that this was indeed the case, and that the combination of imatinib or dasatinib and ABT-737 acted synergistically to induce apoptosis in KCL22 cells but not KYO-1 cells.

### Example 11

### Results: Association of the BIM Polymorphism with BCR-ABL Independent Clinical Resistance to Tyrosine Kinase Inhibitors in Chronic Myelogenous Leukaemia

We next determined the frequency of the polymorphism in a larger cohort of East-Asian CML patients, and found it to be present in 12.0% (19 out of 158). We also tested the hypothesis that the polymorphism could predict for clinical resistance. In this analysis, we reasoned that the presence of the polymorphism, as exemplified by KCL22 cells, might be sufficient to confer resistance to tyrosine kinase inhibitors, whereas in its absence, resistance would require the emergence of clones bearing resistance-conferring BCR-ABL mutations.²⁴

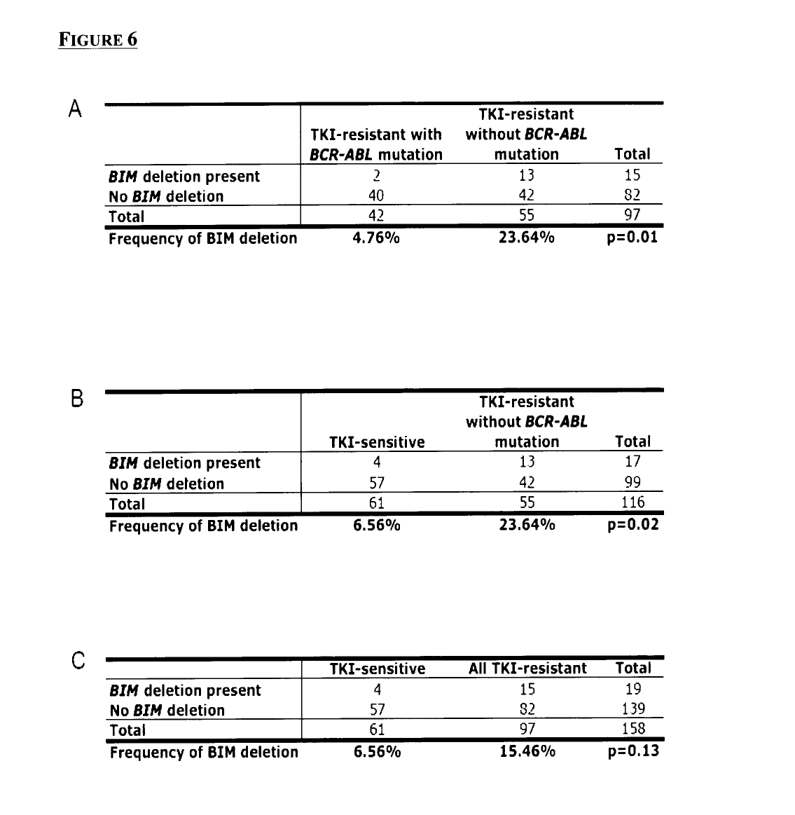

Accordingly, we divided the samples from all patients with resistance into those with or without BCR-ABL mutations, and found a significant difference in the frequency of the polymorphism between the two groups: 2 out of 42 (4.8%) with BCR-ABL mutations vs 13 out of 55 (23.6%) without (p=0.01) (FIG. 6A).

When we determined the frequency of the polymorphism in patients with sensitive vs resistant disease in the absence of a BCR-ABL mutation, the difference was also significant: 4 out of 61 in imatinib-sensitive patients versus 13 out of 55 for imatinib-resistant patients (p=0.02) (FIG. 6B). However, there was no significant difference in the frequency of the polymorphism between imatinib-sensitive patients (4 out of 61), and all imatinib-resistant patients (15 out of 97) (p=0.13) (FIG. 6C).

This result might be expected, given that statistical significance here would depend on the frequency of the BIM polymorphism in the general CML population.

### Example 12

### Discussion

We report a novel polymorphism in intron 2 of the BIM gene that is associated with both in vitro and in vivo clinical resistance to tyrosine kinase inhibitors. The frequency of the deletion carriers in the normal East-Asian population is 17.6%, and in the CML patients seen at two South-East Asian referral centers, 12.0%. In this cohort, the polymorphism accounted for a quarter (13/55 or 23.6%) of the cases with resistance in the absence of a BCR-ABL mutation. We believe an etiologic role is unlikely given the lack of geographical variations in the worldwide incidence of CML.²⁵

We also show that the presence of the polymorphism results in the increased expression of transcripts containing exon 3 vs exon 4 of the BIM gene, and found a significant association between the development of clinical resistance and the presence of the polymorphism, such that individuals with the deletion had a 1.34-fold relative risk (heterozygous genotype relative risk; 95% confidence interval 0.94-1.59) of developing resistance than those without. In addition, by determining that resistance is independent of BCR-ABL inhibition, we were able to predict that patients with resistance and the deletion would be less likely to harbor CML clones with resistance-conferring mutations in BCR-ABL than those without the deletion. This finding is consistent with the idea that such clones only emerge under the selective pressure of therapy, and in the absence of other resistance-conferring mechanisms.²⁴

Current clinical guidelines advocate either increasing the dose of tyrosine kinase inhibitor or changing to more potent tyrosine kinase inhibitors upon development of resistance.⁵ Hence it is of concern that among 4 patients with BIM polymorphism-associated resistance, and in whom these guidelines were followed and subsequent response data available, none responded. While anecdotal, this observation is consistent with our finding that BIM deletion-associated resistance is BCR-ABL-independent. However, our in vitro observations also suggest that resistance in this setting can be overcome by combining tyrosine kinase inhibitors with BH3 mimetics. Thus, the BIM polymorphism may be used as a predictor of both resistance to tyrosine kinase inhibitors and of response to BH3 mimetics. Screening for this polymorphism may therefore be useful in the management of East-Asian CML patients. The applicability of our findings to other cancers where drug-sensitivity is dependent on BIM-mediated apoptosis also warrants investigation.²⁶⁻²⁸

### Examples 13 and 14

### Association of the BIM Polymorphism with EGFR Independent Clinical Resistance to Tyrosine Kinase Inhibitors in Non-Small Cell Lung Cancer (NSCLC)

It has previously been shown that mutant EGFR-driven NSCLCs require BIM expression for TKIs to be able to kill NSCLC cell lines (Cragg et al. PLOS Medicine, 2007; Costa et al. PLOS Medicine, 2007).

Accordingly, we predict that cell lines, and by extension patients, bearing the BIM polymorphism will be resistant to drugs which target the mutant EGFR.

In addition, we predict that resistant cell lines will be sensitive to the combination of BH3 mimetic drugs (e.g. ABT-737 and ABT-263) and the anti-EGFR drug.

### Example 13

### Association of the BIM Polymorphism with EGFR Independent Clinical Resistance to Tyrosine Kinase Inhibitors in Non-Small Cell Lung Cancer (NSCLC)—in vitro Correlations

NSCLC cell lines with activating mutations in EGFR will be identified.

Cell lines will then be analyzed for the presence or absence of the BIM polymorphism by PCR analysis as previously described.

The sensitivity and/or resistance of the various cell lines to anti-EGFR drugs will be determined using standard cellular assays for cell growth, proliferation, and apoptosis.

Cell lines will also be tested for their sensitivity to BH3 mimetics, either as single agents or in combination with drugs targeting EGFR.

The presence or absence of the polymorphism will then be correlated with the degree of sensitivity/resistance of the NSCLC cell line to the drugs being tested.

We expect that there will be a positive correlation between the presence of the BIM polymorphism and drug-resistance.

Specifically, we predict that NSCLC lines with activating mutations in EGFR bearing the BIM polymorphism will be more resistant to anti-EGFR drugs than those not bearing the BIM polymorphism.

Furthermore, we expect that cell lines with the BIM polymorphism and which are resistant to drugs targeting EGFR will be sensitive to the combination of BH3 mimetic drugs (e.g. ABT-737 and ABT-263) and the anti-EGFR drug.

### Example 14

### Association of the BIM Polymorphism with EGFR Independent Clinical Resistance to Tyrosine Kinase Inhibitors in Non-Small Cell Lung Cancer (NSCLC)—Patient Correlations

Patients with NSCLC with activating mutations in EGFR will be identified.

DNA from patients and/or their tumours will be analyzed for the presence or absence of the BIM polymorphism by PCR analysis as previously described.

The patient's response to the anti-EGFR therapy will then be determined using clinical criteria. These data will include clinical parameters such as tumour size, time to progression, progression free survival, overall survival, and performance status.

The presence or absence of the polymorphism will then be correlated with the clinical parameters mentioned above.

We expect that there will be a positive correlation between the presence of the BIM polymorphism and an inferior response to anti-EGFR therapy compared to patients without the polymorphism. The may be seen as a failure of the tumour to decrease in size.

In addition, such patients may have a shorter time to progression, progression free survival, and/or overall survival.

### Examples 15 and 16

### Association of the BIM Polymorphism with c-KIT/PDGFR Independent Clinical Resistance to Tyrosine Kinase Inhibitors in Gastrointestinal Stromal Tumours (GIST)

It has previously been shown that c-KIT/PDGFR-driven GISTs require BIM expression for TKIs to be able to kill GIST cell lines (Gordon et al. JBC, 2010).

Accordingly, we predict that cell lines, and by extension patients, bearing the BIM polymorphism will be resistant to drugs which target the mutant c-KIT.

In addition, we predict that resistant cell lines will be sensitive to the combination of BH3 mimetic drugs (e.g. ABT-737 and ABT-263) and the anti-c-KIT drug.

### Example 15

### Association of the BIM Polymorphism with c-KIT/PDGFR Independent Clinical Resistance to Tyrosine Kinase Inhibitors in Gastrointestinal Stromal Tumours (GIST)—in vitro Correlations

c-KIT-driven GIST cell lines with activating mutations in c-KIT will be identified.

Cell lines will then be analyzed for the presence or absence of the BIM polymorphism by PCR analysis as previously described.

The sensitivity and/or resistance of the various cell lines to anti-c-KIT drugs will be determined using standard cellular assays for cell growth, proliferation, and apoptosis.

Cell lines will also be tested for their sensitivity to BH3 mimetics, either as single agents or in combination with drugs targeting c-KIT.

The presence or absence of the polymorphism will then be correlated with the degree of sensitivity/resistance of the GIST cell line to the drugs being tested.

We expect that there will be a positive correlation between the presence of the BIM polymorphism and drug-resistance.

Specifically, we predict that GIST lines with activating mutations in c-KIT bearing the BIM polymorphism will be more resistant to anti-c-KIT drugs than those not bearing the BIM polymorphism.

Furthermore, we expect that cell lines with the BIM polymorphism and which are resistant to drugs targeting c-KIT will be sensitive to the combination of BH3 mimetic drugs (e.g. ABT-737 and ABT-263) and the anti-c-KIT drug.

### Example 16

### Association of the BIM Polymorphism with c-KIT/PDGFR Independent Clinical Resistance to Tyrosine Kinase Inhibitors in Gastrointestinal Stromal Tumours (GIST)—Patient Correlations

Patients with GIST with activating mutations in c-KIT will be identified.

DNA from patients and/or their tumours will be analyzed for the presence or absence of the BIM polymorphism by PCR analysis as previously described.

The patient's response to the anti-c-KIT therapy will then be determined using standard clinical criteria. These data will include clinical parameters such as tumour size, time to progression, progression free survival, overall survival, and performance status.

The presence or absence of the polymorphism will then be correlated with the clinical parameters mentioned above.

We expect that there will be a positive correlation between the presence of the BIM polymorphism and an inferior response to anti-c-KIT therapy compared to patients without the polymorphism. The may be seen as a failure of the tumour to decrease in size. In addition, such patients may have a shorter time to progression, progression free survival, and/or overall survival.

### References for Examples 1 to 16

1. Hochhaus A, O'Brien S G, Guilhot F, et al. Six-year follow-up of patients receiving imatinib for the first-line treatment of chronic myeloid leukemia. Leukemia 2009; 23(6):1054-61.
2. Druker B J, Sawyers C L, Kantarjian H, et al. Activity of a specific inhibitor of the BCR-ABL tyrosine kinase in the blast crisis of chronic myeloid leukemia and acute lymphoblastic leukemia with the Philadelphia chromosome. The New England journal of medicine 2001; 344(14):1038-42.
3. Sokal J E, Cox E B, Baccarani M, et al. Prognostic discrimination in “good-risk” chronic granulocytic leukemia. Blood 1984; 63(4):789-99.
4. Hasford J, Pfirrmann M, Hehlmann R, et al. A new prognostic score for survival of patients with chronic myeloid leukemia treated with interferon alfa. Writing Committee for the Collaborative CML Prognostic Factors Project Group. Journal of the National Cancer Institute 1998; 90(11):850-8.
5. Baccarani M, Cortes J, Pane F, et al. Chronic myeloid leukemia: an update of concepts and management recommendations of European LeukemiaNet. J Clin Oncol 2009; 27(35):6041-51.
6. Calabretta B, Perrotti D. The biology of CML blast crisis. Blood 2004; 103(11):4010-22.
7. Quintas-Cardama A, Cortes J. Molecular biology of bcr-abl1-positive chronic myeloid leukemia. Blood 2009; 113(8):1619-30.
8. Oehler V G, Yeung K Y, Choi Y E, Bumgarner R E, Raftery A E, Radich J P. The derivation of diagnostic markers of chronic myeloid leukemia progression from microarray data. Blood 2009; 114(15):3292-8.
9. Fullwood M J, Wei C L, Liu E T, Ruan Y. Next-generation DNA sequencing of paired-end tags (PET) for transcriptome and genome analyses. Genome research 2009; 19(4):521-32.
10. Deininger M W, Goldman J M, Melo J V. The molecular biology of chronic myeloid leukemia. Blood 2000; 96(10):3343-56.
11. Goyama S, Kurokawa M. Pathogenetic significance of ecotropic viral integration site-1 in hematological malignancies. Cancer science 2009; 100(6):990-5.
12. Mitani K. Molecular mechanisms of leukemogenesis by AML1/EVI-1. Oncogene 2004; 23(24):4263-9.
13. Jolkowska J, Witt M. The EVI-1 gene—its role in pathogenesis of human leukemias. Leukemia research 2000; 24(7):553-8.
14. Aichberger K J, Mayerhofer M, Krauth M T, et al. Low-level expression of proapoptotic Bcl-2-interacting mediator in leukemic cells in patients with chronic myeloid leukemia: role of BCR/ABL, characterization of underlying signaling pathways, and reexpression by novel pharmacologic compounds. Cancer research 2005; 65(20):9436-44.
15. Kuroda J, Puthalakath H, Cragg M S, et al. Bim and Bad mediate imatinib-induced killing of Bcr/Abl+leukemic cells, and resistance due to their loss is overcome by a BH3 mimetic. Proceedings of the National Academy of Sciences of the United States of America 2006; 103(40):14907-12.
16. Kuribara R, Honda H, Matsui H, et al. Roles of Bim in apoptosis of normal and Bcr-Abl-expressing hematopoietic progenitors. Molecular and cellular biology 2004; 24(14):6172-83.
17. The International HapMap Project. Nature 2003; 426(6968):789-96.
18. Adachi M, Zhao X, Imai K. Nomenclature of dynein light chain-linked BH3-only protein Bim isoforms. Cell death and differentiation 2005; 12(2):192-3.
19. Strasser A, Puthalakath H, Bouillet P, et al. The role of bim, a proapoptotic BH3-only member of the Bcl-2 family in cell-death control. Annals of the New York Academy of Sciences 2000; 917:541-8.
20. Kubonishi I, Miyoshi I. Establishment of a Ph1 chromosome-positive cell line from chronic myelogenous leukemia in blast crisis. International journal of cell cloning 1983; 1(2):105-17.
21. Mahon F X, Deininger M W, Schultheis B, et al. Selection and characterization of BCR-ABL positive cell lines with differential sensitivity to the tyrosine kinase inhibitor STI571: diverse mechanisms of resistance. Blood 2000; 96(3):1070-9.
22. Deininger M W, Goldman J M, Lydon N, Melo J V. The tyrosine kinase inhibitor CGP57148B selectively inhibits the growth of BCR-ABL-positive cells. Blood 1997; 90(9):3691-8.
23. Cragg M S, Harris C, Strasser A, Scott C L. Unleashing the power of inhibitors of oncogenic kinases through BH3 mimetics. Nature reviews 2009; 9(5):321-6.
24. Wodarz D, Komarova N L. Emergence and prevention of resistance against small molecule inhibitors. Seminars in cancer biology 2005; 15(6):506-14.
25. Druker B J, Lee S J. Chronic Myelogenous Leukemia. In: DeVita V T, Lawrence T S, Rosenberg S A, eds. Cancer Principles & Practice of Oncology. 8th ed. Philadelphia, Pa. 19106 USA: Lippincott Williams & Wilkins; 2008:2267-78.
26. Cragg M S, Kuroda J, Puthalakath H, Huang D C, Strasser A. Gefitinib-induced killing of NSCLC cell lines expressing mutant EGFR requires BIM and can be enhanced by BH3 mimetics. PLoS medicine 2007; 4(10):1681-89; discussion 90.
27. Costa D B, Halmos B, Kumar A, et al. BIM mediates EGFR tyrosine kinase inhibitor-induced apoptosis in lung cancers with oncogenic EGFR mutations. PLoS medicine 2007; 4(10):1669-79; discussion 80.
28. Gordon P M, Fisher D E. Role for the proapoptotic factor BIM in mediating imatinib-induced apoptosis in a c-KIT-dependent gastrointestinal stromal tumor cell line. The Journal of biological chemistry; 285(19):14109-14.
29. Krzywinski M, Schein J, Birol I, et al. Circos: an information aesthetic for comparative genomics. Genome Res 2009; 19(9):1639-45.
30. Adachi M, Zhao X, Imai K. Nomenclature of dynein light chain-linked BH3-only protein Bim isoforms. Cell Death Differ 2005: 12(2):192-3.
31. Liu J W, Chandra D, Tang S H, Chopra D, Tang DG. Identification and characterization of Bimgamma, a novel proapoptotic BH3-only splice variant of Bim. Cancer Res 2002 62(10):2976-81.
32. Fujita P A, Rhead B, Zweig A S, Hinrichs A S, Karolchik D, Cline M S, Goldman M, Barber G P, Clawson H, Coelho A, Diekhans M, Dreszer T R, Giardine B M, Harte R A, Hillman-Jackson J, Hsu F, Kirkup V, Kuhn R M, Learned K, Li C H, Meyer L R, Pohl A, Raney B J, Rosenbloom K R, Smith K E, Haussler D, Kent W J. The UCSC Genome Browser database: update 2011. Nucleic Acids Res. 2010 Oct. 18. [Epub ahead of print]
A1. Baccarani, M., G. Saglio, J. Goldman, A. Hochhaus, B. Simonsson, F. Appelbaum, J. Apperley, F. Cervantes, J. Cortes, M. Deininger, A. Gratwohl, F. Guilhot, M. Horowitz, T. Hughes, H. Kantarjian, R. Larson, D. Niederwieser, R. Silver, and R. Hehlmann. 2006. Evolving concepts in the management of chronic myeloid leukemia: recommendations from an expert panel on behalf of the European LeukemiaNet. Blood 108:1809-20.
A2. Fullwood, M. J., C. L. Wei, E. T. Liu, and Y. Ruan. 2009. Next-generation DNA sequencing of paired-end tags (PET) for transcriptome and genome analyses. Genome Res 19:521-32.
A3. Ruan, Y., H. S. Ooi, S. W. Choo, K. P. Chiu, X. D. Zhao, K. G. Srinivasan, F. Yao, C. Y. Choo, J. Liu, P. Ariyaratne, W. G. Bin, V. A. Kuznetsov, A. Shahab, W. K. Sung, G. Bourque, N. Palanisamy, and C. L. Wei. 2007. Fusion transcripts and transcribed retrotransposed loci discovered through comprehensive transcriptome analysis using Paired-End diTags (PETs). Genome Res 17:828-38.
A4. San Jose-Eneriz, E., X. Agirre, A. Jimenez-Velasco, L. Cordeu, V. Martin, V. Arqueros, L. Garate, V. Fresquet, F. Cervantes, J. A. Martinez-Climent, A. Heiniger, A. Torres, F. Prosper, and J. Roman-Gomez. 2009. Epigenetic down-regulation of BIM expression is associated with reduced optimal responses to imatinib treatment in chronic myeloid leukaemia. Eur J Cancer 45:1877-89.
A5. Kuribara, R., H. Honda, H. Matsui, T. Shinjyo, T. Inukai, K. Sugita, S. Nakazawa, H. Hirai, K. Ozawa, and T. Inaba. 2004. Roles of Bim in apoptosis of normal and Bcr-Abl-expressing hematopoietic progenitors. Mol Cell Biol 24:6172-83.
A6. Kuroda, J., H. Puthalakath, M. S. Cragg, P. N. Kelly, P. Bouillet, D. C. Huang, S. Kimura, O. G. Ottmann, B. J. Druker, A. Villunger, A. W. Roberts, and A. Strasser. 2006. Bim and Bad mediate imatinib-induced killing of Bcr/Abl+ leukemic cells, and resistance due to their loss is overcome by a BH3 mimetic. Proc Natl Acad Sci USA 103:14907-12.
A7. Belloc, F., F. Moreau-Gaudry, M. Uhalde, L. Cazalis, M. Jeanneteau, F. Lacombe, V. Praloran, and F. X. Mahon. 2007. Imatinib and nilotinib induce apoptosis of chronic myeloid leukemia cells through a Bim-dependant pathway modulated by cytokines. Cancer Biol Ther 6:912-9.
A8. Tse, C., A. R. Shoemaker, J. Adickes, M. G. Anderson, J. Chen, S. Jin, E. F. Johnson, K. C. Marsh, M. J. Mitten, P. Nimmer, L. Roberts, S. K. Tahir, Y. Xiao, X. Yang, H. Zhang, S. Fesik, S, H. Rosenberg, and S. W. Elmore. 2008. ABT-263: a potent and orally bioavailable Bcl-2 family inhibitor. Cancer Res 68:3421-8.
A9. Hendrickson, A. W., X. W. Meng, and S. H. Kaufmann. 2008. Anticancer therapy: boosting the bang of Bim. J Clin Invest 118:3582-4.
A10. Kang, M. H., and C. P. Reynolds. 2009. Bcl-2 inhibitors: targeting mitochondrial apoptotic pathways in cancer therapy. Clin Cancer Res 15:1126-32.
A11. Gillings, A. S., K. Balmanno, C. M. Wiggins, M. Johnson, and S. J. Cook. 2009. Apoptosis and autophagy: BIM as a mediator of tumour cell death in response to oncogene-targeted therapeutics. Febs J 276:6050-62.

A12. Kuroda, J., and M. Taniwaki. 2009. Involvement of BH3-only proteins in hematologic malignancies. Crit Rev Oncol Hematol 71:89-101.
A13. Del Gaizo Moore, V., J. R. Brown, M. Certo, T. M. Love, C. D. Novina, and A. Letai. 2007. Chronic lymphocytic leukemia requires BCL2 to sequester prodeath BIM, explaining sensitivity to BCL2 antagonist ABT-737. J Clin Invest 117:112-21.
A14. Leung, K. T., K. K. L1, S. S. Sun, P. K. Chan, V. E. Ooi, and L. C. Chiu. 2008. Activation of the JNK pathway promotes phosphorylation and degradation of BimEL—a novel mechanism of chemoresistance in T-cell acute lymphoblastic leukemia. Carcinogenesis 29:544-51.
A15. Rambal, A. A., Z. L. Panaguiton, L. Kramer, S. Grant, and H. Harada. 2009. MEK inhibitors potentiate dexamethasone lethality in acute lymphoblastic leukemia cells through the pro-apoptotic molecule BIM. Leukemia 23:1744-54.
A16. Nordigarden, A., M. Kraft, P. Eliasson, V. Labi, E. W. Lam, A. Villunger, and J. I. Jonsson. 2009. BH3-only protein Bim more critical than Puma in tyrosine kinase inhibitor-induced apoptosis of human leukemic cells and transduced hematopoietic progenitors carrying oncogenic FLT3. Blood 113:2302-11.
A17. De Bruyne, E., T. J. Bos, F. Schuit, E. Van Valckenborgh, E. Menu, L. Thorrez, P. Atadja, H. Jernberg-Wiklund, and K. Vanderkerken. 2010. IGF-1 suppresses Bim expression in multiple myeloma via epigenetic and posttranslational mechanisms. Blood.
A18. Aichberger, K. J., K. V. Gleixner, I. Mirkina, S. Cerny-Reiterer, B. Peter, V. Ferenc, M. Kneidinger, C. Baumgartner, M. Mayerhofer, A. Gruze, W. F. Pickl, C. Sillaber, and P. Valent. 2009. Identification of proapoptotic Bim as a tumor suppressor in neoplastic mast cells: role of KIT D816V and effects of various targeted drugs. Blood 114:5342-51.
A19. Costa, D. B., B. Halmos, A. Kumar, S. T. Schumer, M. S. Huberman, T. J. Boggon, D. G. Tenen, and S. Kobayashi. 2007. BIM mediates EGFR tyrosine kinase inhibitor-induced apoptosis in lung cancers with oncogenic EGFR mutations. PLoS Med 4:1669-79; discussion 1680.
A20. Cragg, M. S., J. Kuroda, H. Puthalakath, D. C. Huang, and A. Strasser. 2007. Gefitinib-induced killing of NSCLC cell lines expressing mutant EGFR requires BIM and can be enhanced by BH3 mimetics. PLoS Med 4:1681-89; discussion 1690.
A21. Gong, Y., R. Somwar, K. Politi, M. Balak, J. Chmielecki, X. Jiang, and W. Pao. 2007. Induction of BIM is essential for apoptosis triggered by EGFR kinase inhibitors in mutant EGFR-dependent lung adenocarcinomas. PLoS Med 4:e294.
A22. de La Motte Rouge, T., L. Galluzzi, K. A. Olaussen, Y. Zermati, E. Tasdemir, T. Robert, H. Ripoche, V. Lazar, P. Dessen, F. Harper, G. Pierron, G. Pinna, N. Araujo, A. Harel-Belan, J. P. Armand, T. W. Wong, J. C. Soria, and G. Kroemer. 2007. A novel epidermal growth factor receptor inhibitor promotes apoptosis in non-small cell lung cancer cells resistant to erlotinib. Cancer Res 67:6253-62.
A23. Deng, J., T. Shimamura, S. Perera, N. E. Carlson, D. Cai, G. I. Shapiro, K. K. Wong, and A. Letai. 2007. Proapoptotic BH3-only BCL-2 family protein BIM connects death signaling from epidermal growth factor receptor inhibition to the mitochondrion. Cancer Res 67:11867-75.
A24. Hann, C. L., V. C. Daniel, E. A. Sugar, I. Dobromilskaya, S. C. Murphy, L. Cope, X. Lin, J. S. Hierman, D. L. Wilburn, D. N. Watkins, and C. M. Rudin. 2008. Therapeutic efficacy of ABT-737, a selective inhibitor of BCL-2, in small cell lung cancer. Cancer Res 68:2321-8.
A25. L1, D. D., X. Q. Wu, J. Tang, X. Y. Wei, and X. F. Zhu. 2009. ON-III inhibits erbB-2 tyrosine kinase receptor signal pathway and triggers apoptosis through induction of Bim in breast cancer cells. Cancer Biol Ther 8:739-43.
A26. Stan, S. D., E. R. Hahm, R. Warin, and S. V. Singh. 2008. Withaferin A causes FOXO3a- and Bim-dependent apoptosis and inhibits growth of human breast cancer cells in vivo. Cancer Res 68:7661-9.
A27. Okumura, K., S. Huang, and F. A. Sinicrope. 2008. Induction of Noxa sensitizes human colorectal cancer cells expressing Mcl-1 to the small-molecule Bcl-2/Bcl-xL inhibitor, ABT-737. Clin Cancer Res 14:8132-42.
A28. Zhang, W., G. Z. Cheng, J. Gong, U. Hermanto, C. S. Zong, J. Chan, J. Q. Cheng, and L. H. Wang. 2008. RACK1 and CIS mediate the degradation of BimEL in cancer cells. J Biol Chem 283:16416-26.
A29. Esteve-Puig, R., F. Canals, N. Colome, G. Merlino, and J. A. Recio. 2009. Uncoupling of the LKB1-AMPKalpha energy sensor pathway by growth factors and oncogenic BRAF. PLoS One 4:e4771.
A30. Fontana, L., M. E. Fiori, S. Albini, L. Cifaldi, S. Giovinazzi, M. Forloni, R. Boldrini, A. Donfrancesco, V. Federici, P. Giacomini, C. Peschle, and D. Fruci. 2008. Antagomir-17-5p abolishes the growth of therapy-resistant neuroblastoma through p21 and BIM. PLoS One 3:e2236.
A31. Kubonishi, I., and I. Miyoshi. 1983. Establishment of a Ph1 chromosome-positive cell line from chronic myelogenous leukemia in blast crisis. Int J Cell Cloning 1:105-17.
Barrett J C, Fry B, Maller J, Daly M J (2005) Haploview: analysis and visualization of LD and haplotype maps. Bioinformatics 21: 263-5
Kidd J M, Cooper G M, Donahue W F, Hayden H S, Sampas N, Graves T, Hansen N, Teague B, Alkan C, Antonacci F, Haugen E, Zerr T, Yamada N A, Tsang P, Newman T L, Tuzun E, Cheng Z, Ebling H M, Tusneem N, David R, Gillett W, Phelps K A, Weaver M, Saranga D, Brand A, Tao W, Gustafson E, McKernan K, Chen L, Malig M, Smith J D, Korn J M, McCarroll S A, Altshuler D A, Peiffer D A, Dörschner M, Stamatoyannopoulos J, Schwartz D, Nickerson D A, Mullikin J C, Wilson R K, Bruhn L, Olson M V, Kaul R, Smith D R, Eichler EE (2008) Mapping and sequencing of structural variation from eight human genomes. Nature 453: 56-64
Korbel J O, Urban A E, Affourtit J P, Godwin B, Grubert F, Simons J F, Kim P M, Palejev D, Carriero N J, Du L, Taillon B E, Chen Z, Tanzer A, Saunders A C, Chi J, Yang F, Carter N P, Hurles M E, Weissman S M, Harkins T T, Gerstein M B, Egholm M, Snyder M (2007) Paired-end mapping reveals extensive structural variation in the human genome. Science 318: 420-6

### Examples H1 to H8

Examples H1 to H8 demonstrate that a common deletion polymorphism in the BIM gene contributes to intrinsic resistance to imatinib in chronic myelogenous leukemia.

### Example H1

### Materials and Methods

Ethics Committee Approval

Clinical CML samples were obtained from patients seen at the Singapore General Hospital, Akita University Hospital, the University of Malaya Medical Centre, and the National University Cancer Institute, Singapore. German control samples were obtained from blood donors at the University Hospital of Bonn. Malay, Chinese, and Indian control samples were derived from recent local population studies^{4,5}. Written informed consent and institutional review board approval at the participating institutions were obtained from all patients and normal individuals who contributed samples to this study.

Mapping of Sequence Tags

The paired tags were mapped individually to the reference sequence (NCBI build 36) in color space allowing 2 color code mismatches per tag by the SOLiD System Analysis Pipeline Tool Corona Lite (Applied Biosystems). Contigs of the reference sequence with unresolved location (random_chr) and alternative MHC haplotypes were excluded from the reference for mapping. Individually mapped tags were paired by Corona Lite. In cases where one or both tags had multiple mapping locations, a process termed ‘rescuing’ favored the creation of concordant PETs (both tags are on the same chromosome, same strand, same orientation, correct 5′→3′ order and in the expected distance to each other).

Clustering of Discordant PETs (dPETs)

Paired-end ditags (PETs) were categorized as concordant PETs (cPETs) where both tags mapped to same chromosome, same strand, in the correct 5′ to 3′ ordering and within expected span range. The span range was determined based on the gradient of the span distribution as described in Hillmer et al¹. The PETs which were rejected by cPET criteria were classified as discordant PETs (dPETs). To cluster different dPETs which span the same fusion point, the following procedure was applied: the mapping location of the 5′ and 3′ tags of a given dPET was extended by the maximum insert size of the respective genomic library in both directions, creating 5′ and 3′ windows. If the 5′ and 3′ tags of a second dPET mapped within the 5′ and 3′ window of the first dPET, the two PETs were defined as a cluster of the size 2 and the 5′ and 3′ windows were adjusted so that they contained the tag extensions (by the maximum library size) of the second dPET. Subsequently, dPETs which mapped with their 5′ and 3′ tags within the 5′ and 3′ windows, respectively, were assigned to this cluster and the windows were adjusted, if necessary. The number of dPETs clustering together around a fusion point was represented by the cluster size. The genomic region which was covered by the 5′ tags of a cluster was defined as the 5′ anchor and the genomic region which was covered by the 3′ tags of a cluster was defined as the 3′ anchor. dPET clusters with anchor regions <500 bp were excluded from further analysis.

The DNA-PET sequencing of K562 has been described earlier¹. The same K562 dPET clusters have been used in the present study but the centromeric regions have not been excluded (as has been done in the previous study), dPET clusters with anchor regions <500 bp were excluded (previously <1000 bp), and a new exclusion/filtering procedure has been applied (see below).

Exclusion of Low Confidence Clusters

We excluded low confidence clusters from further analysis using a series of statistical tests. The tests determine library-specific thresholds for dPET clusters that can arise from chimeric sequence constructs or local insert size variation. In particular, as dPET clusters indicating short deletions can arise as an artifact of non-uniform span distribution, we use a binomial model for the distribution of “stretched” dPETs (discordant only because the ends are more than maximum library-size apart) on the genome (discretized into bins of maximum library-size) to estimate a minimum cluster-size threshold, such that less than 1 false positive is expected on average (with p-value bonferroni-corrected by the number of genomic bins). Specifically, the threshold (applied only to clusters whose ends are less than twice the maximum library size apart) is computed as:

$${min}_{k} b * f ( k ; N , \frac{1}{b} ) < 1$$

where N is the number of stretched dPETs, b is the number of bins and f(k; n, p) is the binomial density function at k for n independent trials with success probability p.

Chimeric PET constructs arising from the circularization step in the library preparation are expected to be dispersed across the genome and therefore less likely to form dPET clusters. Due to the sheer volume of sequences, however, a few such clusters do arise and we employ a similar binomial model as before to set a library-specific minimum cluster size threshold (with less than 1 false positive expected on average):

$${min}_{k} {b}^{2} * f ( k ; D , \frac{1}{{b}^{2}} ) < 1$$

where D is the number of dPETs. Note that while stretched dPET clusters can be defined by a single bin (to a first approximation), in general, two bins have to be selected to define a dPET cluster and this explains the quadratic factor in the above equation. As chimeric PET clusters are less likely to arise such that the ends are in close proximity to each other, the above threshold is not applied to clusters whose ends are closer than:

$${min}_{t} \frac{G}{t} {b ( t )}^{2} f ( 2 ; D , \frac{1}{{b ( t )}^{2}} ) < 1$$

where G is the size of the genome and b(t) is the number of bins in a region of size t. Cluster size thresholds defined by this methodology are shown in Table H12.

Superclustering

Some loci showed an accumulation of dPET clusters. At these loci it might be misleading to assign a particular SV to a dPET cluster and we established a breakpoint based interconnection network (superclustering¹) to separate breakpoints in complex regions from isolated and less complex SVs. To determine the neighborhood of a breakpoint, the start and end points of each dPET cluster anchor region were extended by the maximum insert size of the respective genomic library as search windows. If windows of neighboring clusters overlapped with each other, dPET clusters were grouped together into a supercluster. The procedure allowed an indirect connection of cluster A via B to C. The number of dPET clusters that could be joined together into a supercluster was represented by the supercluster size. In cases where >3 dPET clusters were interconnected, the breakpoint pairs were classified as ‘complex’ (intra- and inter-chromosomal). This interconnection network was established after the exclusion of low confidence clusters (see above) but before DNA-PET data curation (see below).

DNA-PET Data Curation

The raw DNA-PET data predicted 3,408 different SVs in the six CML samples (Table H4).

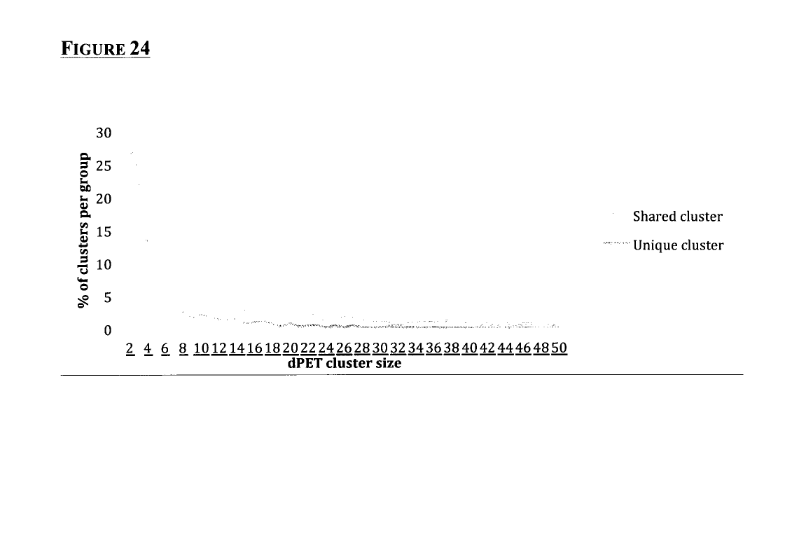

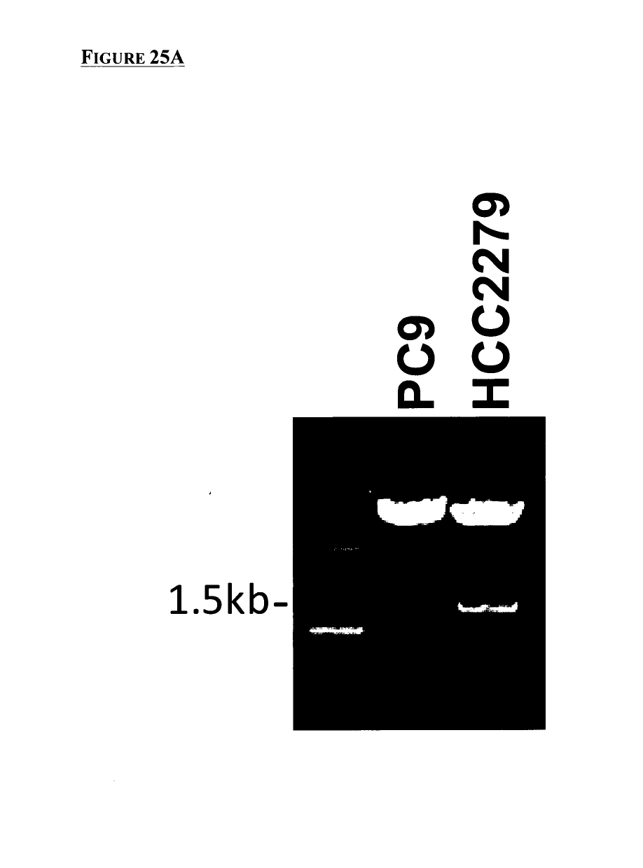

We filtered the data based on the following quality criteria: i) We compared the cluster sizes of the dPET clusters of the paired chronic phase/remission sample (P145 and P440) and divided the clusters in two groups: a) clusters which were identified in both samples and b) clusters which were only observed in one of the two. This stratification suggested that clusters of sizes 2 to 4 were enriched for artifacts (FIG. 24). To be conservative, we excluded all dPET clusters of size 2-5 from SV calling. ii) Mapping artifacts can create dPET clusters between regions with high sequence similarity. Although a procedure called ‘rescuing’ of the SOLiD pairing pipeline strongly reduces such artifacts by favoring the concordant pairing of two tags, sequence similarities between regions can still create artifacts. However, the exclusion of all dPET clusters with high sequence similarity is problematic since sequence similarity can trigger rearrangements by non-allelic homologous recombination. To evaluate the sequence similarity between the breakpoints, we extended the left and right anchor regions by 15 Kb toward the breakpoints and aligned by BLAST program (b12seq)⁶ the two regions. The alignment of two regions usually resulted in multiple alignments where we used the one with the highest score (defined as “BlastScore1” in Table H6).

In cases where the Blast score was >2,000, it was usually not possible to design specific PCR primers for validation and we therefore excluded dPET clusters with such high Blast scores. iii) In particular, sequence similarities between the anchor region on one side and the extension of the anchor on the other side of two predicted paired breakpoints is indicative for mapping artifacts (EC alignment type for ‘extension—cluster’). This is based on the assumption that the correct mapping of the anchor tags to the extension of the other anchor, with which they share sequence features, would create cPETs. We excluded dPET clusters with Blast scores >300 and an EC alignment type. iv) Some genomic regions tend to accumulate mapping artifacts and we excluded all dPET clusters with a supercluster size >100. We observed true rearrangements of such complexity only in cell lines but not in clinical samples¹ (and unpublished data). This filtering procedure resulted in 1,349 different predicted SVs in CML samples (Table H4).

Cross-Genome Comparison

Comparison of clusters across different genomes was performed based on an overlap of the 5′ and 3′ anchor regions extended by 10 Kb on both sides. If the 5′ anchor region of a cluster of a second library was overlapping with the 5′ extended anchor region of a cluster of the first library and the same was true for the 3′ anchor regions, the two clusters were grouped together and the 10 Kb extension of the anchor regions were adjusted according to the outermost start and end anchor coordinates. Breakpoint locations were used to compare the identified SVs with published SVs based on paired-end sequencing studies of non-cancer individuals^{7,8}. The fraction of a predicted SV which overlapped with a published SV was calculated by the percentage of overlap relative to the larger event. Gene annotations were based on RefSeq Genes downloaded from UCSC (http://genome.ucsc.edu/; 9) on May 14, 2009.

We included DNA-PET data of 21 normal individuals comprising of nine Chinese, six European, three African, two Mexican, and one Indian (22 libraries, unpublished data) in the cross-genome comparison. All SVs of the CML patients which matched SVs of one or more of the 21 normal samples have been categorized as germline SVs and were excluded from further analysis. In addition, SVs which overlapped by 80% or more with SVs which have been identified by paired-end sequencing approaches in ten non-cancer individuals comprising five African, three European, one Japanese, and one Chinese^{7,8} have been categorized as germline and were excluded. This ‘germline filtering procedure’ using 31 normal samples resulted in 309 different predicted SVs in CML samples (Tables H4 to H6). For validation of predicted SVs in CML genomes, PCR primers flanking the predicted breakpoints were used to amplify rearranged regions for subsequent Sanger sequencing.

Characterization of Blast Phase Rearrangements

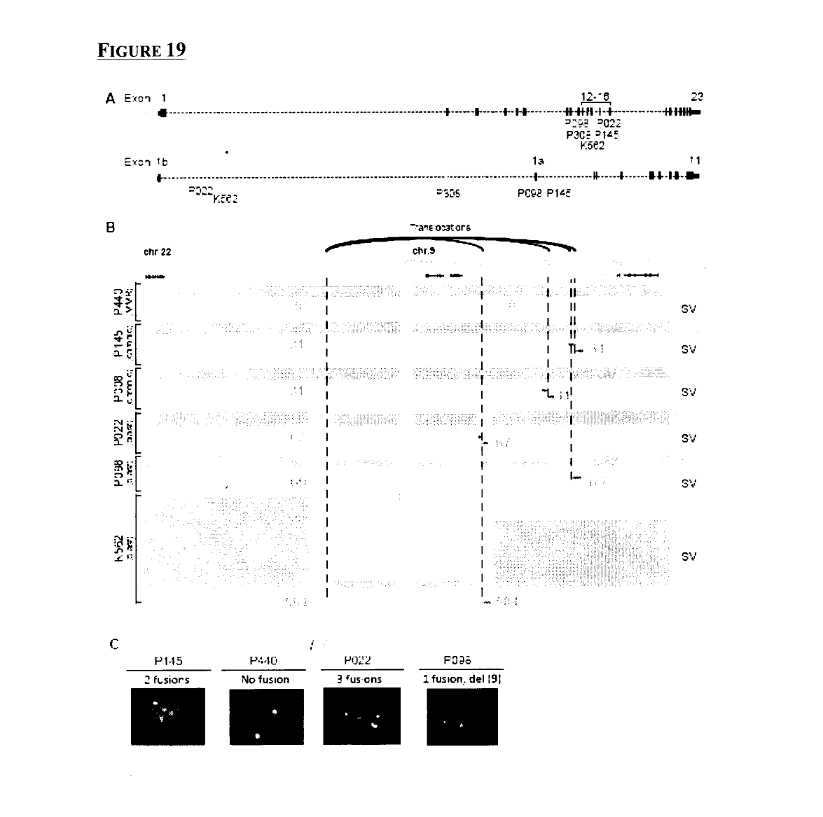

A consistent molecular finding in the transition to blast phase is an increase in levels of the BCR-ABL protein itself^{3,10,11}. This occurs via several mechanisms including BCR-ABL gene amplification and duplication of the Ph chromosome^{3,10,11}. Accordingly, we were able to detect an increase in the DNA-PET signal for BCR-ABL in the blast phase samples (P022, P098, K562) when compared to the two chronic phase samples (P145 and P308), which we correlated with FISH (FIG. 19B and FIG. 19C). We also observed an inverse correlation between the DNA-PET signal representing the reciprocal ABL-BCR translocation and stage (FIG. 19B). Deletions of the ABL-BCR rearrangement point on der9 have been described¹² and we detected the complete loss of ABL-BCR in blast samples P098 and K562 (FIGS. 19B and 19C) underlying the inverse correlation.

We categorized the nature of the potentially somatic SVs in the blast phase samples, and found that deletions were the most prominent category of SVs in the two blast phase patients (P098 and P022, n=79 [79% of all SVs]), followed by tandem duplications (n=6 [6%]) and isolated translocations (n=5 [5%]) and less than 5% for each of the other SV categories (Table H5).

TABLE H5
CML-specific SVs¹⁾ Predicted by DNA-PET in
Five Patient Samples and the K562 Cell Line

| | P440 | P145 | P308 | P098 | P022 | K562 |
|---|---|---|---|---|---|---|
| Deletion | 16 | 17 | 29 | 30 | 49 | 70 |
| Tandem duplication | 4 | 4 | 2 | 3 | 3 | 39 |
| Unpaired inversion | 3 | 2 | 0 | 0 | 2 | 20 |
| Inversion | 0 | 0 | 0 | 2 | 2 | 0 |
| Intra-chr. insertion | 0 | 0 | 0 | 0 | 0 | 0 |
| Inter-chr. insertion | 0 | 0 | 0 | 0 | 0 | 0 |
| Isolated translocation | 1 | 0 | 0 | 4 | 1 | 11 |
| Balanced translocation | 0 | 2 | 2 | 0²⁾ | 2 | 0³⁾ |
| Complex intra-chr. | 3 | 1 | 2 | 1 | 1 | 11 |
| Complex inter-chr. | 0 | 0 | 1 | 0 | 0 | 2 |
| Total | 27⁴⁾ | 26⁴⁾ | 36 | 40 | 60 | 153 |

¹⁾SV statistics are reflected by numbers of dPET clusters (inversions, insertions, and balanced translocations are composed of two dPET clusters per event)
²⁾BCR-ABL translocation but not the reciprocal ABL-BCR is present due to deletion of derivative chromosome 9
³⁾BCR-ABL translocation but not the reciprocal ABL-BCR is present due to loss of derivative chromosome 9 or complex rearrangements.
⁴⁾Four predicted SVs in the chronic phase patient sample P145 were not predicted in remission sample (P440) of the same patient (absence in remission) and seven SVs were predicted in P440 which had no DNA-PET indication in P145 (absence in chronic). In the ‘absence in remission’ category, two of the rearrangement points were BCR-ABL and ABL-BCR and two 5 Kb deletions have been missed in P440 but were identified by PCR in both samples. In the ‘absence in chronic’ category, a deletion and an isolated translocation could not be validated by PCR and have been excluded from the list, the five remaining discrepant SVs could be detected by PCR in both, the chronic and remission sample, and have been missed by DNA-PET in the chronic sample.

The blast phase cell line K562 showed, as expected, more rearrangements compared to the blast phase patient samples (153 vs. 40 and 60, respectively). Deletions were also the most prominent category in K562 (n=70 [46%), followed by tandem duplications (n=39 [25%]), unpaired inversions (n=20 [13%]), isolated translocations (n=11 [7%]), and complex intra-chromosomal rearrangements (n-11 [7%]). Interestingly, no balanced translocations in addition to BCR-ABL were observed in the two blast phase samples and the K562 cell line.

Cell Lines and Cell Culture Conditions

CML lines were obtained from the ATCC (MEG-01 and KU812), the Japanese Collection of Research Bioresources (NCO2), and the German Collection of Microorganisms and Cell Cultures (KCL22, K562, KYO-1, JK1, BV173, and NALM1). Cells were grown in RPMI-1640 medium supplemented with penicillin/streptomycin, glutamine and 10% FBS, and incubated in a humidified incubator at 37° C. with 5% CO₂.

Fluorescence In Situ Hybridization (FISH)

Nuclei were harvested by treating cells with 0.75M KCl for 15 min at 37° C. After fixation, nuclei were dropped on slides for FISH. BCR-ABL fusion was detected by the Vysis LSI (Locus specific identifier) BCR/ABL1 dual fusion translocation probe (Abbott Molecular). The LSI BCR probe is labeled with SpectrumGreen and the LSI ABL1 probe is labeled with SpectrumOrange. FISH assay using the LSI BCR/ABL1 dual color dual fusion translocation probe (Abbott Molecular) was performed on the fixed cells according to the manufacturer's instructions, with slight modifications. Briefly, the slide was dehydrated in an alcohol series and co-denaturation was carried out for 3 min at 75° C., followed by an overnight hybridization at 37° C. Evaluation of the FISH signals was performed using a fluorescence microscope (Olympus BX60) under 1000× magnification. For each case, about 200 interphase nuclei were evaluated for the signal pattern.

Real-Time RT-PCR

Total cellular RNAs were extracted using the RNeasy Mini Kit (Qiagen) according to the manufacturer's instruction. RNA was reverse transcribed using Superscript III First-Strand Synthesis System (Invitrogen), and quantitatively assessed using the iQ5 Multicolor Real-Time Detection System (Bio-Rad) with a total reaction volume of 25 μl. Primers were annealed at 59° C. for 20 seconds and the amplicon was extended at 72° C. for 30 seconds. The total number of cycles quantified was 40. Transcript levels of β-actin or exon 2A of BIM were used to normalize between samples. The following primers were used: BIM exon 2A (Forward: ATGGCAAAGCAACCTTCTGATG; Reverse: GGCTCTGTCTGTAGGGAGGT), BIM exon 3 (Forward: CAATGGTAGTCATCCTAGAGG; Reverse: GACAAAATGCTCAAGGAAGAGG), BIM exon 4 (Forward: TTCCATGAGGCAGGCTGAAC; Reverse: CCTCCTTGCATAGTAAGCGTT) and β-actin (Forward: GGACTTCGAGCAAGAGATGG; Reverse: AGCACTGTGTTGGCGTAC-AG).

Western Blot

Western blotting was performed using antibodies against human BIM, CrkL, pCrkL, CASPASE 3, Cleaved CASPASE 3, PARP (all from Cell Signaling Technology) and β-actin (Sigma). Detection was performed with HRP-conjugated secondary antibodies that are specific to rabbit (Sigma) or mouse IgG (Santa Cruz). The membrane was visualized using the Western Lightning chemiluminescence reagent (PerkinElmer).

Cell Death Detection by Annexin V Staining

Apoptosis was measured using the Annexin V-FITC/7-AAD kit (Beckman Coulter) according to the manufacturer's instructions. Briefly, treated and untreated cells were Annexin V-FITC- and 7-AAD-stained in 1× binding buffer for 15 min at room temperature and then analyzed by flow cytometry.

siRNA Knockdown of E3-Containing BIM Transcripts

Small interfering RNAs (siRNAs) against E3-containing BIM transcripts (BIMγ siRNA1: CCACCAUAGUCAAGAUACA; BIMγ siRNA2: CAGAACAACUCAACCACAA) and negative control siRNA (ON-TARGETplus Non-targeting siRNA #1) were purchased from Dharmacon Inc. Nucleofection was performed on KCL22 cells using Nucleofector Solution V (Lonza) in the presence of siRNAs.

Screening for the Deletion in BIM

We used Affymetrix Genome-Wide Human SNP Array 6.0 intensity data downloaded from the HapMap¹³ home page (http://snp.cshl.org/) to infer the copy number of the deletion polymorphism in BIM. Two genotyped single nucleotide positions were located within the deletion: SNP_A-4195083 and CN_{—}173550. The raw intensities of the two markers were used to call the copy number variation event using a Gaussian mixture model similar to the algorithm proposed by Korn and colleagues¹⁴. Using this procedure, we predicted the copy number and thereby the presence or absence of the deletion in unrelated HapMap samples of European (n=60), Yoruban (n=60), and Chinese/Japanese (n=90) origin. We then genotyped the deletion in Chinese/Japanese of which we had DNA samples (n=74) by PCR using primers Bim_del_F AATACCACAGAGGCCCACAG and Bim_del_R GCCTGAAGGTGCTGAGAAAG and JumpStart RedAccuTaq LA DNA Polymerase (Sigma) with the following thermo cycling conditions: 96° C. for 30sec, (94° C. for 15sec, 64° C. for 30sec, 68° C. for 5 min)×12, (94° C. for 15sec, 60° C. for 30sec, 68° C. for 5 min)×18, 68° C. for 20 min. The resulting PCR products with deletion (1,323 bp) and without the deletion (4,226 bp) were analyzed on 1% agarose gels. We used the PCR based genotypes to refine the single nucleotide intensity cutoffs for genotype calling in the European and Yoruban samples and used only the PCR validated genotypes of the East-Asian samples for frequency assessment.

To investigate further whether the deletion in the European population is at moderate frequency but has been missed by chance in the HapMap samples and to determine more precisely the deletion frequency in Asia, we genotyped by PCR assay 595 German, 600 Malay, 608 Chinese, and 605 Indian samples.

Calculation of Attributable Fractions for the BIM Deletion

To calculate the population attributable fraction of treatment resistance of East-Asian patients, we used PAF=(f(OR−1))/(f(OR−1)+1) with f, the frequency of deletion carriers among patients (f=0.135) and the odds ratio (OR) of the deletion carriers between patients being resistant and patients being sensitive to TKI treatment (OR=3.19).

Minigene Plasmid Construction

The pI-12 splicing construct was obtained as a generous gift from Mariano Garcia-Blanco. Using standard cloning techniques, two other minigene constructs, pI-12-WT and pI-12-MUT were constructed. Briefly, BIM exon 4, together with a 659 bp sequence upstream of exon 4, were amplified from KCL22 genomic DNA using 5′-GCCGCTCGAGTCTCTCCATGTGGTGTTTG-3′ as the forward primer and 5′-GCCGAAGCTTCCTCCTTGCATAGTAAGCGTT-3′ as the reverse primer. The PCR product was digested with XhoI and HindIII and cloned into the XhoI and HindIII sites in the pI-12 plasmid. BIM exon 3 and the upstream region with and without the deletion polymorphism was amplified from KCL22 genomic DNA using 5′-GCCGGATATCATGGAAGGAACTGACCTGGTG-3′ as the forward primer and 5′-GCCGATCGATGTAGGAAACTGGGTGAATGGC-3′ as the reverse primer. Two PCR products, with a size of 4500 bp and 1597 bp, were obtained and they were gel-purified, digested with EcoRV and ClaI and cloned into the EcoRV and ClaI sites in the plasmid to obtain the pI-12-WT construct that does not contain the deletion polymorphism and the pI-12-MUT construct containing the deletion polymorphism.

Zinc Finger Nucleases (ZFN) Used to Genome-Edit the BIM Deletion Polymorphism

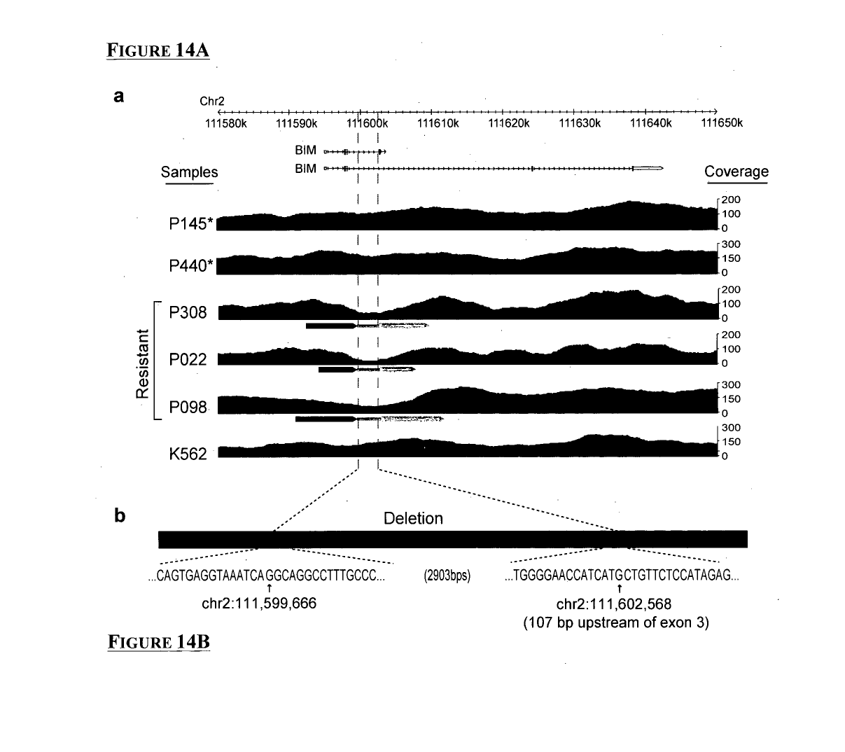

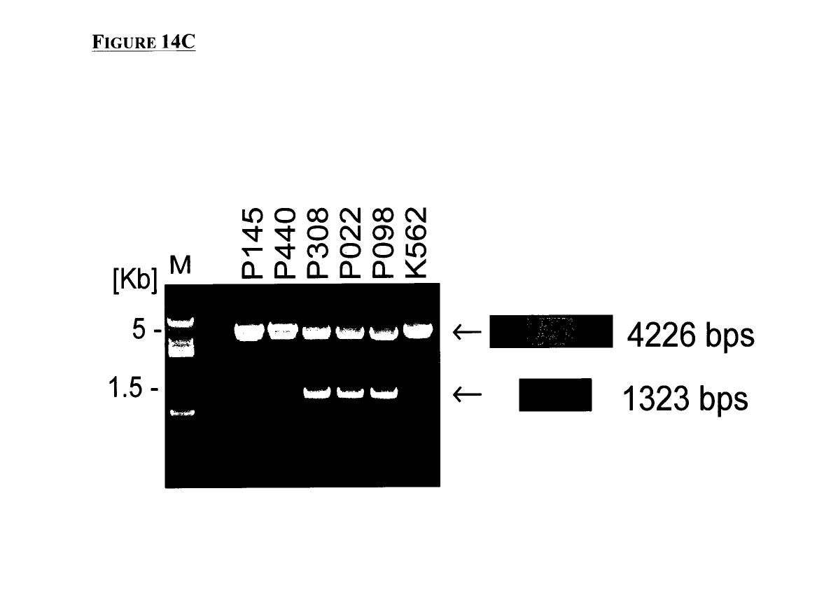

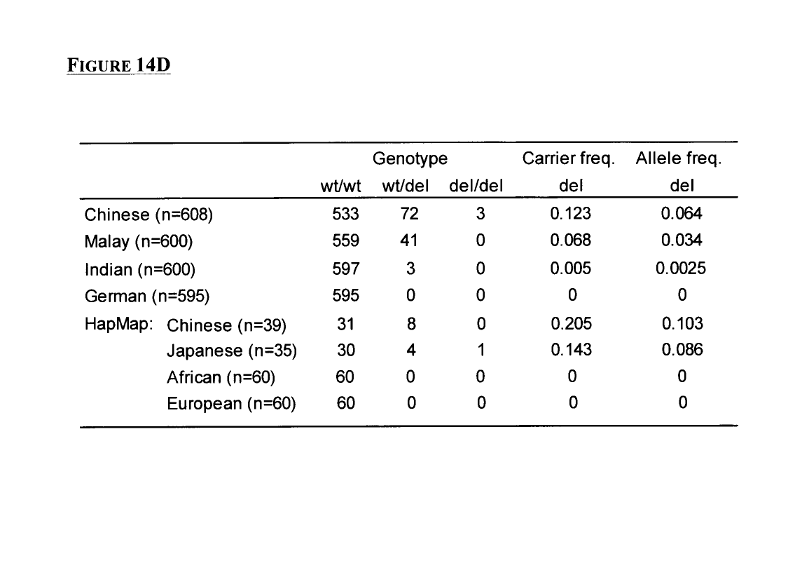

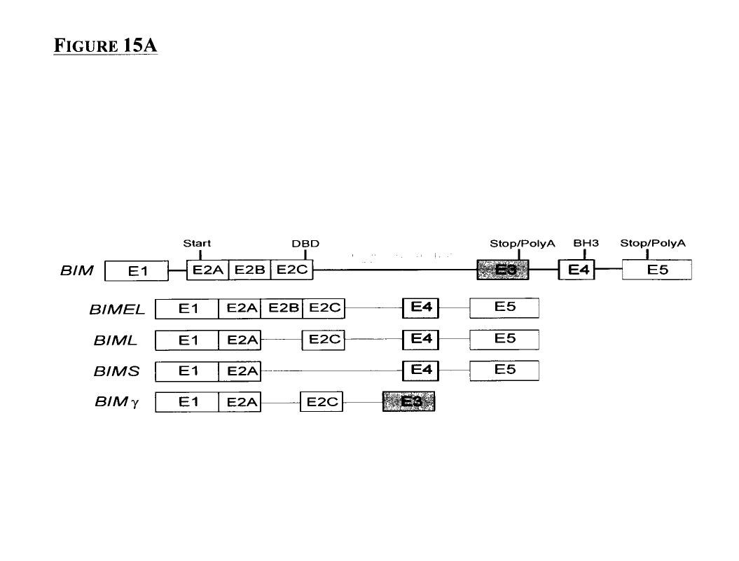

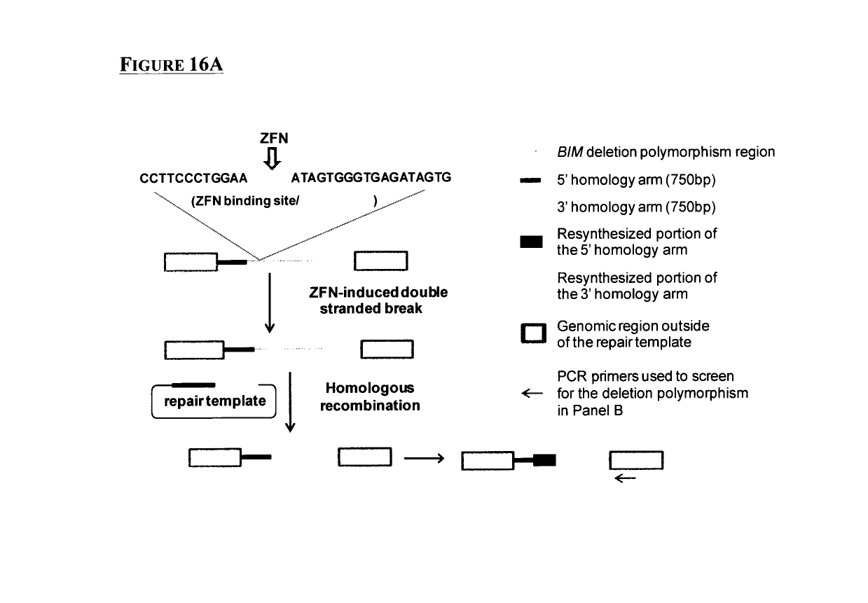

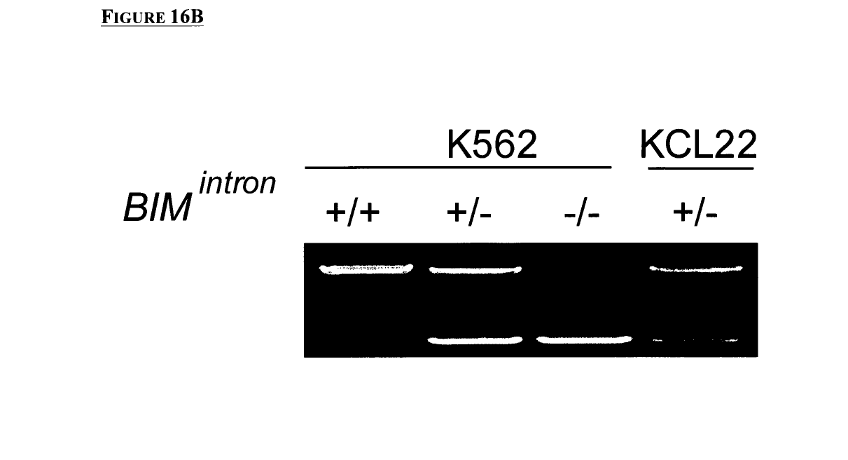

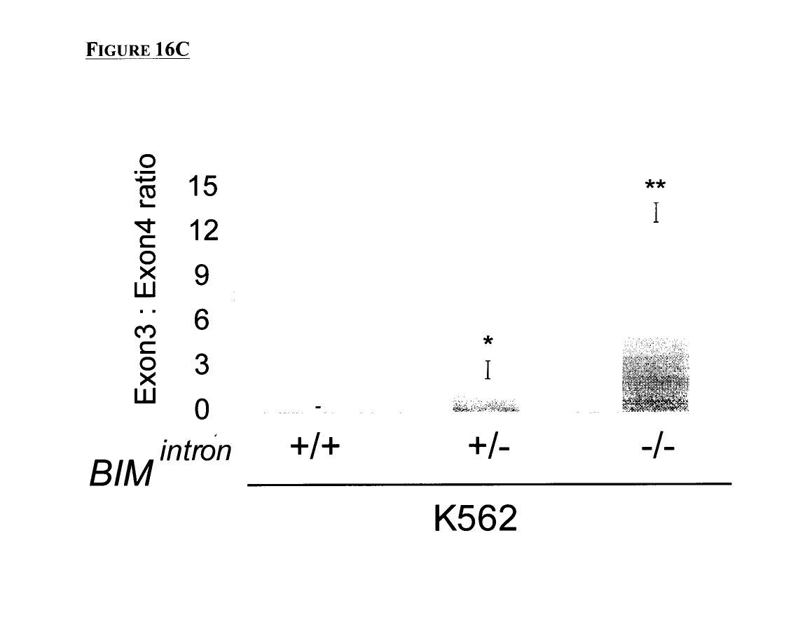

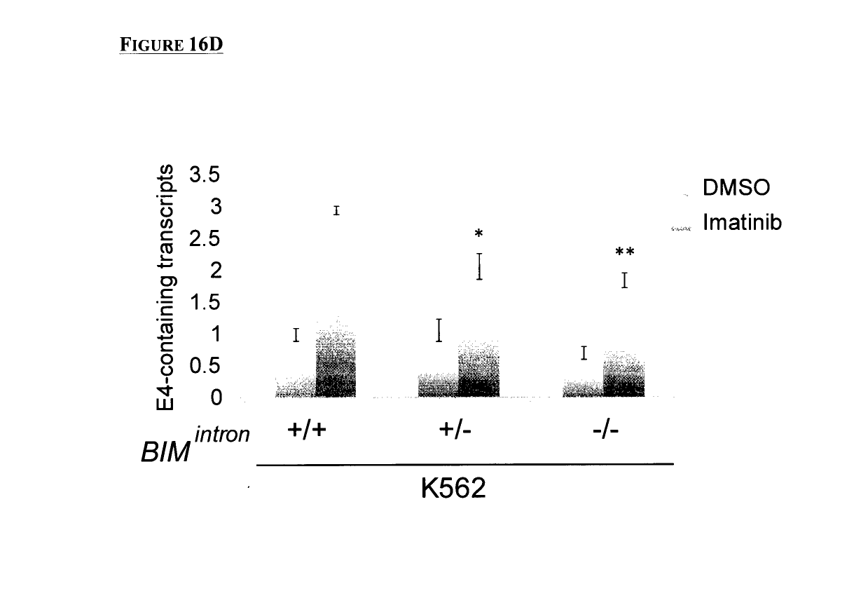

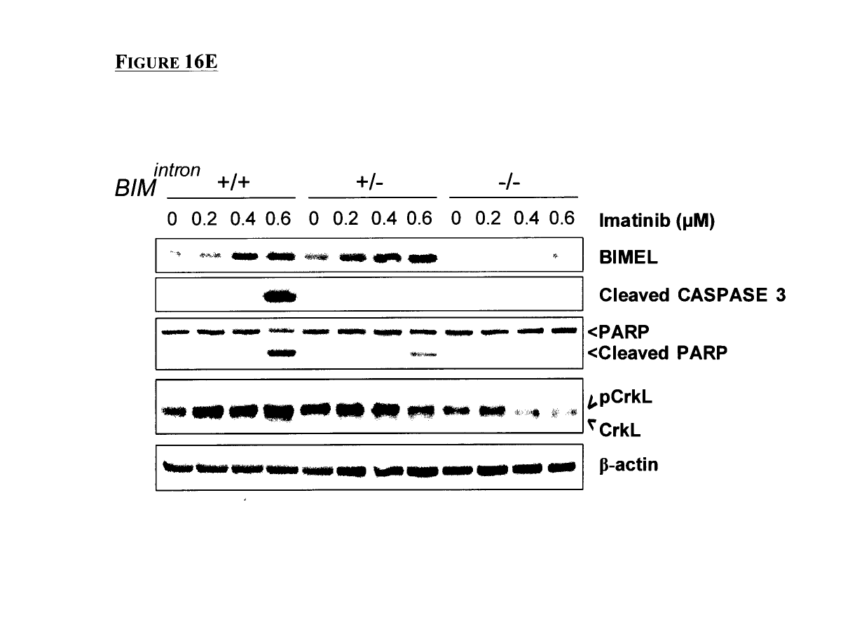

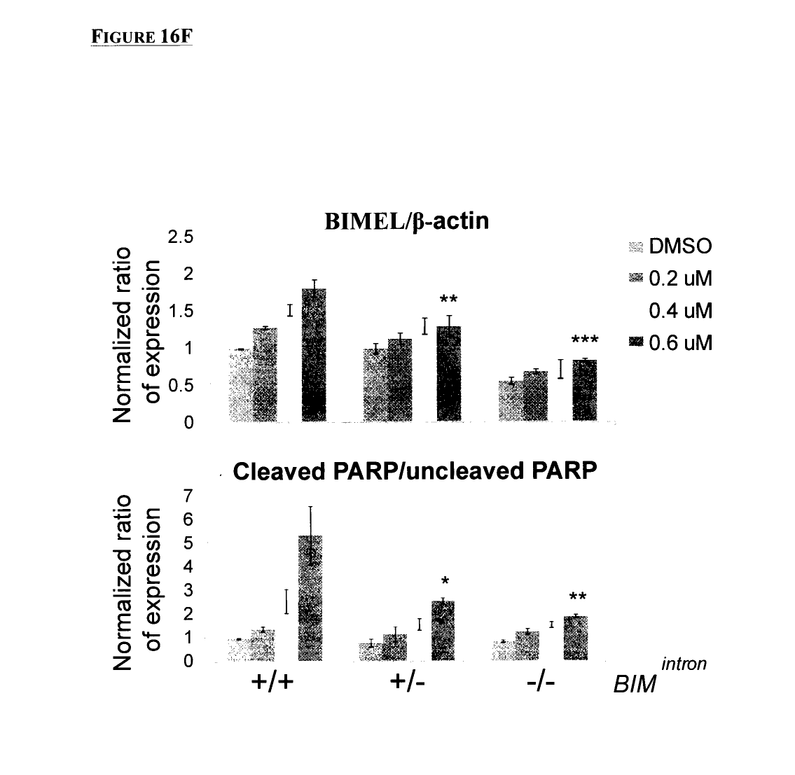

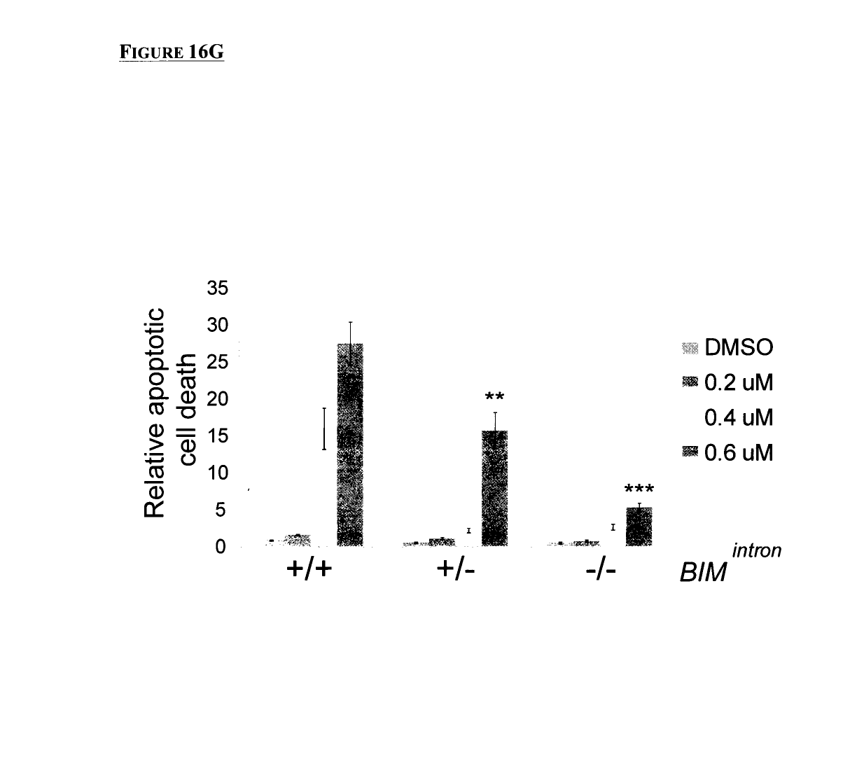

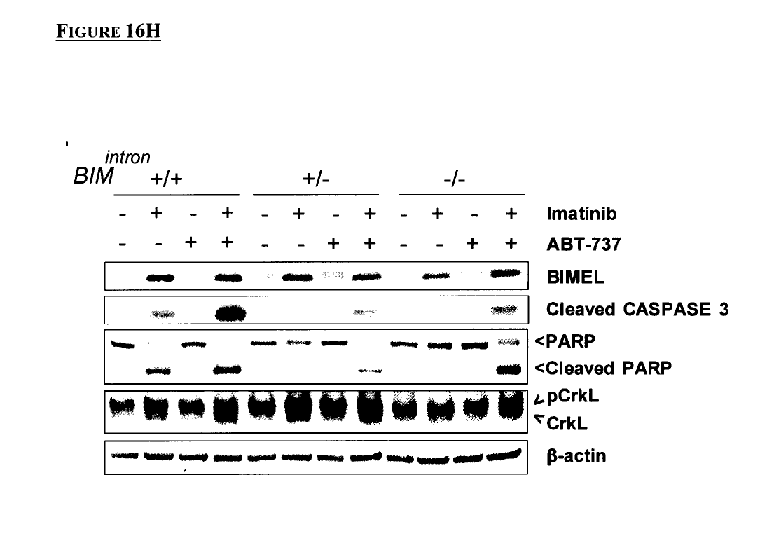

The ZFN was custom-made by Sigma-Aldrich CompoZr™ ZFN Technology (USA). This ZFN cleaved at a site that was 551 bp downstream from the 5′ end of the region that corresponded deletion polymorphism (FIG. 14B). The repair template contained only the 2 flanking homology arms but not the BIM deletion polymorphism region (FIG. 16A). The repair template was constructed by PCR that used the KCL22 genomic DNA, as a template, and the following primers: 5′ CATAAATACCACAGAGGCCCACAGC 3′ (this forward primer is located 619 bp upstream from the 5′ end of the BIM deletion polymorphism region) 5′ CCCTCGAAGACACCTCTATTGGGAGGC 3′ (this reverse primer is located 743 bp downstream of the 3′ end of the BIM deletion polymorphism region).

We isolated a 1362 bp-long PCR product and cloned it into the DNA vector pCR®-Blunt II-TOPO® (Invitrogen, USA). We had sequenced the repair template and confirmed that the correct sequence was obtained. The repair template and ZFN-encoding plasmids were transfected into K562 cells by using the protocol mentioned previously¹⁵. A PCR-based detection assay was developed to detect for the presence of clones that have the BIM deletion polymorphism among the population of transfected K562 cells. The primers used in this PCR-based assay annealed to the BIM intronic region found outside of the repair template (FIG. 16A). As a result, this assay did not detect the repair template (data not shown). The sequences of the primers used were: 5′ GGCCTTCAACCACTATCTCAGTGCAATGG 3′ (this forward primer is located 1507 bp upstream from the 5′ end of the BIM deletion polymorphism region) 5′ GGTTTCAGAGACAGAGCTGGGACTCC 3′ (this reverse primer is located 767 bp downstream of the 3′ end of the BIM deletion polymorphism region).

Genome-edited K562 clones that had the BIM deletion polymorphism were isolated by dilution cloning. The transfected K562 cells were diluted to a density of 2.5 cells/ml. By using a 96-well plate format, each well was then seeded with 200 ul of the diluted transfected K562 cells. Clones that successfully amplified from each well were harvested and the genomic DNA was isolated using a Qiagen DNEasy kit.

Imatinib and ABT-737 Experiments on Genome-Edited K562 Clones

The wildtype and the genome-edited K562 clones were cultured in RPMI-1640 media supplemented with penicillin/streptomycin, glutamine and 20% FBS, and incubated in a humidified incubator at 37° C. with 5% CO₂. In a 6-well plate, 1×10⁶ cells were seeded into each well at a density of 2×10⁵ cells/ml. After incubation with the appropriate drug for 48 hours, the cells were harvested for western analysis (FIG. 16).

For the apoptotic assay, 4×10⁵ cells cells were seeded into each well of a 12-well plate at a density of 2×10⁵ cells/ml. The presence of mono- and oligo-nucleosomes in apoptotic cells was detected by using the Cell Death Detection ELISA (Roche) and following the manufacturer's instructions.

TABLE H1
Table H1. Clinico-Pathalogic Features of the Patients and
CML Cell Line (K562) Used for DNA-PET Analysis

| | | BCR- | Additional | | |
|---|---|---|---|---|---|
| Patient | | ABL | cytogenetic | TKI- | BCR-ABL |
| ID | Phase | Present | abnormalities | resistance | mutation |
| P145¹ | Chronic | Yes | No | No | No |
| P440¹ | Remission | No | No | No | No |
| P308 | Chronic | Yes | No | Yes | No |
| P098 | Blast | Yes | No | Yes | Y253F |
| P022 | Blast | Yes | Yes² | Yes | No |
| K562 | Blast | Yes | Yes | No | No |

¹⁾Same individual at onset (P145), and at remission (P440).
²⁾46, XY, +8, t(9; 22)(q34; q11.2), i(17)(q10)[cp2]/48, idem, +der(22)t(9; 22)[7]/48, idem, +19[cp4]/48, idem, t(12; 17)(p13; q11.2), +19[cp3]/49, idem, +8, +19[2]/46, XY[3].

TABLE H2
Table H2. Statistics of Massively Parallel PET Sequencing on the SOLiD Platform

| Sample | P440 | P145 | P308 | P098 | P022 | K562 |
|---|---|---|---|---|---|---|
| Sequencing | DHH014IHH039 | IHH040 | IHH038 | IHH037 | IHH034 | IHK006007 |
| Library | | | | | | |
| Tags1 | 1,100,736,719 | 406,158,202 | 983,240,880 | 856,013,096 | 1,536,669,332 | 376,077,182 |
| Mappable | 654,761,718 | 187,588,800 | 486,558,654 | 477,870,346 | 854,022,398 | 223,850,946 |
| Tags2 | | | | | | |
| PET3 | 237,888,286 | 57,282,295 | 137,899,606 | 132,002,088 | 256,381,192 | 133,675,193 |
| PET (NR)4 | 60,483,853 | 30,574,510 | 39,548,846 | 53,906,020 | 50,259,818 | 44,065,447 |
| cPET5 span | 6,430-10,460 | 7,339-10,440 | 5,930-8,090 | 7,655-10,272 | 4,410-6,429 | 6,846-10,248 |
| range [bp] | | | | | | |
| cPET (NR)6 | 54,566,588 | 27,920,763 | 31,886,502 | 45,363,132 | 38,068,014 | 40,261,305 |
| Coverage7 | 156.7 | 86.5 | 77.8 | 143.3 | 69.7 | 118.4 |
| dPET (NR)8 | 5,917,265 | 2,653,747 | 7,662,344 | 8,542,888 | 12,191,804 | 3,804,142 |
| dPET | 1,255 | 782 | 1,211 | 1,444 | 1,555 | 1,153 |
| cluster9 | | | | | | |

1Number of 25 bp tags which have been sequenced
2Number of 25 bp tags which have been mapped to the human reference genome (NCBI build 36)
3Number of paired-end tags (PETs) which have been generated after pairing the mapped 25 bp tags
4Non redundant PETs; PETs which have the same starting points for both paired tags have been excluded based on the assumption that they are derived from the same PCR product (not independent biological information)
5Concordant PETs
6Non redundant cPETs (see 4)
7Physical coverage by cPETs; average number of cPET connections crossing a chromosomal position
8Non-redundant discordant PETs (see 4)
9Clusters of dPETs around the same potential rearrangement point which passed “Exclusion of low confidence clusters”

TABLE H3
Table H3. SVs¹⁾ Predicted by DNA-PET in Five
Patient Samples and the K562 Cell Line

| | P440 | P145 | P308 | P098 | P022 | K562 |
|---|---|---|---|---|---|---|
| Deletion | 360 | 350 | 418 | 410 | 467 | 372 |
| Tandem duplication | 11 | 10 | 10 | 9 | 11 | 47 |
| Unpaired inversion | 17 | 17 | 17 | 19 | 21 | 36 |
| Inversion | 46 | 26 | 40 | 42 | 30 | 30 |
| Intra-chr. insertion | 6 | 2 | 2 | 4 | 6 | 2 |
| Inter-chr. insertion | 6 | 2 | 2 | 6 | 6 | 6 |
| Isolated translocation | 14 | 6 | 5 | 16 | 9 | 20 |
| Balanced translocation | 0 | 2 | 2 | 0²⁾ | 2 | 0³⁾ |
| Complex intra-chr. | 34 | 15 | 20 | 18 | 20 | 47 |
| Complex inter-chr. | 9 | 21 | 16 | 7 | 4 | 23 |
| Total | 503 | 451 | 532 | 531 | 576 | 583 |

¹⁾SV statistics are reflected by numbers of dPET clusters (inversions, insertions, and balanced translocations are composed of two dPET clusters per event)
²⁾BCR-ABL translocation but not the reciprocal ABL-BCR is present due to deletion of derivative chromosome 9
³⁾BCR-ABL translocation but not the reciprocal ABL-BCR is present due loss of derivative chromosome 9 or complex rearrangements.

TABLE H4
Table H4. Filtering of Predicted SVs by DNA-PET
in Five Patient Samples and the K562 Cell Line

| | SV1) | Non-redundant2) SV1) |
|---|---|---|
| Raw data | 7,400 | 3,408 |
| After quality filter 3) | 3,176 | 1,349 |
| CML specific4) | 344 | 309 |

1)SV statistics are reflected by numbers of dPET clusters (inversions, insertions, and balanced translocations are composed of two dPET clusters per event)
2)The same SV in different genomes is counted once.
3) dPET clusters were filtered which had a supercluster size >100, or a Blast score >2000, or a Blast alignment type of EC, or a cluster size of 2-5 (see above)
4)After filtering the data by DNA-PET information of 22 normal libraries of 21 normal individuals and published paired-end sequencing data of ten normal samples7, 8.

TABLE H5
Table H5. CML-specific SVs¹⁾ Predicted by DNA-PET
in Five Patient Samples and the K562 Cell Line

| | P440 | P145 | P308 | P098 | P022 | K562 |
|---|---|---|---|---|---|---|
| Deletion | 16 | 17 | 29 | 30 | 49 | 70 |
| Tandem duplication | 4 | 4 | 2 | 3 | 3 | 39 |
| Unpaired inversion | 3 | 2 | 0 | 0 | 2 | 20 |
| Inversion | 0 | 0 | 0 | 2 | 2 | 0 |
| Intra-chr. insertion | 0 | 0 | 0 | 0 | 0 | 0 |
| Inter-chr. insertion | 0 | 0 | 0 | 0 | 0 | 0 |
| Isolated translocation | 1 | 0 | 0 | 4 | 1 | 11 |
| Balanced translocation | 0 | 2 | 2 | 0²⁾ | 2 | 0³⁾ |
| Complex intra-chr. | 3 | 1 | 2 | 1 | 1 | 11 |
| Complex inter-chr. | 0 | 0 | 1 | 0 | 0 | 2 |
| Total | 27⁴⁾ | 26⁴⁾ | 36 | 40 | 60 | 153 |

¹⁾SV statistics are reflected by numbers of dPET clusters (inversions, insertions, and balanced translocations are composed of two dPET clusters per event)
²⁾BCR-ABL translocation but not the reciprocal ABL-BCR is present due to deletion of derivative chromosome 9
³⁾BCR-ABL translocation but not the reciprocal ABL-BCR is present due to loss of derivative chromosome 9 or complex rearrangements.
⁴⁾Four predicted SVs in the chronic phase patient sample P145 were not predicted in remission sample (P440) of the same patient (absence in remission) and seven SVs were predicted in P440 which had no DNA-PET indication in P145 (absence in chronic). In the ‘absence in remission’ category, two of the rearrangement points were BCR-ABL and ABL-BCR and two 5 Kb deletions have been missed in P440 but were identified by PCR in both samples. In the ‘absence in chronic’ category, a deletion and an isolated translocation could not be validated by PCR and have been excluded from the list, the five remaining discrepant SVs could be detected by PCR in both, the chronic and remission sample, and have been missed by DNA-PET in the chronic sample.

TABLE H7

| | Break- | | Break- | | |
|---|---|---|---|---|---|
| Sam- | point | Sequence | point | Sequence | |
| ple | Left | Left¹⁾ | Right | Right | Comment |
| P308 | +chr1: | CTTAATATCATT | +chr1: | GAGTGCAGATA | C-terminal |
| | 120170 | TTTAAAAAGTGG | 120179 | GAAGGACCTGC | truncation of |
| | 138 | ACACATGGGAGG | 262 | TTATGCCCAAC | NBPF7 |
| | | CCAAGGCAGGCA | | CAGCATCCGAT | (Somatic?) |
| | | GATCATGAGGTC | | GACATCAGCCC | |
| | | AAGAGTTCAAGA | | CTTTCTTCCTG | |
| | | CCGCCTGACCAA | | ATCCCCTTTGT | |
| | | CATGGTGAAACC | | CCGGAGCAGAG | |
| | | CTGTCTGTACTA | | TGATGGCTTTT | |
| | | AAAATACAAAAA | | CACACCTCCCA | |
| | | AATTAGCCATGC | | GGGAGGATGTG | |
| | | GAGGTGGTGCAC | | CTCAGCATCAA | |
| | | GCCTG | | C | |
| P308 | +chr1: | GCTCATGCCTAT | +chr1: | CGTGCACCTGT | Duplicates |
| | 200360 | AATCCCAGCACT | 200339 | GATCCCAGCTA | the promoter |
| | 773 | TTGGGAGGCCGA | 079 | CTTGGGAGGCT | and first exon |
| | | GGCAGGTGGATC | | GGGTTGGGAGG | of GPR37L |
| | | ACTTGAGGTCAG | | ATCACCTGAGC | (Somatic?) |
| | | GAGTTCGAGACC | | CCAGGAGGCAG | |
| | | AGCCTGGCCAAC | | AGGTTGCAGTG | |
| | | ATGGTGAAGCCC | | AACCAAGATCA | |
| | | CATCTCTACTAA | | CACCACTAGAC | |
| | | AAATAAAAAATT | | TCCAGCCTGGG | |
| | | AGCCGGGTGTGG | | CGACAGAGTGA | |
| | | TGG | | GACCCTGCCTC | |
| | | | | TTTTTTTT | |
| P098 | +chr2: | ATTTAGATTGTA | +chr2: | CTGTTCTCCAT | Intronic |
| | 111599 | CCTCATGATGAA | 111602 | AGAGGCTGTGC | deletion of |
| | 665 | GGCTAACTCAAC | 569 | CATTTTACATT | BIM |
| | | AAACCCATCAGA | | CCCACCAACAG | (Germline) |
| | | ACAGACACTGGA | | GGCACAAGGGT | |
| | | ACAAAATGACAT | | TCCAGTTTCTC | |
| | | TTCTAAATACCA | | CACATACTTAC | |
| | | TCCAGCTCTGTC | | CAACACTTTTT | |
| | | TTCATAGGCTTC | | TTTTTTTTTTT | |
| | | AGTGAGGTAAAT | | TTAACAGTA | |
| | | CA | | | |
| P308 | +chr2: | ATTTAGATTGTA | +chr2: | ctgttctccat | Intronic |
| | 111599 | CCTCATGATGAA | 111602 | agaggctgtgc | deletion of |
| | 665 | GGCTAACTCAAC | 569 | cattttacatt | BIM |
| | | AAACCCATCAGA | | cccaccaacag | (Germline) |
| | | ACAGACACTGGA | | ggcacaagggt | |
| | | ACAAAATGACAT | | tccagtttctc | |
| | | TTCTAAATACCA | | cacatacttac | |
| | | TCCAGCTCTGTC | | caacacttttt | |
| | | TTCATAGGCTTC | | ttttttttttt | |
| | | AGTGAGGTAAAT | | ttaacagta | |
| | | CA | | | |
| P022 | +chr2: | ATTTAGATTGTA | +chr2: | CTGTTCTCCAT | Intronic |
| | 111599 | CCTCATGATGAA | 111602 | AGAGGCTGTGC | deletion of |
| | 665 | GGCTAACTCAAC | 569 | CATTTTACATT | BIM |
| | | AAACCCATCAGA | | CCCACCAACAG | (Germline) |
| | | ACAGACACTGGA | | GGCACAAGGGT | |
| | | ACAAAATGACAT | | TCCAGTTTCTC | |
| | | TTCTAAATACCA | | CACATACTTAC | |
| | | TCCAGCTCTGTC | | CAACACTTTTT | |
| | | TTCATAGGCTTC | | TTTTTTTTTTT | |
| | | AGTGAGGTAAAT | | TTAACAGTA | |
| | | CA | | | |
| P098 | +chr3: | CTTAACATTTTG | −chr8: | GCATTTCATAA | Together with |
| | 170649 | AAAGCAAAGCCA | 130665 | AGCAGTATGGG | rearrangement |
| | 677 | AATGAAGCTCAA | 028 | TGGCCAGTGCA | point |
| | | TCACGAAAGGTC | | GGGAGCTAGAG | below, 2.7 Mb |
| | | TTTTCTTTCTTT | | CATGAGCTAAT | insertion of |
| | | AAGGTGAGAGAT | | GGCACCAGGGT | chr8 into chr3 |
| | | GGAGCACAAATG | | CCGGTGTAGGC | MDS1 (EVI1) |
| | | AAGCATACCATG | | AGTTAGCTCTG | (Somatic?) |
| | | GTAAATAGGAAC | | CTATATTTGGA | |
| | | TGTGCTCACTTG | | GGGTGTAGATT | |
| | | CAGTCATGCACT | | GTACTCAGCTG | |
| | | ATAGCCATTAAG | | GCAGTGTTTGG | |
| | | CC | | AATCTGTCTTT | |
| | | | | TTGGTCACAAA | |
| P098 | −chr3: | TTATGGCTGGGA | +chr8: | GCTATTGATAT | Together with |
| | 170649 | TGTTTTTGAGAA | 127950 | TTACTTTTTAA | rearrangement |
| | 613 | ATTTATCATATG | 164 | TTTTTTTTTTT | point |
| | | CATTGCTTAGTA | | AGATGAAGTCT | above, 2.7 Mb |
| | | CCTTAAGACCTT | | TGCTCTGTCAC | insertion of |
| | | AAGGCTTAATGG | | CCAGGGCTGGA | chr8 into chr3 |
| | | CTATAGTGCATG | | ATGCAATGGCA | MDS1 (EVI1) |
| | | ACTGCAAGTGAG | | CGATCTCGGCT | (Somatic?) |
| | | CACAGTTCCTAT | | CACTTGCAACC | |
| | | TTACCATGGTAT | | TCCGCCTTCCA | |
| | | GCTTCATT | | GGTTCAAGTGA | |
| | | | | T | |
| P098 | +chr3: | CTGAGATACTTA | +chr3: | ATGGGAAGAAA | Together with |
| | 193420 | AACACGAAAAAC | 193471 | ATAGGGAGATA | rearrangement |
| | 025 | CCCCAGAAATAT | 344 | TAATGGGTGTT | point |
| | | TTTAATATAATA | | GGTGGATGTTA | below, |
| | | GATGTGGTTTGA | | GAATTCCACCA | intronic |
| | | ACCCAGGTATTG | | AAAACACCTCA | insertion in |
| | | GTATGCTTCCTT | | AATTCCATTTA | FGF12 |
| | | TAAGCTCCTCA | | AATTGATTTGA | (Germline) |
| | | | | TATGTAGGTTT | |
| P098 | +chr3: | CCACAAAGGGAA | +chr3: | GTGGTTTTTGG | Together with |
| | 193479 | GTCTCTAAAAAA | 193419 | CCTGGCTGCCT | rearrangement |
| | 385 | CCCTTAACTACA | 901 | AGTGGGATCAa | point |
| | | GTAGGTAAGCGC | | CTGAGATACTT | above, |
| | | TACCCCCAGATT | | AAACACGAAAA | intronic |
| | | CTTACTGAAGTA | | ACCCCCAGAAA | insertion in |
| | | CCAATCAAGTAA | | TATTTTAATAT | FGF12 |
| | | CAATTCTCTGGT | | AATAGATGTGG | (Germline) |
| | | TCCCTTCCTGCT | | TTTGAACCCAG | |
| | | CCTCCCTCTCCA | | GTATTGGT | |
| | | TAGTCTAACTAG | | | |
| | | TCCTGAAGATA | | | |
| P098 | +chr6: | TTTAAAAAAAAG | −chr6: | CCCCCTATTTC | Inversion of |
| | 278995 | AGTGTCTCCTAA | 279074 | GGTTTGGCCCT | HIST1H4J |
| | 79 | AGTGTAAGCGCT | 48 | TTAGATTTCCC | and |
| | | ATCATGCTGGAC | | CTCCCCCACCG | HIST1H4K |
| | | TAATGTTTTAAT | | GGGCGGGACTT | (Somatic?) |
| | | TTAATTTAATTG | | CCCGCCGACTT | |
| | | TTGTCTTGGTAG | | CTTTCAGGTTC | |
| | | AGATGGGGTCTC | | TCAGTTCGGTC | |
| | | GCTATTTTGCTC | | CGCCAACTGTC | |
| | | ACGCAGATCTCA | | GTATAAAGGCG | |
| | | AACTCCTGACGC | | CTGCCTCAGGC | |
| | | CCAGGTACACTC | | CAGAGTCCTCA | |
| | | CCATCTCGGCCT | | CAAAGCGTTGG | |
| | | CCTAAAATGGTG | | GTGAGACTCCT | |
| | | GGATCACAGACG | | CTTGCTCGTCA | |
| | | TCAGCCACCGAG | | TGTCTGGCCG | |
| | | CCCGGTCACTTT | | | |
| | | tTTGTATTcCCC | | | |
| | | ACAGTATTGATG | | | |
| | | TATATCTTCTGC | | | |
| | | GTTCAAAAGCAA | | | |
| | | TTTTTTAAAGCC | | | |
| | | TCATAACGTGGT | | | |
| | | AACAgAATACTt | | | |
| | | TGCACGTTACAA | | | |
| | | AATTCAGAACAC | | | |
| | | GGAAACAAGAAG | | | |
| | | CTCGCTTTTTTT | | | |
| | | Tc | | | |
| P098 | −chr6: | ATTAGTCCTGCC | +chr6: | GTTGATAGAAA | Truncation of |
| | 279002 | TACAGTGACCCC | 279069 | GGGACGCTCAA | HIST1H4J |
| | 00 | GGGTGAATGGCC | 55 | CCACCGAAACC | and |
| | | ACACGTGTGCCG | | GTAGAGGGTGC | HIST1S4K |
| | | CTGTGTTCAAAA | | GGCCCTGGCGC | (Somatic?) |
| | | AACTGTTCCCCC | | TTGAGCGCGTA | |
| | | CAGAACTGACTC | | GACcACATCCA | |
| | | AGAACCAAGCAA | | TGGCGGTGACC | |
| | | AATTCCGTACCT | | GTCTTGCGCTT | |
| | | ACCAAAACCACT | | GGCGTGCTCTG | |
| | | CCTCAGTGTTAC | | TATAGGTCACG | |
| | | CACTCTTCAGCT | | GCGTCCCGGAT | |
| | | GAGAAAGTAGGG | | CACGTTCTCCA | |
| | | TGGCCCTGAAAA | | GGAACAC | |
| | | GGGCCTTTT | | | |
| P022 | −chr7: | AACTTTTGAGAT | −chr12: | ATCATTTAGAT | IMMP2L |
| | 110840 | TGATGAGGGGTT | 106727 | GGTGTGGGTTT | (Somatic?) |
| | 389 | TATTTTGATTCA | 394 | GTATGCAGGAG | |
| | | GTTTTATAATTT | | GGTTGGGAAGT | |
| | | GTGTCTTTTAAT | | AACTAAGGGTG | |
| | | CAATTGGTTCAT | | AGTTAATG | |
| | | TTAATTTATCCA | | | |
| | | AGTTATGGTATA | | | |
| | | AAATTGTTCACA | | | |
| | | ATA“AAAAAAAA | | | |
| | | AAACAAAACACT | | | |
| | | TCTTCGAACAAC | | | |
| | | AACAACAACAAA | | | |
| | | AAA” | | | |
| P098 | +chr9: | ACGATCGCTTGA | +chr22: | TGAATGATGTC | Fusion of |
| | 130893 | GCCCGGGAGTCA | 232497 | TGGAACTTCAA | DOLPP1 and |
| | 272 | AGGCTGCAGCCA | 19 | GGTAGGTCCCC | BUP1/UPB1 |
| | | ACTGTGATCACA | | AGGACCCCTGT | (Somatic?) |
| | | CCACTGCACTCC | | TGTTGCCTGCT | |
| | | AGCCTGAGAAAA | | CCTCTGCTTTG | |
| | | ACCCCATCTCAA | | GCCCTCATCCT | |
| | | AAACAAATATAT | | GCTCTCAGGAA | |
| | | GTGGACAAAGAT | | CTGCTGTTGCG | |
| | | GATCAAATATAA | | GGGTTGCCCAT | |
| | | AAGGAAAAAAGA | | GGCAGCATGAG | |
| | | CACCATGATGG | | GTACCAGCTCC | |
| | | | | CACCTGAGGGC | |
| | | | | TCT | |
| P022 | +chr9: | CCTTAATTTTGA | +chr22: | ACTCTTTGCCC | ABL-BCR |
| | 132596 | CTTAGTCTAATT | 219642 | CATAGTACAGC | (Somatic?) |
| | 651 | TAATCTTTTATG | 93 | GGGGTtTGCTC | |
| | | GTTAGTACTTTT | | TGATTGTAGGG | |
| | | TTTGTGGTCTGT | | GCTTCCCACAT | |
| | | GTAAGAAATCTT | | CCCCCAGGATG | |
| | | TGTGTCTACCCT | | GCTGCCCTtTG | |
| | | AAGGTCACAAAG | | CTGTGGCATCA | |
| | | AAGTTTCA | | CTGTGTAA | |
| P022 | −chr9: | ACATGTATACCT | −chr22: | GGCAAAGGGGA | BCR-ABL |
| | 132596 | GATTTACACAGC | 219642 | CTCCAGGCCAC | (Somatic?) |
| | 651 | AGCAGATGTTCT | 89 | TGTGGGGCACA | |
| | | TTTCAATTAAAC | | GGAAGCAGCTC | |
| | | AGCTAGGTCAAT | | AGTGCTGGAGA | |
| | | ATGAGGGAAAAA | | AGTCGTGGGGT | |
| | | ATGAACCTTCGC | | GCAGGGGGAAG | |
| | | CCCTACACAGAA | | GGAGGGAGGCG | |
| | | AAAACAATTCCA | | AGGCCTGGAGA | |
| | | GGCCAACTATAA | | AGAGTGGGCT | |
| | | ACATGAGATGTA | | | |
| | | AGAGAGTG“G” | | | |
| P308 | +chr9: | CTTTTTTTTTTT | +chr22: | ACACAGCATAC | ABL-BCR |
| | 132673 | TTTGAAATGGAG | 219622 | GCTATGCACAT | (Somatic?) |
| | 558 | TCGTGCTCTGTC | 05 | GTGTCCACACA | |
| | | ACCCAGGCTGGA | | CACCCCACCCA | |
| | | GTGCAGTGGCAC | | CATCCCACATC | |
| | | AATCTCGGCTCA | | ACCCCGACCCC | |
| | | CTGCAACCTCCA | | CTCTGCTGTCC | |
| | | CCTCCCAGGTTC | | TTGGAACCTTA | |
| | | AAGCAATT | | TTACACTTCGA | |
| | | | | GTCACTGGTTT | |
| | | | | GCCTGTATTGT | |
| | | | | G | |
| P308 | −chr9: | CACCTGAGGTCA | −chr22: | GAGCATATGTG | BCR-ABL |
| | 132673 | GGAGTTCGAGAC | 219621 | CAACAGTGAAT | (Somatic?) |
| | 557 | CAGCCTGGCCAA | 96 | GCAAAACAGCT | |
| | | CACGGTGAAACC | | TGACCAAAAGT | |
| | | CCATCTCTACTA | | GCCGGCTGGCA | |
| | | AAAATACAAAAA | | GGTAGCTGGAG | |
| | | TTAGCCAGGCGT | | GAGAGGAGGGC | |
| | | GGTGGTGCATGC | | TGGGAGGACGG | |
| | | CTGTAGTCCCAG | | GAGTGGGGCCA | |
| | | TTACTCGGGAGG | | GATCCAAGGCA | |
| | | CTGAGGCAGAAG | | CAGAGC | |
| | | AA | | | |
| P145 | +chr9: | AAATCTcATAAT | +chr22: | GGCCAAGCCAG | ABL-BCR |
| | 132706 | GTTTTTAAGAAA | 219639 | AAACCGTGGTC | (Somatic?) |
| | 661 | GTTTGCAAATTT | 46 | TGCTCTCCCTC | |
| | | GTTAGGCCACAT | | CGTTAAATGCC | |
| | | | | ATTCTCCATCA | |
| | | | | GTGAGGC | |
| P098 | −chr9: | TGCTGTACTAGA | −chr22: | AACCAGTGACT | BCR-ABL |
| | 132702 | ACTTTTTTTTTT | 219623 | CGAAGTGTAAT | (Somatic?) |
| | 677 | TTTTGAGACTTT | 13 | AAGGTTCCAAG | |
| | | AGAGTCAACATT | | GACAGCAGAGG | |
| | | ATAATTCATTTC | | GGGTCGGGGTG | |
| | | AAGACATGTTGG | | ATGTGGGATGT | |
| | | AATGAGTTTTAG | | GGGTGGGGTGT | |
| | | ATTTTTTTCCCA | | GTGTGGACACA | |
| | | AAACAATAATTA | | TGTGCATAGCG | |
| | | AA | | TATGCTGTGTG | |
| | | | | TGTGACTGAGC | |
| | | | | ATATGTGCAAC | |
| | | | | AGTGA | |
| P145 | −chr9: | GTCAGCCACCGC | −chr22: | GCTGTGGGCGG | BCR-ABL |
| | 132706 | GCCCGGCCTGAA | 219641 | TAAGTTGCAGG | (Somatic?) |
| | 684 | AACCAATTTCTA | 78 | GGGGGGTTTGC | |
| | | GAGCAAGCTTGT | | ATTTTAAAAAA | |
| | | TCAACCCATGGC | | ACTACCTGAAA | |
| | | CCACGGGCTGCA | | CTGGGGATGGA | |
| | | TATGG | | GAGGCCT | |
| K562 | −chr9: | TTCCTGAAATAG | −chr22: | CACAGCGGCCA | BCR-ABL |
| | 132596 | CTTTCTTTCATC | 219627 | GGGTCAAAGGT | (Somatic?) |
| | 963 | AATAAGAATTCT | 32 | GTCCCGTGGTC | |
| | | TTATATTCTATT | | ATCTGAACAAC | |
| | | ATTCATTTCTAG | | CTCCCTGTTTC | |
| | | GATCAAAGAAAC | | CCCACACCCTG | |
| | | CTATAGATTTAG | | CCCGATTAGTC | |
| | | TGCAATCAGAGA | | CTTCCTGGGTG | |
| | | AGAAAATCCTTA | | GAGACCCTGGC | |
| | | AGGGTATTTCTG | | TCGGCCGCTG | |
| | | TTTGGGTATGGA | | | |
| | | AGCTGATAAAAC | | | |
| | | CCACTC | | | |
| P098 | +chr16: | CTCATGCCTGTA | +chr16: | TCAGCACTTTG | Deletion of |
| | 741843 | ATCCTAGCACTC | 742063 | GGAGGCCGAGG | the ADAT1 |
| | 59 | TGGGAGGCCAAG | 72 | TGGGCGAATCA | (Somatic?) |
| | | ATGGGAGGATCG | | CTTGAGGTCAG | |
| | | TTTGAGGCCAGG | | GAGTTCGAGAC | |
| | | AATTAAAGACCA | | CAGCCTGGCCA | |
| | | ATCTGGCCATCA | | ATGTGGTGAAA | |
| | | TAGTGAGACCCC | | CCCTGTTTCTA | |
| | | GTCTCTATTTTT | | CTAAAAATACA | |
| | | TAAAAAACAAAC | | AAAAAATTAGC | |
| | | AAGGGGGCCGGG | | CGGGCGTGGTG | |
| | | CGCAGTGGCTCA | | GCGCATGCCTA | |
| | | CGCCTGTAATC | | TATTCCCACCT | |
| P098 | +chr18: | CCCAGCTCCAGC | +chr18: | CACTGAATGGC | Truncation of |
| | 548456 | TGTCCCTTTTTA | 548539 | ATTCGTGTCTT | promoter & |
| | 10 | CTGTAAAAGTTT | 79 | GAGTCTTCTTT | first exon of |
| | | CGTTTTGAAACA | | GGATCATCTCG | LOC390858 |
| | | ATAGATATGAAA | | GGACACACCAG | (Somatic?) |
| | | ACACTTGGAAAA | | TCAGATGACTG | |
| | | ATGTAAGGCATT | | CATGGCTGTCT | |
| | | ATTACAAACTAA | | TTGGGTGCGGA | |
| | | ATGATTTATACA | | GAGTTGATGAG | |
| | | CTCTCTTTTTCT | | CCTTTACTCTA | |
| | | TTTCTG | | | |
| P022 | +chr19: | AGGCAGAGATAA | +chr19: | CTGTAATCCCA | N-terminal |
| | 401740 | CAGAATAGGAGG | 401806 | GCTATTCAGGA | truncation of |
| | 69 | GCGATGGTTTCA | 87 | AGTTGGGGCAA | GRAMD1A |
| | | ATCCACAGGGCT | | GAGAAT | (Somatic?) |
| | | CTTTGGATGAGA | | | |
| | | AAACATGGGGGT | | | |
| | | GTTGTTAGGTAG | | | |
| | | CCCTGGTCTGCT | | | |
| | | TGATATTTCATT | | | |
| | | TCTGGCCAGGCG | | | |
| | | CAGTGGCACATG | | | |
| | | CC | | | |
| P098 | +chr19: | CTGCTACTGCTA | +chr19: | TTTCACATCCT | IGFL3 |
| | 513145 | TCTCCCATGCCA | 513201 | CCCCTTCCTAA | deletion |
| | 74 | CACCAGCTGCCC | 57 | TACTTGCTTCT | (Somatic?) |
| | | AGAGGTCTGAGA | | TCTATATTCCC | |
| | | ACCTGCCCACAC | | CAAGCACGATG | |
| | | ACAGGGATCATC | | TCTCTGGAAAT | |
| | | GTTCCCACTGCA | | GTCAAGGGGAC | |
| | | AGCTTCTAAGCC | | CgTTTATGAAG | |
| | | AGACATCTGGAG | | CCACTTTATTT | |
| | | GCCCAAGAATCA | | TTTGCTCTGGA | |
| | | GCCCCTAGAACC | | GTCATTAGAGT | |
| | | TACTGACTGG | | TGCCACCTG | |
| P098 | +chr20: | GGAACTCTATAG | +chr20: | GTGGTGCCATC | N-terminal |
| | 482147 | TTTTTAAAATCA | 481936 | TCGGCTCACTG | truncation of |
| | 36 | GAAAATGTCACA | 98 | CAACCTCCACC | TMEM189 |
| | | TTGGTGCAATGC | | TCCCAGGTTCA | (Somatic?) |
| | | TATAATTTAACT | | ATCGATTCTCC | |
| | | TCTTTTCTTTCT | | TGCCTCAGCCT | |
| | | TTTCTTTTTTTT | | CCCGAGTAGCT | |
| | | TTTTTTTTTTTG | | GGGATTATAGG | |
| | | AGATGGAGTTTC | | CACCCGCCACC | |
| | | GCTCTTGTTGCC | | ATGCCCGGCTA | |
| | | CAGGCTGGAGTG | | ATTTTTATATT | |
| | | CA | | TTTTAGTAGAG | |
| | | | | ATGGGGTTTCA | |
| | | | | CCATATTGGCC | |
| | | | | AGGCTGGT | |

Table H7. PCR Validated Rearrangement Points
¹⁾Microhomology of sequences which are the same at both break points are shown in bold and are assigned to the left break point; inserted sequences between the two genomic break points are indicated in bold and underline.

TABLE H8
Table H8. European LeukemiaNet (ELN) Criteria of Overall
Response to First-Line Imatinib in Early Chronic Phase¹⁶

| | | Suboptimal | |
|---|---|---|---|
| | Optimal response | response | Failure |
| Baseline | NA (not | NA | NA |
| | applicable) | | |
| 3 months | CHR and at least | No CyR | Less than CHR |
| | minor CyR | (Ph+ >95%) | |
| | (Ph+ </=65%) | | |
| 6 months | At least PCyR | Less than PCyR | No CyR |
| | (Ph+ </=35%) | (Ph+ >35%) | (Ph+ >95%) |
| 12 months | CCyR | PCyR (Ph+ 1-35%) | Less than PCyR |
| | | | (Ph+ >35%) |
| 18 months | MMR | Less than MMR | Less than CCyR |
| Any time | Stable or | Mutations | Loss of CCyR, |
| | improving MMR | | mutations, CCA |

CHR = complete hematologic response,
PCyR = partial cytogenetic response,
CCyR = complete cytogenetic response,
CCA = clonal chromosomal abnormalities.

TABLE H9
Table H9. Association of the BIM Deletion Polymorphism with Baseline
Characteristics.
Singaporean/Malaysian Cohort

| | BIM | No BIM | |
|---|---|---|---|
| | polymorphism | polymorphism | |
| | (n = 15) | (n = 123) | P value |
| Mean age at diagnosis | 51 | 44 | 0.071* |
| (years) | | | |
| Median time from | 31 | 29 | 0.702\hat{ } |
| diagnosis to start of TKI | | | |
| (days) | | | |
| Gender | | | |
| Male | 10 | 71 | 0.587** |
| Female | 5 | 52 | |
| Race | | | |
| Chinese | 12 | 83 | 0.715** |
| Malay | 3 | 32 | |
| Other East Asian | 0 | 8 | |
| Sokal at diagnosis | | | |
| Low | 3 | 19 | 0.918** |
| Intermediate | 4 | 31 | |
| High | 4 | 39 | |
| Unknown | 4 | 34 | |
| Prior interferon use | | | |
| Yes | 4 | 22 | 0.638** |
| No | 11 | 98 | |
| Unknown | 0 | 3 | |

Japanese Cohort

| | BIM | No BIM | |
|---|---|---|---|
| | polymorphism | polymorphis | |
| | (n = 12) | m (n = 53) | P value |
| Mean age at diagnosis | 62 | 55 | 0.137* |
| (years) | | | |
| Median time from | 4 | 14 | 0.689\hat{ } |
| diagnosis to start of TKI | | | |
| (days) | | | |
| Gender | | | |
| Male | 5 | 32 | 0.335** |
| Female | 7 | 21 | |
| Sokal at diagnosis | | | |
| Low | 4 | 13 | 0.560** |
| Intermediate | 4 | 12 | |
| High | 4 | 5 | |
| Unknown | 0 | 23 | |
| Prior interferon use | | | |
| Yes | 1 | 4 | 1.000** |
| No | 11 | 49 | |

Statistical analysis testing for association between BIM polymorphism and baseline characteristics was carried out using the t-test*, Wilcoxon Rank Sum\hat{ } and Fisher's Exact Test** on the individual cohort tables. Logistic regression analysis on the combined cohort data showed a significant difference in mean age (p = 0.020) between the BIM polymorphism groups (BIM, No BIM) and a non-significant difference between cohorts (p = 0.984). The association between BIM polymorphism and clinical resistance to tyrosine kinase inhibitors was statistically significant (p = 0.022). For Table 1 in the main article, adjustment for age differences was considered appropriate owing to the comparable magnitude of group age differences exhibited in both cohorts (approximately 7 years) and the fact that the age difference in the Singaporean/Malaysian cohort approached statistical significance (p = 0.055).

TABLE H10
Table H10. Clinical Features of Patients with the BIM Deletion Polymorphism.
Imatinib failure

| | | | | | | | | Treatment | | 2nd gen |
|---|---|---|---|---|---|---|---|---|---|---|
| | | Stage at | Sokal | Prior | Initial IM | | BCR-ABL | post IM | | TKI ELN |
| Patient | Race | diagnosis | score | IFN | dose (mg) | IM resistance | mutation | resistance | Present status | response |
| 1 | Japanese | CP | H | N | 400 | No CHR at 18 M | E459K | Bosutinib | PCyR, no MMR | Suboptimal |
| | | | | | | | | | at 21 M | |
| 2 | Japanese | CP | H | N | 400 | No CCyR at 18 M | Nil | Nilotinib | No CyR at 6 M | Failure |
| 3 | Japanese | CP | I | N | 400 | No CCyR at 18 M | Nil | Nilotinib | MMR at 12 M | Optimal |
| 4 | Japanese | CP | H | N | 400 | No CCyR at 18 M | Nil | Nilotinib | CCyR, No MMR | Suboptimal |
| | | | | | | | | | at 12 M | |
| 5 | Japanese | CP | L | N | 400 | No CCyR at 18 M | I418V | Nilotinib | No CHR at 12 M | Failure |
| 6 | Malay | CP | I | Y | 400 | Prog to AP at | Nil | Dasatinib | Not evaluated, | Not |
| | | | | | | 12 M | | | Died | evaluable |
| 7 | Chinese | CP | I | Y | 400 | No CyR at 18 M | Nil | No change | CHR, No CyR | Not |
| | | | | | | | | | | evaluable |
| 8 | Malay | CP | L | N | 400 | No CHR at 12 M | Nil | Dasatinib | CHR, No CyR at | Failure |
| | | | | | | | | | 22 M | |
| 9 | Chinese | CP | I | N | 400 | Loss of CCyR, | Nil | Nilotinib | CHR at 4 M | Not |
| | | | | | | Prog to AP at | | | | evaluable |
| | | | | | | 39 M | | | | |
| 10 | Malay | CP | UNK | N | 400 | No CHR, Prog to | Nil | No treatment | NA, Died | Not |
| | | | | | | AP at 24 M | | | | evaluable |
| 11 | Chinese | CP | I | N | 400 | No CyR, Loss CHR | Nil | Dasatinib | Prog BC at 40 M | Failure |
| | | | | | | at 18 M | | | | |
| 12 | Malay | AP | NA | N | 600 | Loss MMR, Prog | Nil | No treatment | Died | Not |
| | | | | | | BC at 36 M | | | | evaluable |
| 13 | Chinese | CP | UNK | N | 300 | Prog to AP at 15 M | Nil | Dasatinib | Prog to BC at | Failure |
| | | | | | | | | | 9 M, Died | |
| 14 | Chinese | CP | H | N | 400 | No CCyR at 15 M | Nil | No change | No MMR at 48 M | Not |
| | | | | | | | | | | evaluable |
| 15 | Chinese | CP | L | N | 400 | Loss of CCyR at | T277A | Nilotinib | No MMR at 24 M | Suboptimal |
| | | | | | | 12 M | | | | |
| 16 | Chinese | CP | UNK | Y | 600 | No CCyR at 42 M | Nil | Nilotinib | CCyR, No MMR | Suboptimal |
| | | | | | | | | | at 12 M | |
| 17 | Malay | CP | UNK | N | 400 | No CCyR at 36 M | E450A | Dasatinib | MMR at 12 M | Optimal |

Imatinib suboptimal response

| | | | | | | | BCR- | Treatment | | 2nd gen |
|---|---|---|---|---|---|---|---|---|---|---|
| | | Stage at | Sokal | Prior | Initial IM | | ABL | post IM | | TKI ELN |
| Patient | Race | diagnosis | score | IFN | dose (mg) | IM resistance | mutation | resistance | Present status | response |
| 1 | Japanese | CP | H | Y | 400 | CCyR, No MMR | Nil | No change | CCyR no MMR | Not |
| | | | | | | at 18 M | | | | evaluable |
| 2 | Japanese | CP | I | Y | 400 | CCyR, No MMR | Nil | Dasatinib | MMR at 12 M | Optimal |
| | | | | | | at 18 M | | | | |
| 3 | Japanese | CP | L | N | 400 | CCyR, no MMR | Nil | Nilotinib | CCyR, No MMR | Optimal |
| | | | | | | at 18 M | | | at 9 M | |
| 4 | Japanese | CP | H | N | 400 | CCyR, no MMR | Nil | Nilotinib | MMR at 12 M | Optimal |
| | | | | | | at 18 M | | | | |
| 5 | Japanese | CP | I | N | 400 | CCyR, no MMR | Nil | Nilotinib | MMR at 12 M | Optimal |
| | | | | | | at 18 M | | | | |
| 6 | Japanese | CP | L | N | 400 | CCyR, no MMR | Nil | Nilotinib | MMR at 12 M | Optimal |
| | | | | | | at 18 M | | | | |
| 7 | Japanese | CP | I | N | 400 | CCyR, no MMR | Nil | Nilotinib | No MMR at 3 M | Not |
| | | | | | | at 18 M | | | | evaluable |
| 8 | Chinese | CP | I | N | 400 | no MMR at 18 M | Nil | Imatinib | No MMR | Suboptimal |
| | | | | | | | | 600 | | |
| | | | | | | | | mg QD | | |
| 9 | Chinese | CP | I | N | 400 | no MMR at 18 M | Nil | Imatinib | MMR at 24 M | Optimal |
| | | | | | | | | 600 | | |
| | | | | | | | | mg QD | | |

Imatinib optimal response

| | | Stage at | Sokal | Prior | Initial IM | Present |
|---|---|---|---|---|---|---|
| Patient | Race | diagnosis | score | IFN | dose (mg) | status |
| 1 | Japanese | CP | H | N | 400 | MMR |
| 2 | Japanese | CP | L | Y | 400 | MMR |
| 3 | Japanese | CP | I | N | 400 | MMR |
| 4 | Japanese | CP | L | N | 400 | MMR |
| 5 | Chinese | CP | H | N | 600 | MMR |
| 6 | Chinese | CP | H | N | 400 | MMR |
| 7 | Chinese | CP | L | N | 400 | MMR |
| 8 | Chinese | CP | L | N | 400 | MMR |
| 9 | Chinese | CP | I | Y | 400 | CMR |
| 10 | Chinese | CP | UNK | N | 400 | MMR |
| 11 | Chinese | CP | H | Y | 400 | MMR |

Abbreviations: IFN, interferon; IM, imatinib; TKI, tyrosine kinase inhibitor; ELN, European Leukemia Net; CP, chronic phase; AP, accelerated phase; BC, blast crisis; H, high; L, low; UNK, unknown; CHR, complete hematologic response; PCyR, partial cytogenic response; CCyR, complete cytogenetic response; MMR, major molecular response; CMR, complete molecular response.

TABLE H11
Table H11. Association of the BIM Deletion
Polymorphism with Imatinib Resistance in the
Absence of a BCR-ABL Kinase Domain Mutation.

| | | No BIM |
|---|---|---|
| | BIM Deletion | Deletion |
| | polymorphism | polymorphism |
| | No. (%) | No. (%) |
| Singaporean/Malaysian | | |
| Cohort (n = 138) | | |
| Group I (Resistant with no | 8 (53) | 34 (28) |
| BCR-ABL mutation) | | |
| Group II (Resistant with | 2 (13) | 25 (20) |
| BCR-ABL mutation) | | |
| Group III (Sensitive) | 5 (33) | 64 (52) |
| Japanese Cohort | | |
| (n = 65) | | |
| Group I (Resistant with no | 8 (67) | 25 (47) |
| BCR-ABL mutation) | | |
| Group II (Resistant with | 2 (17) | 5 (9) |
| BCR-ABL mutation) | | |
| Group III (Sensitive) | 2 (17) | 23 (43) |

The two cohorts of patients in Table 1 were each further divided into three groups: resistant without a BCR-ABL mutation (Group I), resistant with a BCR-ABL mutation (Group II), and sensitive (Group III). Accordingly, these data were analyzed using logistic regression. There was no significant difference between Singaporean/Malaysian and Japanese cohorts (p = 0.31), but significant differences among Groups as indicated by the Fisher's exact test (p = 0.03). Pairwise 95% confidence intervals on odds ratios demonstrated significant differences between Group I vs Group III, and Group I vs. Group II and III, but not between Group I vs Group II (odds ratio = 2.03, 95% CI of 0.68-7.58), and Group II vs. Group III (odds ratio = 1.66, 95% CI of 0.41-5.89). Comparing Group I vs. Group III gave an odds ratio of 3.37 (95% CI 1.35-9.23), and Group I vs. Groups II and III gave and an odds ratio of 1.90 (95% CI of 2.08-4.35).
All calculations were performed using SAS V9.2 17.

TABLE H12
Table H12. Thresholds Defined by the
‘Exclusion of Low Confidence Clusters’

| | | Stretched | | Proximity |
|---|---|---|---|---|
| | Sequencing | dPET | Chimeric dPET | threshold |
| Sample | Library | threshold¹⁾ | threshold²⁾ | (in Mbp)³⁾ |
| P440 | DHH014IHH039 | 6 | 3 | 3.4 |
| P145 | IHH040 | 6 | 3 | 1.6 |
| P308 | IHH038 | 8 | 3 | 2.4 |
| P098 | IHH037 | 9 | 3 | 3.2 |
| P022 | IHH034 | 8 | 3 | 3.0 |
| K562 | IHK006007 | 6 | 3 | 2.4 |

¹⁾Minimum dPET cluster size for (deletion) clusters where the left anchor start is less than 2X the maximum library size away of the right anchor start
²⁾Minimum dPET cluster size for all dPET clusters except those which are ‘stretched up to 2X the library size’ and within the ‘proximity threshold’
³⁾dPET clusters of size 2 are allowed if both anchors are within the indicated distance (except ‘stretched dPET’ rule)

The dPET clusters which passed the above mentioned thresholds were the basis for the superclustering (see below).

TABLE H13
Table H13. IC50 of KCL22, KYO-1, and NCO2 Cell Lines
Treated With Imatinib as Determined by Annexin V Staining.

| | IC [symbol] (μM) |
|---|---|
| KCL22 | >10 μM |
| KYO-1 | 0.27 μM |
| NCO2 | 0.34 μM |

[symbol]

indicates data missing or illegible when filed

### Example 112

### References for Example H1

The following are the references for Example H1. Materials and Methods
1. Hillmer, A. M., et al. Comprehensive long-span paired-end-tag mapping reveals characteristic patterns of structural variations in epithelial cancer genomes. Genome Res (2011).
2. McDermott, U., Downing, J. R. & Stratton, M. R. Genomics and the continuum of cancer care. N Engl J Med 364, 340-350 (2011).
3. Calabretta, B. & Perrotti, D. The biology of CML blast crisis. Blood 103, 4010-4022 (2004).
4. Foong, A. W., et al. Rationale and methodology for a population-based study of eye diseases in Malay people: The Singapore Malay eye study (SiMES). Ophthalmic Epidemiol 14, 25-35 (2007).
5. Lavanya, R., et al. Methodology of the Singapore Indian Chinese Cohort (SICC) eye study: quantifying ethnic variations in the epidemiology of eye diseases in Asians. Ophthalmic Epidemiol 16, 325-336 (2009).
6. Altschul, S. F., Gish, W., Miller, W., Myers, E. W. & Lipman, D. J. Basic local alignment search tool. J. Mol Biol 215, 403-410 (1990).
7. Kidd, J. M., et al. Mapping and sequencing of structural variation from eight human genomes. Nature 453, 56-64 (2008).
8. Korbel, J. O., et al. Paired-end mapping reveals extensive structural variation in the human genome. Science 318, 420-426 (2007).
9. Rhead, B., et al. The UCSC Genome Browser database: update 2010. Nucleic Acids Res 38, D613-619 (2010).
10. Barnes, D. J., et al. Bcr-Abl expression levels determine the rate of development of resistance to imatinib mesylate in chronic myeloid leukemia. Cancer Res 65, 8912-8919 (2005).
11. Quintas-Cardama, A. & Cortes, J. Molecular biology of bcr-abl1-positive chronic myeloid leukemia. Blood 113, 1619-1630 (2009).
12. Sinclair, P. B., et al. Large deletions at the t(9;22) breakpoint are common and may identify a poor-prognosis subgroup of patients with chronic myeloid leukemia. Blood 95, 738-743 (2000).
13. Frazer, K. A., et al. A second generation human haplotype map of over 3.1 million SNPs. Nature 449, 851-861 (2007).
14. Korn, J. M., et al. Integrated genotype calling and association analysis of SNPs, common copy number polymorphisms and rare CNVs. Nat Genet. 40, 1253-1260 (2008).
15. Urnov, F. D., et al. Highly efficient endogenous human gene correction using designed zinc-finger nucleases. Nature 435, 646-651 (2005).
16. Baccarani, M., et al. Chronic myeloid leukemia: an update of concepts and management recommendations of European LeukemiaNet. J Clin Oncol 27, 6041-6051 (2009).
17. Thomas, D. G. Algorithm AS-36. Exact Confidence Limits for the Odds Ratio in a 2×2 Table. Applied Statistics 20, 105-110 (1971).
18. Krzywinski, M., et al. Circos: an information aesthetic for comparative genomics. Genome Res 19, 1639-1645 (2009).

### Example H3

### Introduction

Most patients with chronic myeloid leukemia (CML) respond to therapy with the BCR-ABL tyrosine kinase inhibitor (TKI)¹. However, 15-20% of patients do not respond optimally to TKIs², and have TKI resistance. There are currently no reliable biomarkers for predicting clinical resistance or sensitivity to alternative therapies. Here we identify a novel mechanism of intrinsic TKI resistance mediated by a 2.9 Kb deletion polymorphism in the BIM gene. The deletion biased splicing away from BIM isoforms containing the pro-death BH3 domain, and impaired the ability of TKIs to upregulate BH3-containing transcripts. This led to a blunted apoptotic response and intrinsic TKI resistance, a resistance that could be overcome with BH3-mimetic drugs. Interestingly, the polymorphism was restricted to East-Asian individuals (12.3% carrier rate), and was absent in Africans and Caucasian (0%). East-Asian CML patients (n=203) exhibited a strong association between the presence of the polymorphism and inferior TKI responses (p=0.02). Our results illustrate how a common deletion polymorphism can influence clinical responses to TKIs, a class of drugs with proven efficacy against a wide range of kinase-driven cancers. Importantly, by elucidating the effects of the polymorphism on BIM function, we demonstrate that BH3-mimetics can overcome this form of TKI resistance.

BIM is a family member of the BH3-only proteins that activate cell death by either opposing the prosurvival members of the BCL2 family (BCL2, BCL-XL, MCL1, and A1) or binding directly to the distal effectors of cell death, BAX and BAK³. BCR-ABL1 maintains a survival advantage for CML cells, in part, by suppressing BIM transcription, as well as targeting BIM protein for proteasomal degradation via ERK-dependent phosphorylation⁴⁻⁶. Importantly, TKIs induce apoptosis in CML cells via upregulating BIM transcription, while preventing BIM expression is sufficient to confer in vitro TKI resistance⁴⁻⁶. Clinical TKI resistance is most commonly associated with acquired somatic mutations in the kinase domain of BCR-ABL 1 that decrease the ability of TKIs to bind and inhibit BCR-ABL1⁷. However, such mutations cannot be found in the majority of patients who either fail to respond altogether, or have suboptimal TKI responses, as defined by clinical criteria². Further, the variability of patient responses to TKIs raises the possibility that germline variants or polymorphisms also contribute to drug resistance in CML^{2,8}.

### Example H4

### Structural Variant in Intron 2 of BIM Gene in TKI Sensitive CML

To identify novel TKI resistance mechanisms in CML, we used massively parallel DNA sequencing of paired-end ditags^{9,10} (FIG. 14) to interrogate the genome of five CML samples obtained from patients who were sensitive or resistant to TKIs (Table H1).

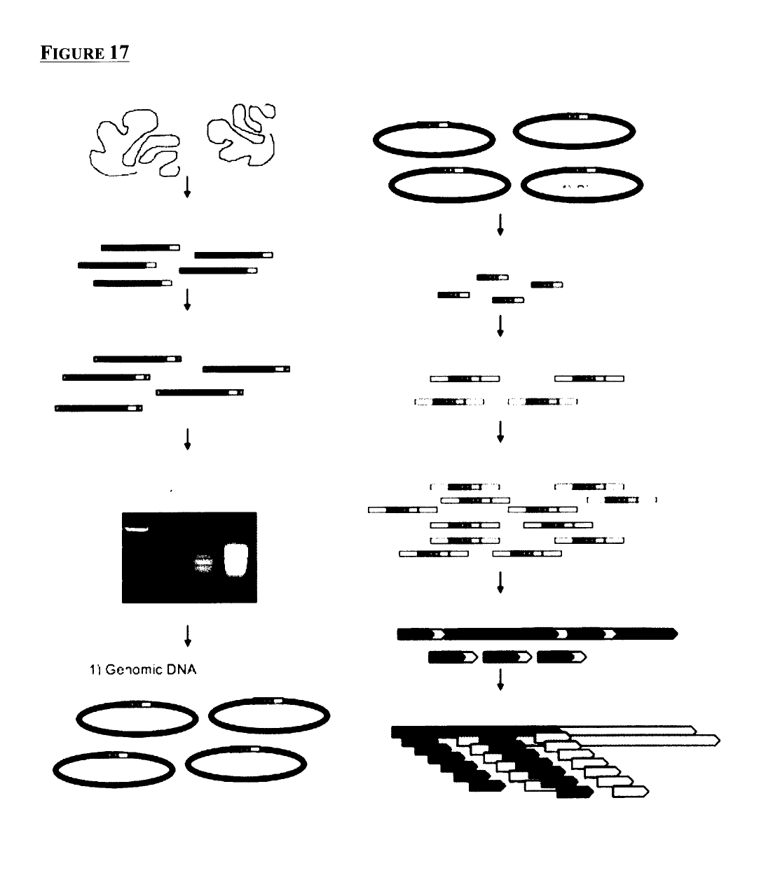

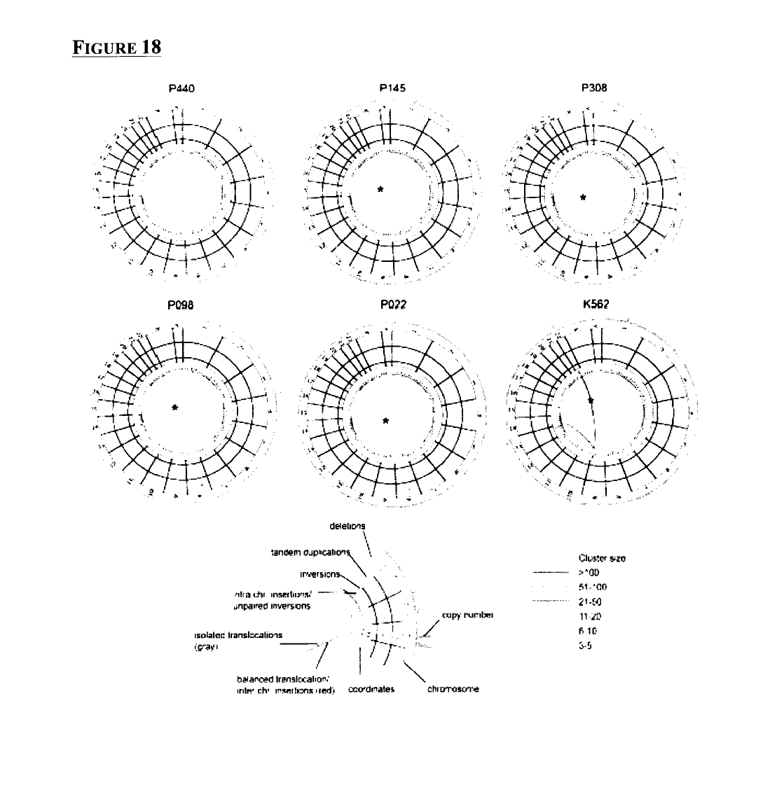

We identified the BCR-ABL1 translocation in all CML samples, but not controls, as well as several novel structural variations (SV) (FIG. 18 and FIG. 19, and Tables H2 to H7).

Among SVs that were common to all resistant samples, one in particular attracted our attention since it occurred in intron 2 of the BIM gene (FIG. 14A). This SV comprised an identical 2,903 bp deletion (FIG. 14A, FIG. 14B and FIG. 14C), suggesting it was germline and polymorphic. Indeed, upon screening 2465 normal individuals, we found the deletion polymorphism to occur commonly in East-Asian individuals (12.3-14.3% carrier frequency), but was absent in African and European populations (0%) (FIG. 14D).

### Example 115

### Splicing of BIM Gene

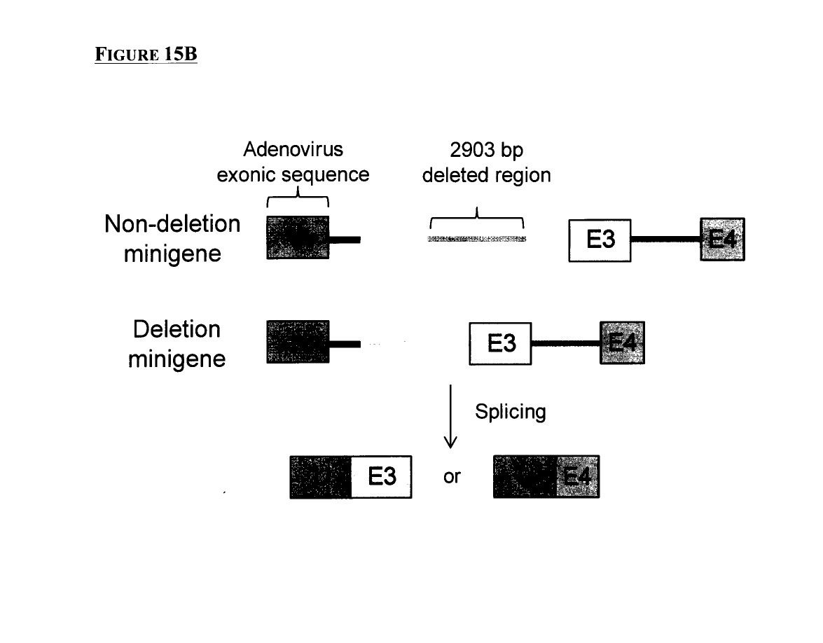

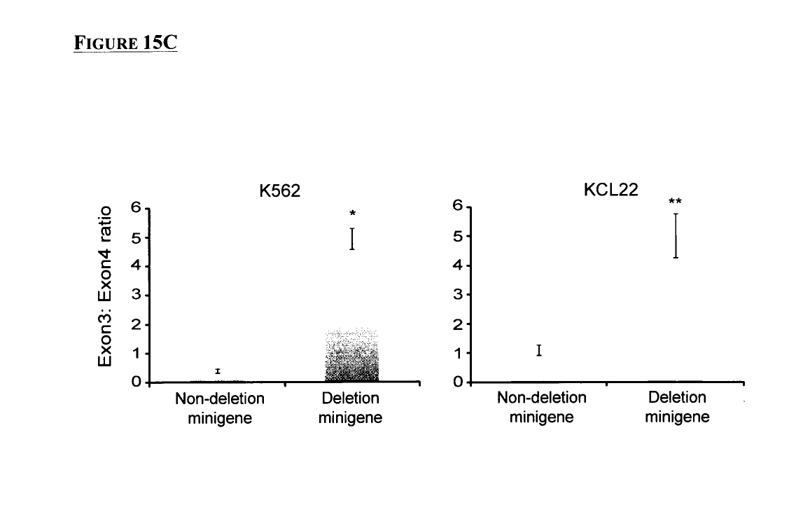

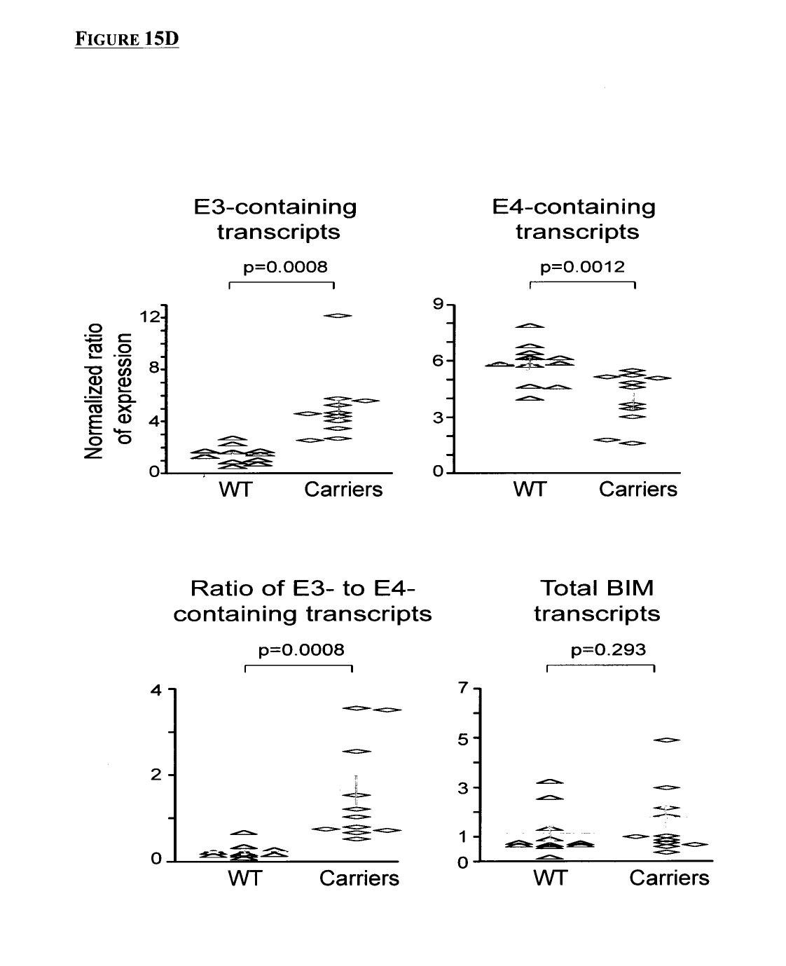

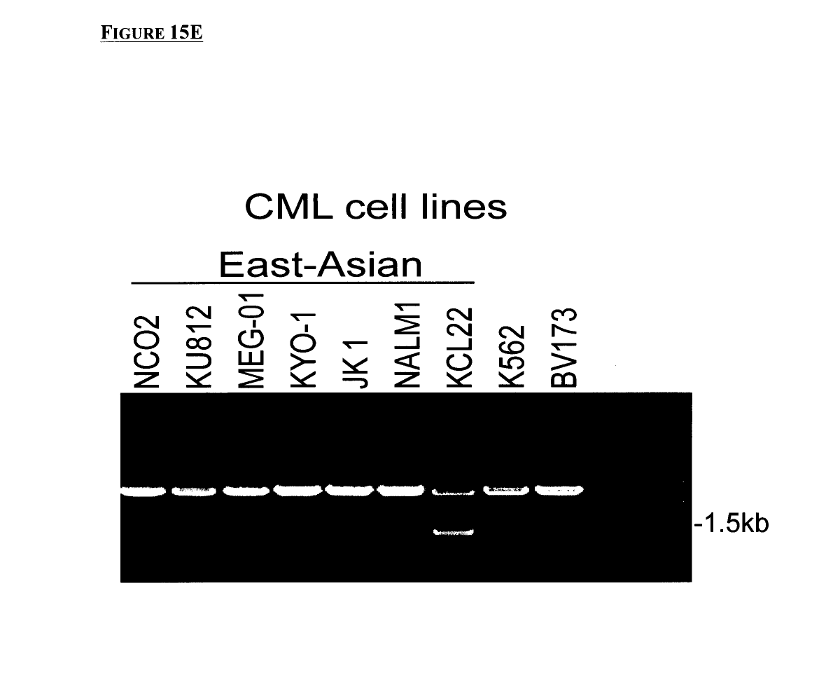

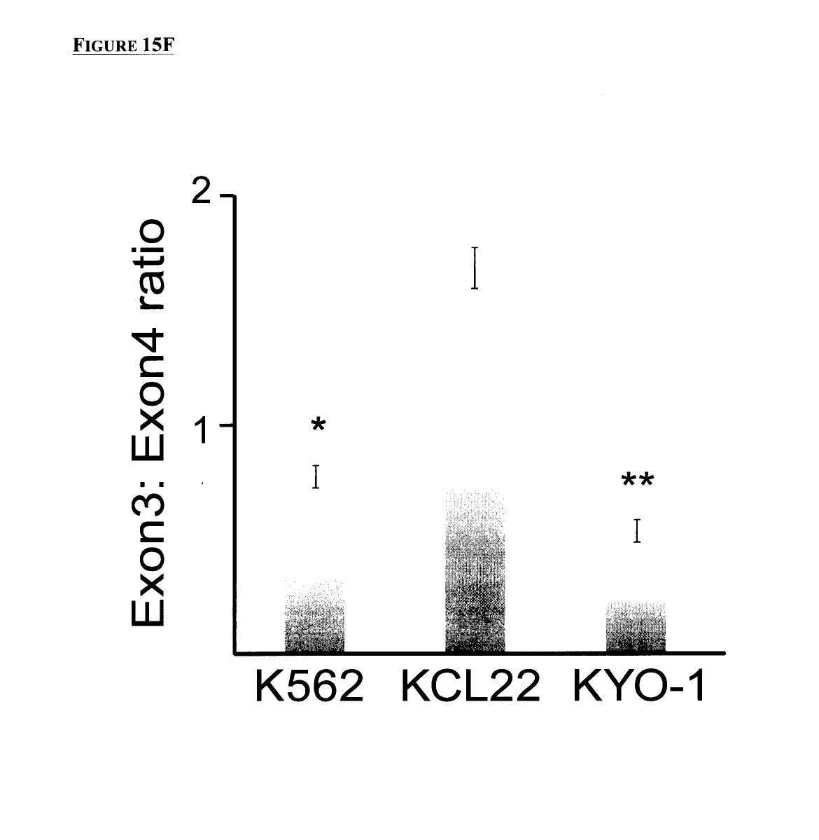

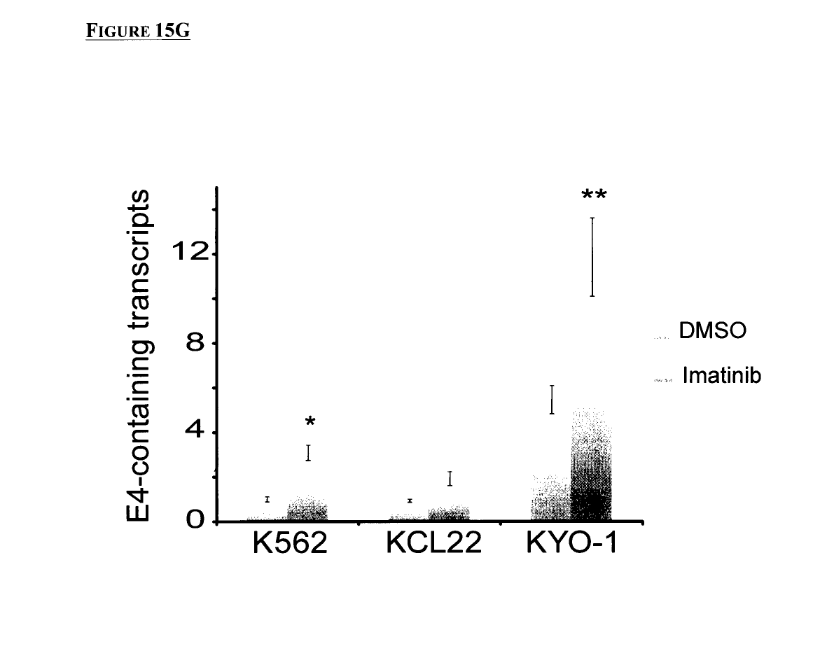

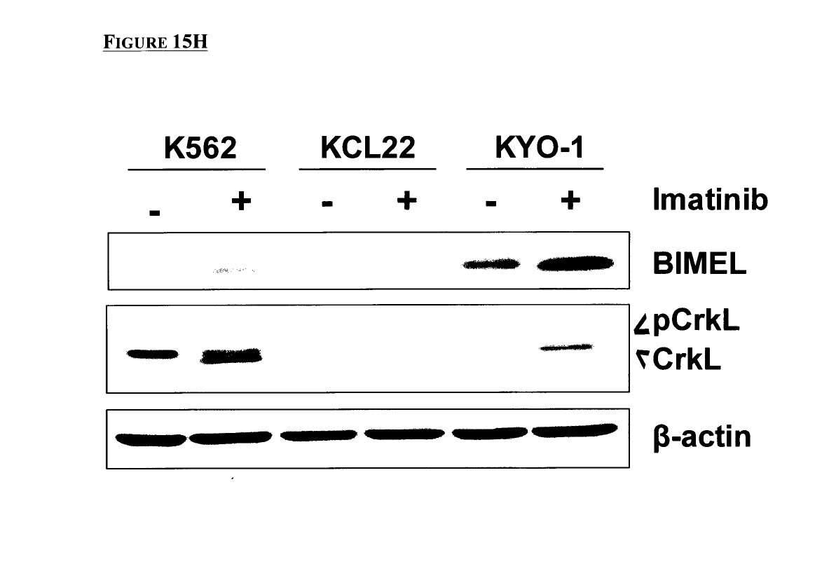

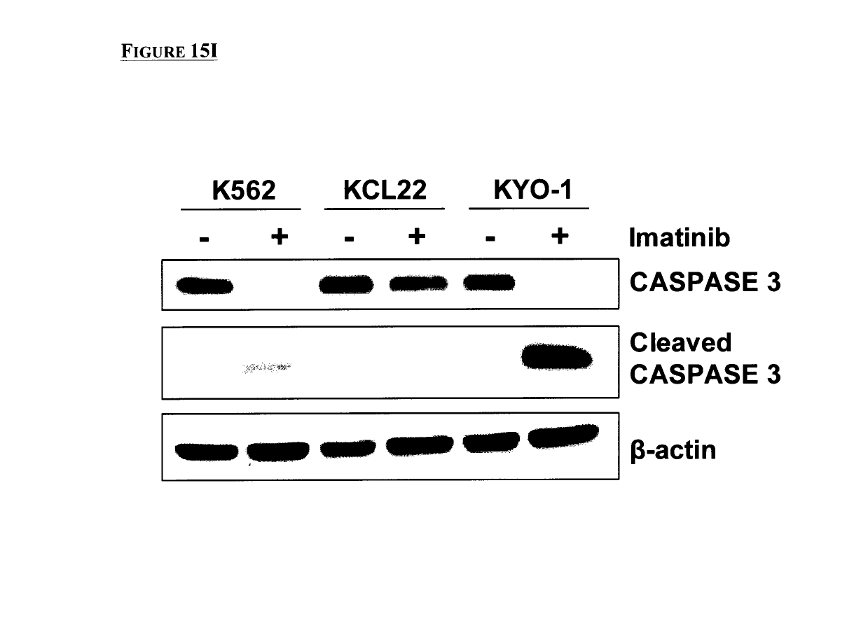

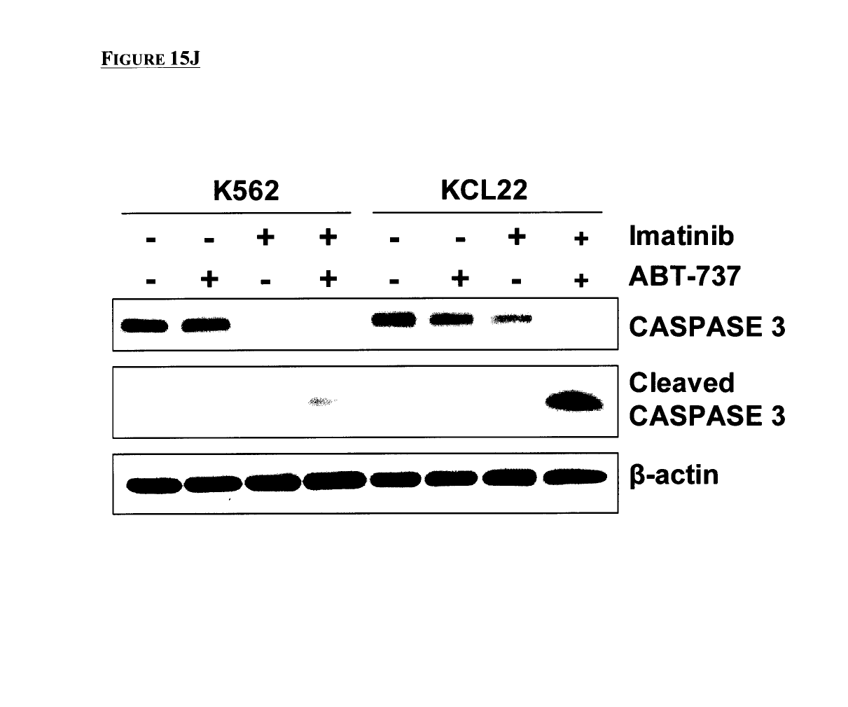

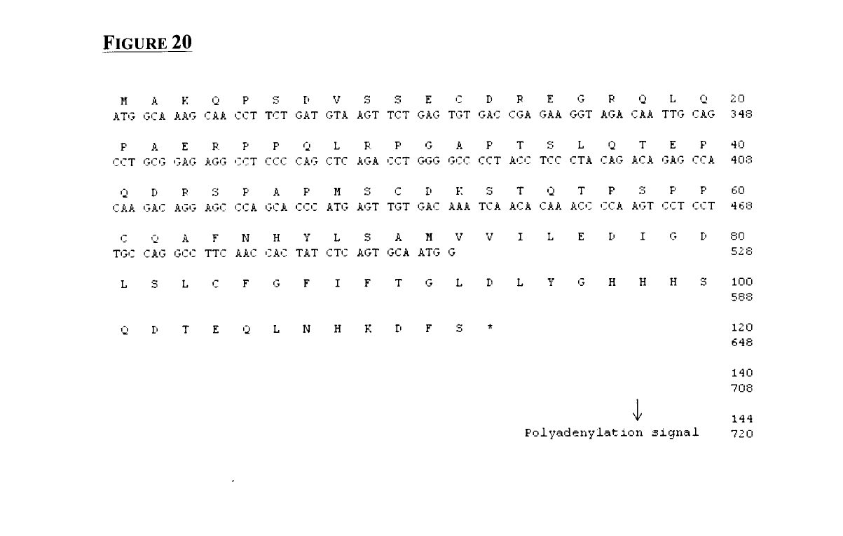

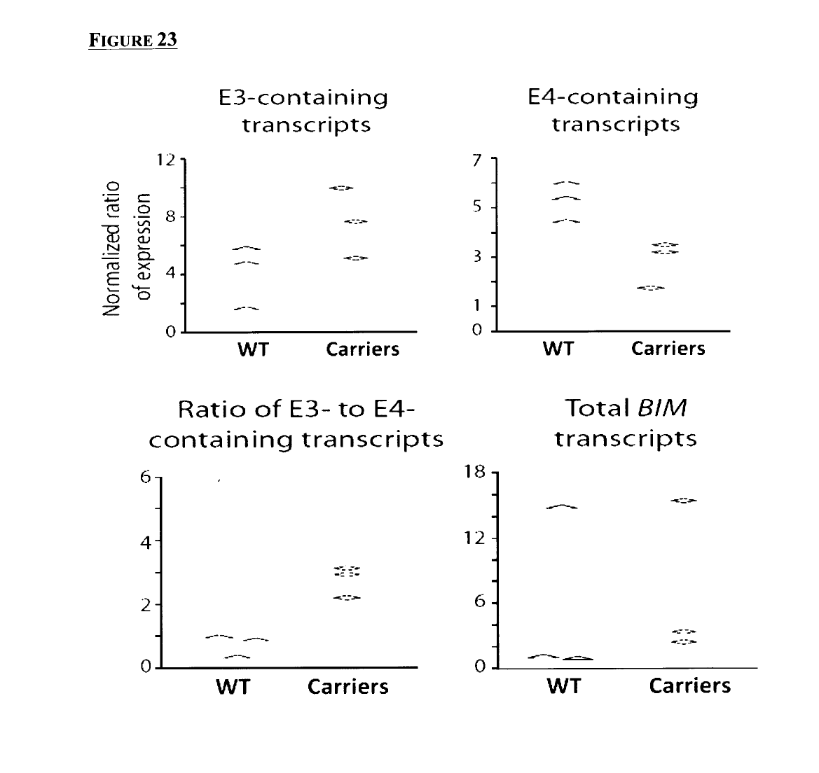

Inspection of BIM gene structure suggested that the deletion polymorphism would result in splicing of exon 3 and 4 in a mutually exclusive manner, a possibility suggested by its proximity (107 bp) to the 5′ end of the intron-exon boundary of exon 3, as well as the presence of a stop codon and polyadenylation signal within exon 3 itself (FIG. 15A and FIG. 20)¹¹. To confirm the effect of the polymorphism on BIM expression, we constructed a minigene to measure exon 3 vs exon 4 splicing in the presence or absence of the deletion (FIG. 15B)¹², and found the deletion favored splicing to exon 3 over exon 4 bp at least 5-fold (FIG. 15C). Importantly, primary CML cells from patients exhibited the same phenomenon, since polymorphism-containing samples expressed higher levels of exon 3-vs exon 4-containing transcripts, while general BIM transcription was unaffected (FIG. 15D). Similar results were observed in lymphoblastoid cell lines obtained from normal HapMap individuals, indicating a cell lineage-independent effect (FIG. 23). Because the pro-apoptotic BH3 domain is found exclusively in exon 4 (FIG. 14A), and is required for BIM apoptotic function^{13,14}, our observations suggested a novel mechanism for TKI resistance. In this model, upon TKI exposure, polymorphism-containing CML cells would favor expression of exon 3- vs exon 4-containing BIM transcripts, decreased expression of BH3-containing BIM isoforms, and consequently impaired BH3 domain-dependent apoptosis. To facilitate these studies, we identified a Japanese CML cell line, KCL22¹⁵, that contained the deletion (FIG. 15E), and confirmed they expressed an increased exon 3/exon 4 transcript ratio compared to non-deletion containing cells (FIG. 15F). KCL22 cells also demonstrated decreased induction of exon 4-containing transcripts following TKI exposure (FIG. 15G), as well as lower levels of BIMEL protein, a major BH3-containing BIM isoform (FIG. 15H)¹⁶. Consistent with previous reports^{15,17,18}, KCL22 cells were resistant to imatinib, despite effective BCR-ABL inhibition (FIG. 15H and Table H13), and demonstrated impaired apoptotic signaling upon imatinib exposure (FIG. 15I).

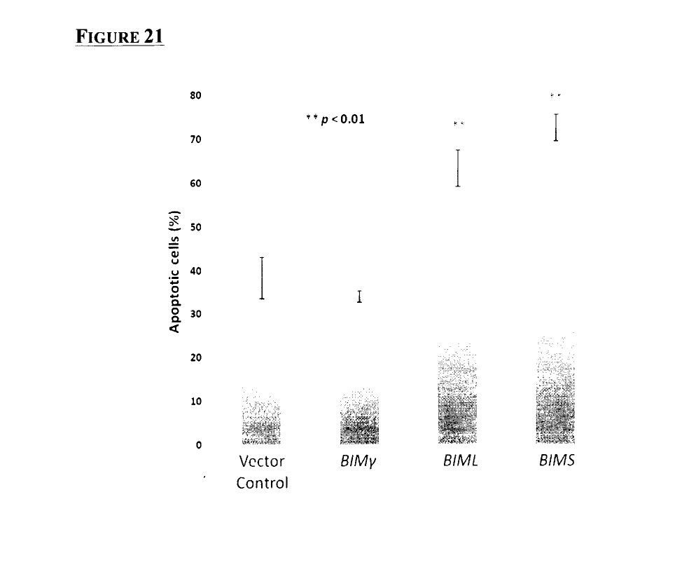

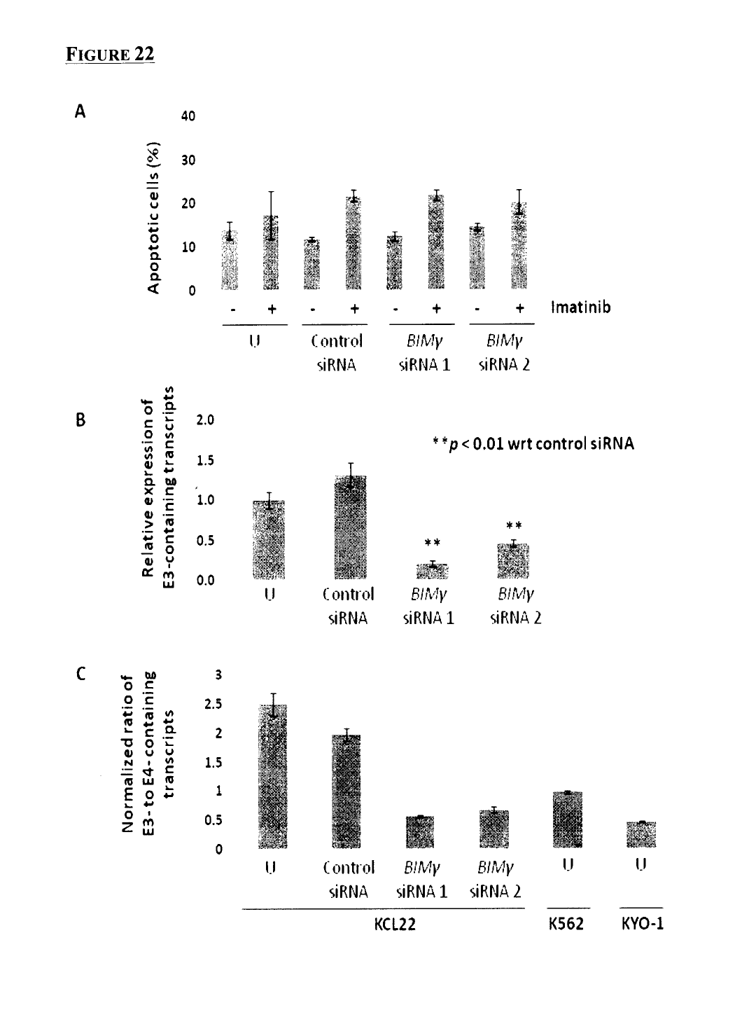

KCL22 cells were also exquisitely sensitive to increased expression of exon 4/BH3-containing (but not exon 3-containing) BIM isoforms (FIG. 21), while siRNA-mediated knockdown of exon 3-containing transcripts did not sensitize KCL22 cells to imatinib (FIG. 22), suggesting that the impaired imatinib-induced apoptosis could be restored by the addition of BH3 mimetic drugs¹⁹. As shown in FIG. 14J, we found that this was indeed the case.

### Example H6

### Gene Targeting to Recreate Deletion Polymorphism

We next employed zinc finger nuclease-facilitated gene targeting to precisely recreate the deletion polymorphism in the BIM gene of imatinib-sensitive K562 CML cells (FIG. 15A). We then analyzed these cells for changes in BIM splicing and expression, as well as TKI-induced apoptosis. We generated subclones that were heterozygous (K562-BIM^{intron+/-}) or homozygous (K562-BIM^{intron-/-}) for the deletion polymorphism (FIG. 16B), and confirmed an increased exon 3/exon 4 transcript ratio in these cells in a polymorphism-dosage-dependent manner (FIG. 16C). Deletion polymorphism-containing cells also exhibited decreased induction of exon 4-containing transcripts following imatinib exposure (FIG. 16D), impaired upregulation of BIMEL protein, and both diminished apoptotic signaling and cell death (FIG. 16E, FIG. 16F and FIG. 16G). As in KCL22 cells, the addition of ABT-737 to imatinib enhanced the ability of the latter to activate apoptosis in polymorphism-containing cells (FIG. 16H). Taken together, our studies establish that the polymorphism impairs the apoptotic response to imatinib by biasing splicing away from BH3-containing BIM isoforms, and is sufficient to render CML cells intrinsically resistant to imatinib. Importantly, we also show that the apoptotic response to imatinib can be restored in polymorphism-containing cells by BH3 mimetic drugs.

### Example H7

### Retrospective Analysis

Next, we performed a retrospective analysis on the influence of the polymorphism on TKI responses in East-Asian CML patients. Using a group of newly diagnosed chronic phase CML patients from two independent East-Asian (Singapore/Malaysia and Japan) cohorts (n=203), we compared clinical responses to first-line therapy with standard dose imatinib (400 mg/day) between individuals with and without the deletion polymorphism. Clinical responses were classified according to the European LeukemiaNet (ELN) criteria (Table H8)², and resistant patients were defined as ‘suboptimal responders’ or ‘failures’ per ELN criteria, while sensitive patients corresponded to ELN-defined ‘optimal responders’.

In both geographic cohorts, patients with the deletion polymorphism were more likely to have resistant versus sensitive disease, compared to controls (Table 1). When analyzed together, the overall odds ratio for resistant disease among patients with the deletion polymorphism versus those without was 2.94 (p=0.02, 95% CI of 1.17-7.43). By comparison, we found no significant differences between the two groups with respect to other potential prognostic or confounding factors, including median time from diagnosis to initiation of imatinib, Sokal score at diagnosis, or prior use of interferon (Table H9).

While anecdotal, we also noted that the majority of resistant patients with the polymorphism subsequently failed to respond to second-generation TKI therapy (Table H10), a finding that is in line with the intrinsic resistance we observed in cell lines.

TKI resistance is most commonly associated with the acquisition of somatic mutations in the BCR-ABL kinase domain⁷. However, because the deletion polymorphism is associated with intrinsic TKI resistance (FIG. 16), we predicted that such patients would be resistant in the absence of a kinase domain mutation. Accordingly, we divided the patients into the following three clinical groups: resistant without a BCR-ABL mutation (Group I), resistant with a BCR-ABL mutation (Group II), or sensitive (Group III). We found that patients with the polymorphism, compared to those without, were more likely to be in Group I versus Group II and Group III combined (odds ratio=1.90, 95% CI of 2.08-4.35). (Table H11). These data provide a second clinical validation of our hypothesis.

### Example H8

### Discussion (Examples H1 to H6)

Together, our findings demonstrate the principle that, while cancers should be classified according to their somatically acquired ‘driver’ mutations, germline polymorphisms can directly modulate the response of such cancers to ‘targeted’ therapies, and influence clinical outcomes. Importantly, we show how a common BIM deletion polymorphism can account, in part, for the heterogeneous responses seen in a uniformly-defined and treated group of CML patients. Strikingly, the polymorphism is found only in individuals of East-Asian countries for which a higher rate of incomplete cytogenetic responses to imatinib have been reported (˜50%) when compared to Europe and North America (26%)⁸. To assess the relative contribution of the deletion polymorphism to these ethnic differences, we estimated that the polymorphism underlies resistance in ˜23% of East-Asian cases (population attributable fraction). This might explain in part the differences in complete cytogenetic response rates observed between these two world populations.

By elucidating the effects of the deletion polymorphism on BIM function, we also describe a novel splicing mechanism by which the polymorphism contributes to drug-resistance in CML. Thus, in demonstrating that resistance is due to impaired expression of BH3-containing BIM isoforms, we were able to confirm that pharmacologic restoration of BIM function could overcome this particular form of TKI resistance. We note that while the presence of the deletion polymorphism is strongly associated with clinical TKI resistance, other genetic factors, both acquired and inherited, will likely dictate the final response to TKI therapy in CML patients. Nevertheless, screening for this polymorphism may be useful in the management of CML patients of East-Asian ancestry to predict resistance to TKIs.

Because BIM expression is required for TKI sensitivity in a broad range of human cancers²⁰⁻²³, we also investigated the influence of the BIM deletion polymorphism in epidermal growth factor receptor (EGFR)-driven non-small-cell lung cancer (NSCLC). As described in an accompanying article, we found that the deletion polymorphism results in intrinsic resistance to EGFR inhibitors in NSCLC cell lines, as well as predicts for significantly shorter progression free survival to EGFR inhibitors in NSCLC patients known to have activating EGFR mutations²⁴. Together, our data clearly demonstrate that the BIM deletion polymorphism is a germline biomarker for in vitro and in vivo TKI resistance in kinase-driven human cancers.

Finally, while the ethnic segregation of the polymorphism is in itself interesting, the greater significance of our finding may be that it is prototypic of other polymorphisms, yet to be discovered, that account for intrinsic drug resistance in different world populations.

### Example H9

### References for Examples H3 to H8

The following are the references for Example H3 to Example H8.
1. Hochhaus, A., et al. Six-year follow-up of patients receiving imatinib for the first-line treatment of chronic myeloid leukemia. Leukemia 23, 1054-1061 (2009).
2. Baccarani, M., et al. Chronic myeloid leukemia: an update of concepts and management recommendations of European LeukemiaNet. J Clin Oncol 27, 6041-6051 (2009).
3. Youle, R. J. & Strasser, A. The BCL-2 protein family: opposing activities that mediate cell death. Nat Rev Mol Cell Biol 9, 47-59 (2008).
4. Kuroda, J., et al. Bim and Bad mediate imatinib-induced killing of Bcr/Abl+leukemic cells, and resistance due to their loss is overcome by a BH3 mimetic. Proc Natl Acad Sci USA 103, 14907-14912 (2006).
5. Aichberger, K. J., et al. Low-level expression of proapoptotic Bcl-2-interacting mediator in leukemic cells in patients with chronic myeloid leukemia: role of BCR/ABL, characterization of underlying signaling pathways, and reexpression by novel pharmacologic compounds. Cancer Res 65, 9436-9444 (2005).
6. Kuribara, R., et al. Roles of Bim in apoptosis of normal and Bcr-Abl-expressing hematopoietic progenitors. Mol Cell Biol 24, 6172-6183 (2004).
7. La Rosee, P. & Hochhaus, A. Resistance to imatinib in chronic myelogenous leukemia: mechanisms and clinical implications. Curr Hematol Malig Rep 3, 72-79 (2008).
8. Au, W. Y., et al. Chronic myeloid leukemia in Asia. Int J Hematol 89, 14-23 (2009).
9. Fullwood, M. J., Wei, C. L., Liu, E. T. & Ruan, Y. Next-generation DNA sequencing of paired-end tags (PET) for transcriptome and genome analyses. Genome Res 19, 521-532 (2009).
10. Hillmer, A. M., et al. Comprehensive long-span paired-end-tag mapping reveals characteristic patterns of structural variations in epithelial cancer genomes. Genome Res (2011).
11. Liu, J. W., Chandra, D., Tang, S. H., Chopra, D. & Tang, D. G. Identification and characterization of Bimgamma, a novel proapoptotic BH3-only splice variant of Bim. Cancer Res 62, 2976-2981 (2002).
12. Carstens, R. P., McKeehan, W. L. & Garcia-Blanco, M. A. An intronic sequence element mediates both activation and repression of rat fibroblast growth factor receptor 2 pre-mRNA splicing. Mol Cell Biol 18, 2205-2217 (1998).
13. Cheng, E. H., et al. BCL-2, BCL-X(L) sequester BH3 domain-only molecules preventing BAX- and BAK-mediated mitochondrial apoptosis. Mol Cell 8, 705-711 (2001).
14. Huang, D. C. & Strasser, A. BH3-Only proteins-essential initiators of apoptotic cell death. Cell 103, 839-842 (2000).
15. Kubonishi, I. & Miyoshi, I. Establishment of a Ph1 chromosome-positive cell line from chronic myelogenous leukemia in blast crisis. Int J Cell Cloning 1, 105-117 (1983).
16. Adachi, M., Zhao, X. & Imai, K. Nomenclature of dynein light chain-linked BH3-only protein Bim isoforms. Cell Death Differ 12, 192-193 (2005).
17. Mahon, F. X., et al. Selection and characterization of BCR-ABL positive cell lines with differential sensitivity to the tyrosine kinase inhibitor STI571: diverse mechanisms of resistance. Blood 96, 1070-1079 (2000).
18. Deininger, M. W., Goldman, J. M., Lydon, N. & Melo, J. V. The tyrosine kinase inhibitor CGP57148B selectively inhibits the growth of BCR-ABL-positive cells. Blood 90, 3691-3698 (1997).
19. Cragg, M. S., Harris, C., Strasser, A. & Scott, C. L. Unleashing the power of inhibitors of oncogenic kinases through BH3 mimetics. Nat Rev Cancer 9, 321-326 (2009).
20. Cragg, M. S., Kuroda, J., Puthalakath, H., Huang, D. C. & Strasser, A. Gefitinib-induced killing of NSCLC cell lines expressing mutant EGFR requires BIM and can be enhanced by BH3 mimetics. PLoS Med 4, 1681-1689; discussion 1690 (2007).
21. Costa, D. B., et al. BIM mediates EGFR tyrosine kinase inhibitor-induced apoptosis in lung cancers with oncogenic EGFR mutations. PLoS Med 4, 1669-1679; discussion 1680 (2007).
22. Gordon, P. M. & Fisher, D. E. Role for the proapoptotic factor BIM in mediating imatinib-induced apoptosis in a c-KIT-dependent gastrointestinal stromal tumor cell line. The Journal of biological chemistry 285, 14109-14114.
23. Will, B., et al. Apoptosis induced by JAK2 inhibition is mediated by Bim and enhanced by the BH3 mimetic ABT-737 in JAK2 mutant human erythroid cells. Blood 115, 2901-2909.
24. Ng, K. P., et al. Shortened progression free survival associated with a BIM deletion polymorphism in EGFR-driven non-small-cell lung cancer. In review (2011).

### Examples N1 to N4

Examples N1 to N4 demonstrate shortened progression free survival associated with a BIM deletion polymorphism in EGFR-driven non-small-cell lung cancer.

### Example N1

### Materials and Methods

Ethics Committee Approval

Clinical NSCLC samples were obtained from patients seen at the National Cancer Centre, Singapore, Toho University Omori Medical Center, Japan, Aichi Cancer Center, Japan, and the National University Cancer Institute, National University Health System, Singapore. Written informed consent and institutional review board approval at the participating institutions were obtained from all patients who contributed samples to this study.

Cell Culture Conditions and Reagents

NSCLC cells (PC9 and HCC2279) were grown in RPMI-1640 medium supplemented with penicillin/streptomycin, glutamine and 10% FBS, and incubated in a humidified incubator at 37° C. with 5% CO₂. Gefitinib and ABT-737 were resuspended in DMSO and kept at −20° C.

Real-time RT-PCR (Q-PCR)

Total cellular RNA was extracted using the RNeasy Mini Kit (Qiagen) according to the manufacturer's instruction. RNA was reverse transcribed using Superscript III First-Strand Synthesis System (Invitrogen), and quantitatively assessed using the iQ5 Multicolor Real-Time Detection System (Bio-Rad) with a total reaction volume of 25 μl. Primers were annealed at 59° C. for 20 seconds and the amplicon was extended at 72° C. for 30 seconds. The total number of cycles quantified was 40. Transcript levels of β-actin or exon 2A of BIM were used to normalize between samples. The following primers were used: BIM exon 2A (Forward: ATGGCAAAGCAACCTTCTGATG; Reverse: GGCTCTGTCTGTAGGGAGGT), BIM exon 3 (Forward: CAATGGTAGTCATCCTAGAGG; Reverse: GACAAAATGCTCAAGGAAGAGG), BIM exon 4 (Forward: TTCCATGAGGCAGGCTGAAC; Reverse: CCTCCTTGCATAGTAAGCGTT) and β-actin (Forward: GGACTTCGAGCAAGAGATGG; Reverse: AGCACTGTGTTGGCGTAC-AG).

Western Blot

Western blotting was performed using antibodies against human BIM, CASPASE 3, cleaved CASPASE 3, PARP, phospho-EGFR (Y1068) (all from Cell Signaling Technology), and β-actin (Sigma). Detection was performed with HRP-conjugated secondary antibodies that are specific to rabbit (Sigma) or mouse IgG (Santa Cruz). The membrane was visualized using the Western Lightning chemiluminescence reagent (PerkinElmer).

Trypan Blue Viability Assay

PC9 or HCC2279 cells were seeded in a 24-well plate at 5×10⁵ or 1.6×10 cells per wells in triplicate, and treated with DMSO (no drug), 0.5 μM gefitinib, ABT-737 or combination of gefitinib and ABT-737. Trypan blue staining was performed to assess the number of viable cells 48 hours post treatment.

EGFR Mutation Analysis

FFPE slides of lung tumors were deparaffined by washing the slides in xylene and absolute ethanol. Lung cancer regions from each slide were scraped, transferred into a 1.5 ml tube, and genomic DNA was extracted using a QIAGEN QIAamp FFPE Tissue kit. EGFR exons 18 to 21 were sequenced. 50 ng of FFPE gDNA was amplified by PCR in a 20 ul reaction volume containing 10 μl of GoTaq hot start Taq colorless master mix (M5133, Promega). The PCR conditions were: 95° C. for 5 minutes, DNA amplification for 35 cytes at 95° C. for 50 seconds; 58° C. for 50 seconds; 72° C. for 60 seconds and a final extension at 72° C. for 10 minutes. The PCR primers used were: exon 18 (Forward: TGGCACTGCTTTCCAGCATGG; Reverse: CTCCCCACCAGACCATGAGAGG), exon 19 (Forward: ATCACTGGGCAGCATGTGGCA; Reverse: CCTGAGGTTCAGAGCCATGGAC), exon 20 (Forward: CATGCGAAGCCACACTGACGTG; Reverse: GCATGTGAGGATCCTGGCTC), exon 21 (Forward: GATCTGTCCCTCACAGCAGG; Reverse: GGTGTCAGGAAAATGCTGGCTG). PCR products were purified by Exonuclease I (M0293L, New England Biolabs, Inc)—Shrimp Alkaline Phosphatase (M8201, Promega) treatment. Purified PCR products were sequenced in forward and reverse directions using the ABI PRISM BigDye Terminator Cycle Sequencing Ready Reaction kit (Version 3) on an ABI PRISM 3730 Genetic Analyzer (Applied Biosystems, CA). Chromatograms were analyzed by SeqScape V2.5 and manual review.

Screening for the Polymorphic Deletion in BIM

DNA were either extracted from patients' blood samples or recovered from formalin-fixed paraffin-embedded (FFPE) biopsy slides/blocks. For genomic DNA from blood, we genotyped the deletion in the samples by single PCR reaction using primers Bim_del_F AATACCACAGAGGCCCACAG and Bim_del_R GCCTGAAGGTGCTGAGAAAG and JumpStart RedAccuTaq LA DNA Polymerase (Sigma) with the following thermo cycling conditions: 96° C. for 30 seconds, (94° C. for 15 seconds, 60° C. for 60 seconds, 68° C. for 10 minutes) ×29, 68° C. for 20 minutes. The resulting PCR products from the deletion (1,323 bp) and the wildtype (4,226 bp) alleles were analyzed on 1% agarose gels.

For DNA recovered from FFPE tissues, PCR reactions were performed independently to determine the present of the wildtype and deletion alleles. The primers BIM FFPE F5 CCACCAATGGAAAAGGTTCA and BIM FFPE R1 CTGTCATTTCTCCCCACCAC were used to genotype the wildtype allele. The primers BIM FFPE F5 CCACCAATGGAAAAGGTTCA and BIM Del FFPE R2 GGCACAGCCTCTATGGAGAA were used to genotype the deletion allele. The PCR reaction was performed using the GoTaq Hot start Polymerase (promega) with the following thermo cycling conditions: 95° C. for 5 minutes, (95° C. for 50 seconds, 58° C. for 50 seconds, 72° C. for 1 minute) ×39, 72° C. for 10 minute. The PCR products from the deletion (284 bp) and the wildtype (362 bp) alleles were analyzed on a 2% agarose gel and were sequenced to confirm the breakpoint.

EGFR NSCLC Patient Data Analysis

The primary endpoint was to examine the effect of the BIM deletion polymorphism on progression free survival (PFS) of EGFR-mutated NSCLC patients treated with EGFR TKI from East-Asian countries. PFS was calculated from the initiation of EGFR TKI therapy till either tumor progression or death from any cause. Observations were censored if TKI therapy was stopped due to side effects. P-values of the Kaplan-Meier test comparing survival curves were calculated using the Wilcoxon test and log-rank test. The t-test and Fisher's exact test were used to test for differences between clinical characteristics of the BIM deleted and wild-type populations.

Using Cox proportional hazard regression analysis, univariate and multivariate hazard ratios were generated for the following factors: age, gender, histology, smoking history, type of EGFR mutation by exon and specific mutation, stage, first- or second-line TKI therapy, race, TKI (gefitinib or erlotinib), and ECOG status. The significance level for entering variables in a stepwise regression was 0.05. SAS System for Windows Version 9.2 PHREG procedure was used to perform the calculations.

TABLE N1
Table N1. Patient characteristics according to
BIM polymorphism status

| | Without BIM | | With BIM | | |
|---|---|---|---|---|---|
| | polymorphism | | polymorphism | | |
| | (n = 115) | | (n = 26) | | |
| Characteristics | No. | % | No. | % | P-value |

| | | | |
|---|---|---|---|
| Age (in years) | | | 0.7521 |
| Mean | 59.0 | 59.7 | |
| SD | 10.4 | 9.8 | |
| Range | 33-80 | 39-80 | |

| | | | | | |
|---|---|---|---|---|---|
| Sex | | | | | 0.4980 |
| Male | 40 | 34.8 | 7 | 26.9 | |
| Female | 75 | 65.2 | 19 | 73.1 | |
| Smoking status | | | | | 0.8973 |
| Current smoker | 5 | 4.3 | 1 | 3.8 | |
| Ex-smoker | 15 | 13.0 | 4 | 15.4 | |
| Never | 94 | 81.7 | 20 | 76.9 | |
| Unknown | 1 | 0.9 | 1 | 3.8 | |
| Stage | | | | | 0.6990 |
| III | 10 | 8.7 | 1 | 3.8 | |
| IV | 99 | 86.1 | 24 | 92.3 | |
| Recurrent | 6 | 5.2 | 1 | 3.8 | |
| Histology | | | | | 0.1663 |
| Adenocarcinoma | 104 | 90.4 | 24 | 92.3 | |
| Bronchioalveolar carcinoma | 2 | 1.7 | 2 | 7.7 | |
| Squamous cell carcinoma | 0 | 0.0 | 0 | 0.0 | |
| Others | 9 | 7.8 | 0 | 0.0 | |
| EGFR exon mutated | | | | | 0.3360 |
| 18 | 4 | 3.5 | 1 | 3.8 | |
| 19 | 66 | 57.4 | 11 | 42.3 | |
| 20 | 5 | 4.3 | 1 | 3.8 | |
| 21 | 37 | 32.2 | 13 | 50.0 | |
| 19 and 20 | 2 | 1.7 | 0 | 0.0 | |
| 18 and 21 | 1 | 0.9 | 0 | 0.0 | |
| EGFR mutation | | | | | 0.6134 |
| G719_(Exon 18) | 4 | 3.5 | 1 | 3.8 | |
| Exon 19 deletion | 64 | 55.7 | 11 | 42.3 | |
| L858R (Exon 21) | 37 | 32.2 | 12 | 46.2 | |
| Others | 7 | 6.1 | 2 | 7.7 | |
| Double mutation | 3 | 2.6 | 0 | 0.0 | |
| TKI Used | | | | | 0.2292 |
| Gefitinib only | 112 | 97.4 | 24 | 92.3 | |
| Erlotinib only | 3 | 2.6 | 2 | 7.7 | |
| Line of treatment | | | | | 1.0000 |
| First-line | 76 | 66.1 | 17 | 65.4 | |
| Second-line or later | 38 | 33.0 | 9 | 34.6 | |
| Unknown | 1 | 0.9 | 0 | 0.0 | |
| Cohort | | | | | 1.0000 |
| Singapore | 95 | 82.6 | 22 | 84.6 | |
| Japan | 20 | 17.4 | 4 | 15.4 | |
| Race | | | | | 0.9277 |
| Chinese | 82 | 71.3 | 20 | 76.9 | |
| Malay | 9 | 7.8 | 1 | 3.8 | |
| Indian | 1 | 0.9 | 0 | 0.0 | |
| Japanese | 20 | 17.4 | 4 | 15.4 | |
| Others | 3 | 2.6 | 1 | 3.8 | |
| ECOG performance status | | | | | 0.0801 |
| 0-1 | 94 | 81.7 | 21 | 80.8 | |
| 2 | 6 | 5.2 | 4 | 15.4 | |
| 3 | 6 | 5.2 | 0 | 0.0 | |
| Unknown | 9 | 7.8 | 1 | 3.8 | |

### Example N2

### Introduction

Many but not all non-small-cell lung cancer (NSCLC) patients with activating mutations in the epidermal growth factor receptor (EGFR) respond to EGFR inhibitors. However, an initial response to EGFR inhibitors does not predict for response duration. We report that a novel BIM deletion polymorphism impairs the pro-apoptotic BIM response to EGFR inhibitors in cell lines, and predicts for considerably shortened progression free survival in patients.

Activating mutations in EGFR predict for high response rates among patients with NSCLC^{1,2}. Such cancers are particularly common in East-Asian countries where EGFR mutations can be found in up to 50% of non-small-cell lung cancers, and interestingly are more common among female, East-Asian, non-smokers³⁻⁵. In contrast, activating EGFR mutations can only be found in about 10% of lung cancers in the West. However, regardless of patient ethnicity, therapy with EGFR tyrosine kinase inhibitors (TKI) in tumors harboring activating mutations is associated with higher response rates⁶⁻⁸, and significantly prolonged progression free survival compared with cytoxic chemotherapy⁹. However, despite high overall response rates, considerable heterogeneity exists with respect to response duration (weeks to years) and degree of tumor shrinkage (complete response to stable disease)¹⁰⁻¹².

These clinical observations suggest that genetic or epigenetic modifiers might regulate tumor responses to TKI therapy, even when there is effective inhibition of the underlying ‘driver’ mutation, e.g. the BCR-ABL or EGFR kinases. Using a genome-wide paired-end sequencing approach, we recently discovered a deletion polymorphism in the BIM gene that resulted in intrinsic imatinib resistance in chronic myelogenous leukemia cell lines, and inferior clinical responses to imatinib¹³. Strikingly, the polymorphism was restricted to individuals of East-Asian ancestry (12.3% carrier rate)¹³, which as described above, is an intriguing demographic that enriches for NSCLC patients with sensitizing EGFR mutations. Mechanistically, the deletion biases splicing away from BIM isoforms containing exon 4, which encodes the pro-death BH3 domain, and thereby impairs the ability of TKIs to upregulate BH3-containing BIM protein isoforms. This in turn leads to a blunted apoptotic response following TKI exposure, and intrinsic resistance to TKIs¹³. Because EGFR inhibitors also require induction of BH3-containing BIM isoforms to activate apoptosis^{14,1517,18}, and preventing BIM induction rendered EGFR mutated lung cancer cells resistant to TKIs^{16,17}, we asked if the BIM deletion polymorphism also impaired TKI responses in NSCLC with EGFR mutations.

### Example N3

### HCC2779 has TKI-Sensitizing EGFR Mutations but is TKI Resistant

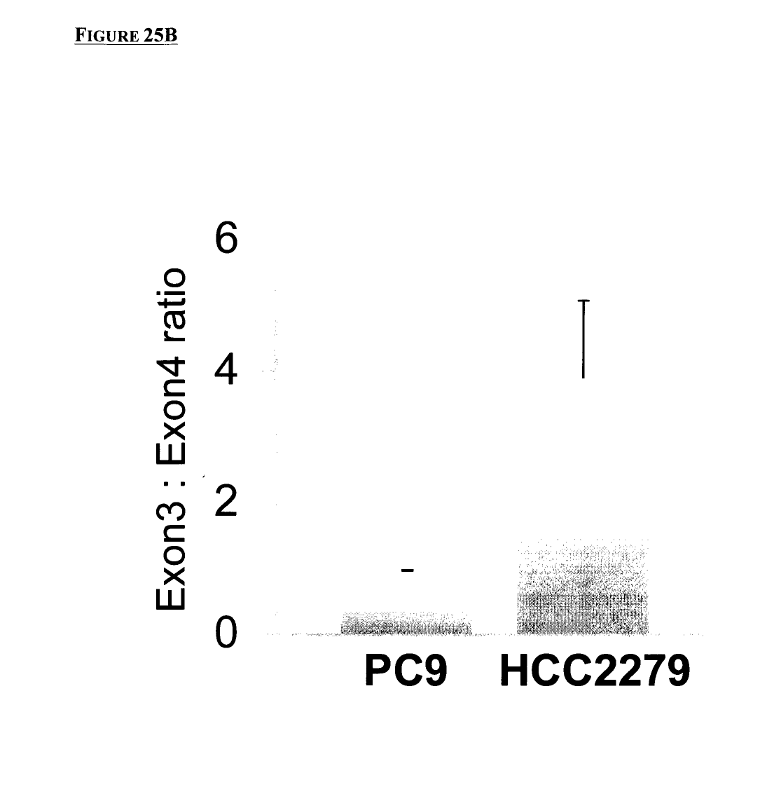

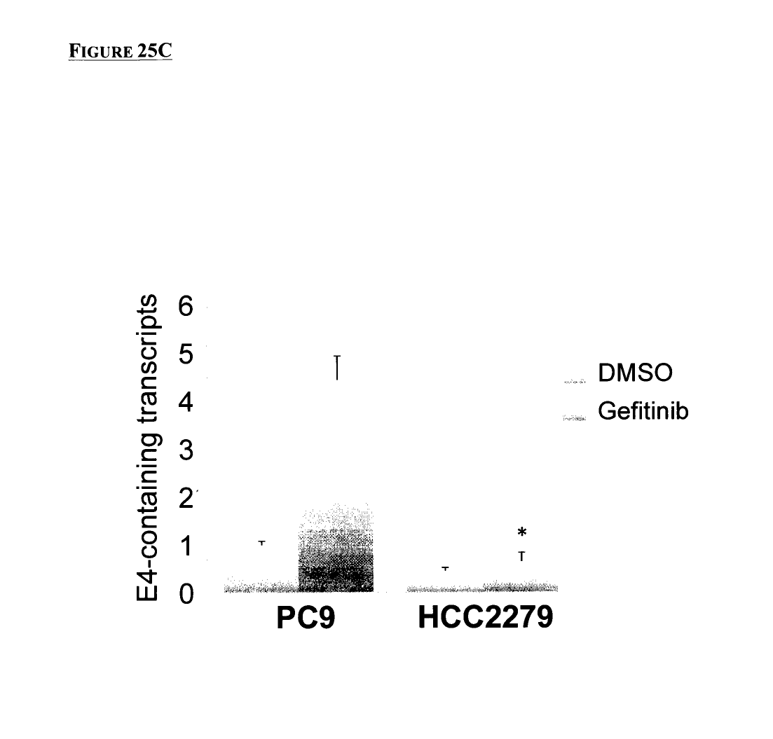

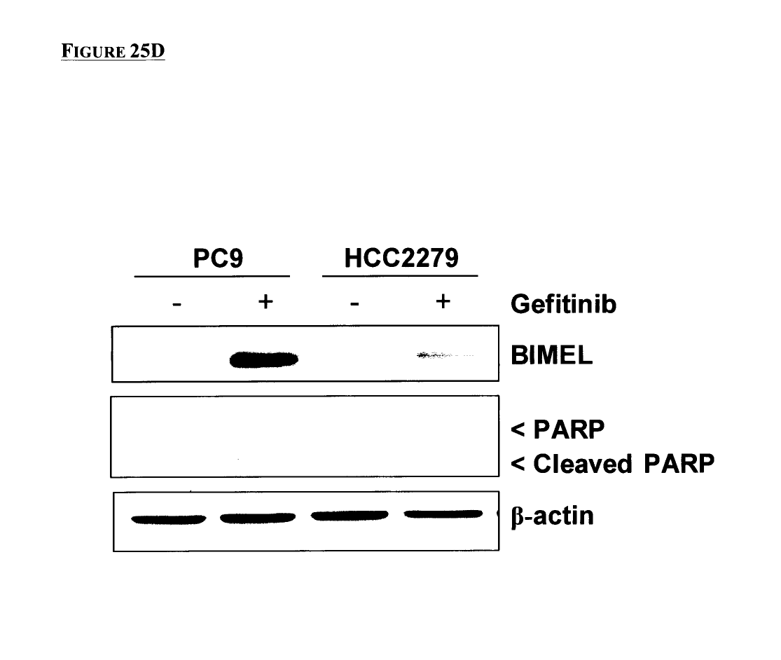

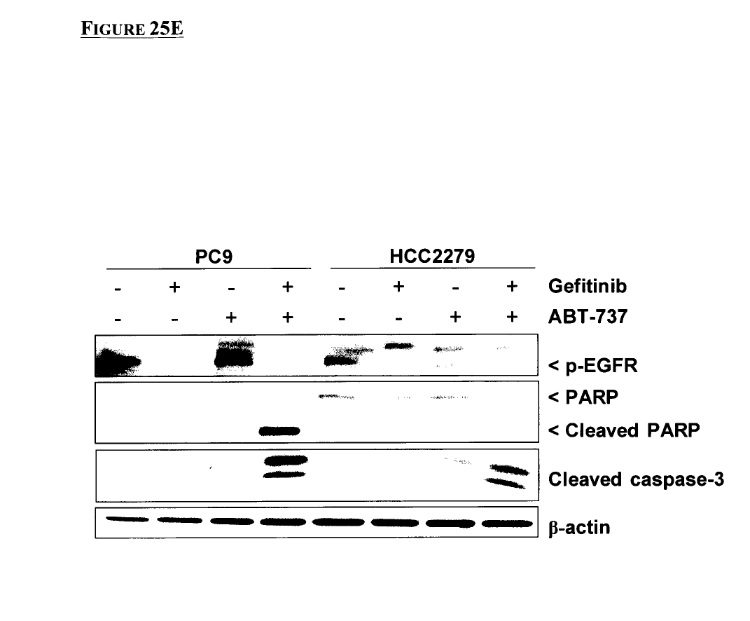

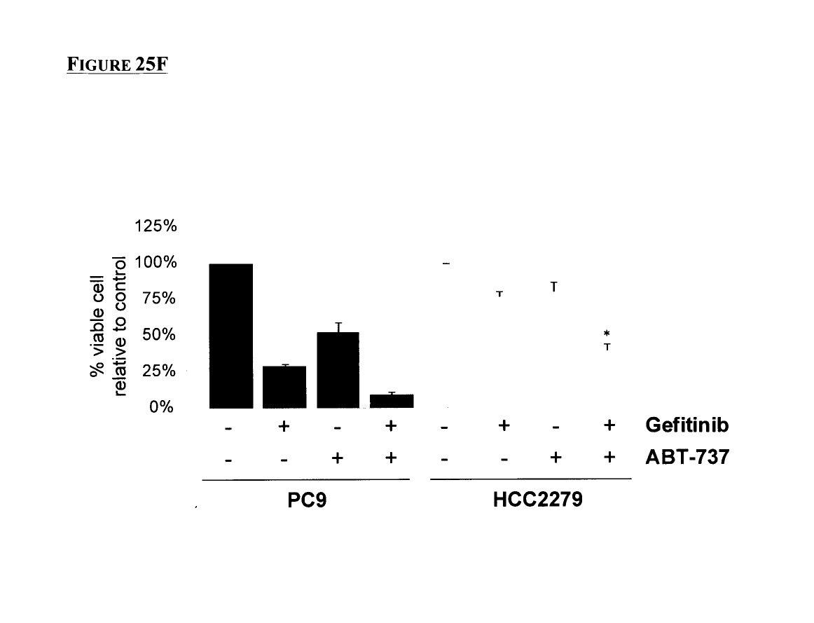

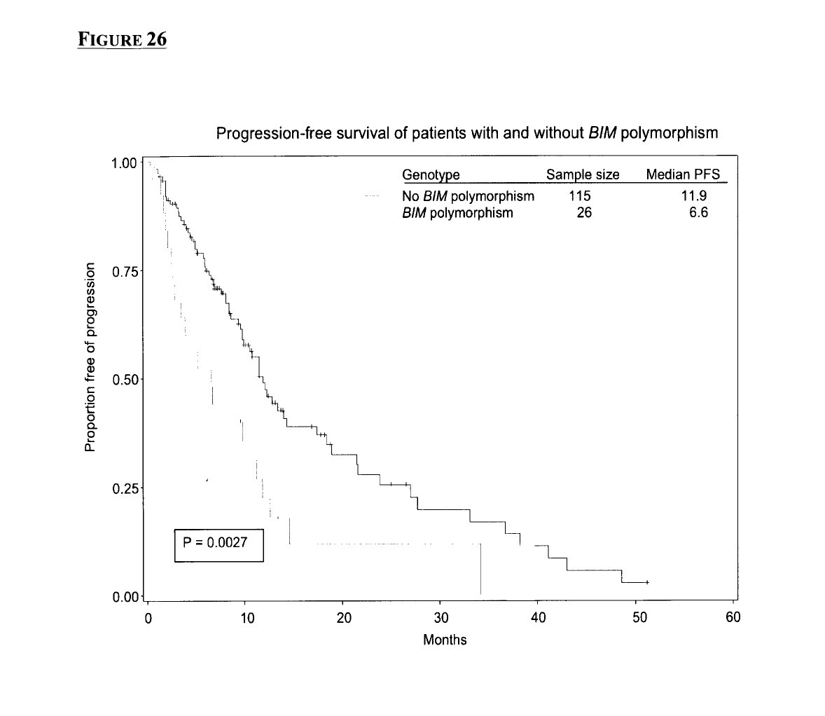

To help answer this question, we searched for NSCLC cell lines that harbored TKI-sensitizing EGFR mutations but were inexplicably TKI resistant (defined as lacking any of the known secondary resistance-conferring mutations). We were able to identify one such line, HCC2279, which notably, fails to activate apoptosis despite effective EGFR inhibition^{16,1719,20}. We confirmed the presence of the BIM deletion polymorphism in HCC2779 cells (FIG. 25A), and determined the effects of the deletion polymorphism on BIM function. As expected, the deletion resulted in increased expression of exon 3 versus exon 4/BH3-containing BIM isoforms compared to cells without the polymorphism (FIG. 25B). In addition, TKI exposure was accompanied by decreased expression of exon 4/BH3-containing BIM isoform at the transcript and protein levels compared to control cells, as well as impaired activation of apoptotic signaling (FIG. 25C and FIG. 25D). Consistent with the notion that TKI resistance is due to decreased levels of BH3-containing BIM protein, the addition of the BH3 mimetic drug, ABT-737, enhanced TKI-induced apoptotic signaling and cell death (FIG. 25E and FIG. 25F). Separately, we have also shown that de novo introduction of the deletion polymorphism into imatinib sensitive CML cells is sufficient to render such cells intrinsically imatinib resistant¹⁶, suggesting that the polymorphism is largely responsible for TKI resistance in HCC2279 cells.

### Example N4

### Deletion in BIM Correlates with Duration of Response to EGFR TKIs

Next, we asked if the deletion correlated with the duration of response to EGFR TKIs in NSCLC patients with activating EGFR mutations. Patients with or without the deletion polymorphism did not differ with respect to known prognostic factors (Table N1). Nevertheless, the presence of the polymorphism was predictive of a significantly shorter progression free survival, with a median of 6.6 months compared to 11.9 months for individuals without the polymorphism (n=141, p=0.0027) (FIG. 26). In multivariate analyses using the Cox regression model, only the deletion polymorphism (hazard ratio=2.14, 95% confidence interval 1.30-3.50, p=0.0026), in addition to the presence of the TKI-resistant exon 20 mutation (hazard ratio=6.00, 95% confidence interval 2.05-17.57, p=0.0011)^{18,19}, emerged as independent prognostic factors for poorer progression free survival.

In summary, we find that the BIM deletion polymorphism results in impaired splicing and expression of BH3-containing BIM isoforms in normal and cancer cells (FIG. 25 and reference 16). In the setting of tyrosine kinase-driven cancers, the aberrant splicing conferred by the polymorphism is sufficient to result in intrinsic cellular resistance to TKIs, regardless of the underlying ‘driver’ mutation (FIG. 25 and reference 16). Importantly, in patients with either CML or EGFR-mutated NSCLC, the presence of the polymorphism is also associated with significantly inferior TKI responses (FIG. 26 and reference 16).

Within the context of targeted cancer therapies, our results offer an explanation for the heterogeneity of tumor responses among homogenously-defined and -treated patients. Our data also highlight how a single germline polymorphism can, when it occurs in a critical gene, strongly impact clinical outcomes in different cancers sharing a common biology. This likely reflects the central role of BIM in mediating TKI sensitivity in these diseases^{14,15,20}. We anticipate that the list of cancers in which the BIM polymorphism influences TKI responses will expand to include others that also depend on BIM expression for sensitivity²¹⁻²³. While our data focuses on the effect of polymorphisms on therapeutic responses, it is possible that human polymorphisms also account for heterogeneity among other aspects of cancer biology. Unlike the BIM deletion, these could conceivably result in enhanced therapeutic responses, or even cooperate with ‘driver’ mutations to accelerate or delay cancer progression. As we have demonstrated, a mechanistic understanding of how such polymorphisms affect gene function may lead to improved management of cancer patients with respect to prognostication and therapy. In the case of TKI resistance in patients with the BIM polymorphism, the addition of BH3 mimetics to standard TKI therapy may allow for personalized treatment to overcome resistance.

### Example N5

### References for Examples N2 to N4

The following are the references for Example N2 to Example N4.
1. Paez, J. G., et al. Science 304, 1497-1500 (2004).
2. Lynch, T. J., et al. N Engl J Med 350, 2129-2139 (2004).
3. Shepherd, F. A., et al. N Engl J Med 353, 123-132 (2005).
4. Kim, E. S., et al. Lancet 372, 1809-1818 (2008).
5. Park, K. & Goto, K. Curr Med Res Opin 22, 561-573 (2006).
6. Inoue, A., et al. J Clin Oncol 24, 3340-3346 (2006).
7. Rosell, R., et al. N Engl J Med 361, 958-967 (2009).
8. Sequist, L. V., et al. J Clin Oncol 26, 2442-2449 (2008).
9. Mok, T. S., et al. N Engl J Med 361, 947-957 (2009).
10. Gazdar, A. F. Oncogene 28 Suppl 1, S24-31 (2009).
11. Sharma, S. V., Bell, D. W., Settleman, J. & Haber, D. A. Nat Rev Cancer 7, 169-181 (2007).
12. Sequist, L. V., Bell, D. W., Lynch, T. J. & Haber, D. A. J Clin Oncol 25, 587-595 (2007).
13. Hillmer, A. M., et al. In review (2011).
14. Costa, D. B., et al. PLoS Med 4, 1669-1679; discussion 1680 (2007).
15. Cragg, M. S., Kuroda, J., Puthalakath, H., Huang, D. C. & Strasser, A. PLoS Med 4, 1681-1689; discussion 1690 (2007).
16. Machida, K., et al. PLoS One 5, e13470 (2010).
17. Lu, Y., Liang, K., Li, X. & Fan, Z. Mol Cancer 6, 63 (2007).
18. Wu, J. Y., et al. Clin Cancer Res 14, 4877-4882 (2008).
19. Sasaki, H., et al. Lung Cancer 58, 324-328 (2007).
20. Kuroda, J., et al. Proc Natl Acad Sci USA 103, 14907-14912 (2006).
21. Gordon, P. M. & Fisher, D. E. J Biol Chem 285, 14109-14114 (2010).
22. Will, B., et al. Blood 115, 2901-2909 (2010).
23. Soda, M., et al. Nature 448, 561-566 (2007).

Each of the applications and patents mentioned in this document, and each document cited or referenced in each of the above applications and patents, including during the prosecution of each of the applications and patents (“application cited documents”) and any manufacturer's instructions or catalogues for any products cited or mentioned in each of the applications and patents and in any of the application cited documents, are hereby incorporated herein by reference. Furthermore, all documents cited in this text, and all documents cited or referenced in documents cited in this text, and any manufacturer's instructions or catalogues for any products cited or mentioned in this text, are hereby incorporated herein by reference.

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the claims.

1. A method of predicting whether an individual susceptible to or suffering from cancer or a myeloproliferative disorder is likely to develop resistance to treatment with a tyrosine kinase inhibitor, the method comprising detecting whether the individual has a polymorphic variant of a BIM (BCL2L11) gene which comprises, in 5′ to 3′ order, the nucleotide sequence set out in SEQ ID NO: 5 followed immediately by the nucleotide sequence set out in SEQ ID NO: 7.

2. The method according to claim 1, which comprises: (a) detecting the presence of a nucleic acid amplification product comprising a sequence set out in SEQ ID NO: 1 as an indicator of the individual having the polymorphic variant of the BIM (BCL2L11) gene, or (b) detecting the presence of a nucleic acid amplification product comprising a sequence set out in SEQ ID NO: 2 as an indicator of the individual lacking the polymorphic variant of the BIM (BCL2L11) gene.

3. The method according to claim 1, in which the method comprises nucleic acid amplification using a nucleotide sequence (a) as set out in SEQ ID NO: 3, or a (b) as set out in SEQ ID NO: 4, or a combination of (a) and (b).

4. The method according to claim 1, in which (a) if the individual is determined to have the polymorphic variant of the BIM (BCL2L11) gene, then the individual is likely to develop resistance to treatment with a tyrosine kinase inhibitor, or in which (b) if the individual is determined not to have the polymorphic variant of the BIM (BCL2L11) gene, then the individual is less likely to develop resistance to treatment with a tyrosine kinase inhibitor.

5. A method of choosing a therapy for an individual with cancer or a myeloproliferative disorder, the method comprising determining whether a patient is likely to develop resistance to treatment with a tyrosine kinase inhibitor by a method comprising determining whether the individual has a polymorphic variant of a BIM (BCL2L11) gene which comprises, in 5′ to 3′ order, the nucleotide sequence set out in SEQ ID NO: 5 followed immediately by the nucleotide sequence set out in SEQ ID NO: 7 and where the individual is determined as being likely to develop such resistance, choosing a therapy comprising any one or more of the following:
(a) more frequent monitoring of the patient;
(b) more frequent blood and bone marrow tests;
(c) bone marrow transplantation;
(d) administration of a more potent tyrosine kinase inhibitor (TKI);
(e) administration of a BH3-mimetic; or
(g) treatment with a drug that inhibits the pro-survival effect of the BCL2 group of proteins.

6. (canceled)

7. The method according to claim 1, in which:
(a) the cancer or myeloproliferative disorder comprises chronic myelogenous leukaemia (CML) and in which the resistance to treatment with a tyrosine kinase inhibitor comprises BCR-ABL-independent TKI-resistance;
(b) the cancer or myeloproliferative disorder comprises gastrointestinal stromal tumour (GIST), and in which the resistance to treatment with a tyrosine kinase inhibitor comprises c-KIT/PDGFR-independent TKI-resistance;
(c) the cancer or myeloproliferative disorder comprises non-small cell lung cancer (NSCLC), and in which the resistance to treatment with a tyrosine kinase inhibitor comprises EGFR-independent TKI-resistance;
(d) the cancer or myeloproliferative disorder comprises a myeloproliferative disorder selected from the group consisting of: polycythaemia vera, essential thrombocythaemia, and primary myelofibrosis, and in which the resistance to treatment with a tyrosine kinase inhibitor comprises JAK2-independent TKI-resistance, such as resistance to JAK inhibitors; or
(e) in which the cancer or myeloproliferative disorder is selected from the group consisting of: haematologic malignancies, chronic lymphocytic leukaemia, acute lymphoblastic leukaemia, acute myeloid leukaemia, multiple myeloma, myeloproliferative disorders solid tumours, small cell lung cancer, breast cancer, colorectal cancer, ovarian cancer, melanoma and neuroblastoma.

8. (canceled)

9. (canceled)

10. (canceled)

11. (canceled)

12. (canceled)

13. A method of detecting the presence or absence of a BIM (BCL2L11) polymorphism in an individual, which comprises, in 5′ to 3′ order, the nucleotide sequence set out in SEQ ID NO: 5 followed immediately by the nucleotide sequence set out in SEQ ID NO: 7, the method comprising detecting a nucleic acid amplification product comprising a sequence set out in SEQ ID NO: 1 or a sequence set out in SEQ ID NO: 2.

14. A method of treatment of a patient suffering from cancer or a myeloproliferative disorder, the method comprising determining whether the cancer or myeloproliferative disorder is a BCR-ABL-independent TKI-resistant CML cancer, a c-KIT/PDGFR-independent TKI-resistant GIST cancer, an EGFR-independent TKI-resistant NSCLC cancer or a JAK2-independent TKI-resistant myeloproliferative disorder by a method comprising determining whether the individual has a polymorphic variant of a BIM (BCL2L11) gene which comprises, in 5′ to 3′ order, the nucleotide sequence set out in SEQ ID NO: 5 followed immediately by the nucleotide sequence set out in SEQ ID NO: 7, and treating the patient by performing a step selected from (a) to (g):
(a) more frequent monitoring of the patient;
(b) more frequent blood and bone marrow tests;
(c) bone marrow transplantation;
(d) administration of a more potent tyrosine kinase inhibitor (TKI);
(e) administration of a BH3-mimetic;
(f) increasing the dose of a tyrosine kinase inhibitor; or
(g) treatment with a drug that inhibits the pro-survival effect of the BCL2 group of proteins.

15. The method according to claim 14, which comprises detecting a nucleic acid amplification product comprising a sequence set out in SEQ ID NO: 1.

16. (canceled)

17. The method of claim 1, wherein the polymorphic variant of the BIM (BCL2L11) gene lacks the nucleotide sequence set out in SEQ ID NO: 6.

18. The method of claim 5, wherein the BH3-mimetic is ABT-263.

19. The method of claim 5, wherein the BH3-mimetic is administered in combination with a TKI.

20. The method of claim 5, wherein the tyrosine kinase inhibitor dose is increased beyond the standard dose of 400 mg/day.

21. The method of claim 14, wherein administration of the more potent tyrosine kinase inhibitor (TKI) is nilotinib or dasatinib.

22. The method of claim 15, wherein the detection of the nucleic acid amplification product comprising a sequence set out in SEQ ID NO: 1 is performed using a nucleotide sequence (a) as set out in SEQ ID NO: 3, or a (b) as set out in SEQ ID NO: 4, or a combination of (a) and (b).

23. The method of claim 13, wherein the amplification product is produced using primers comprising sequences as set out in SEQ ID NO: 3 and SEQ ID NO: 4.

24. A solid support comprising a first nucleic acid that specifically hybridizes to a nucleic acid molecule comprising SEQ ID NO: 1 and a second nucleic acid molecule that specifically hybridizes to a nucleic acid molecule comprising SEQ ID NO: 2.


SEQUENCE LISTING (102 sequences)

SEQ ID NO: 1 - DNA, 1323 bp, Artificial Sequence
  Description of Artificial Sequence Synthetic polynucleotide
aataccacagaggcccacagcaaaatcatggaaggaactgacctggtggagactggtaaattggagagtatttgccccttctatgtttgggccacacctaattgtggctgtgagggcatgtggtctcagggtgggttttcctatattgcaagataacctaggaatgcaaaattgatgccagataccctgtttcaacattgacagctgattcagattttgaaaacattgtacaagctgaaaagaaacatctgcagacttgatttggcccttgaacctacagcatgtgactttgggtacatactttgggtaaccttggtgaggggctgagtctgtgttgatcgacttgctgttccccaccaatggaaaaggttcatgtcttgatcagtagtcaatcacattgaccatttgtccagattagcttgccatacatgaacaagatagaagtaagttggtagagttatcaattaggaaacccagtacagagtctattataatttagattgtacctcatgatgaaggctaactcaacaaacccatcagaacagacactggaacaaaatgacatttctaaataccatccagctctgtcttcataggcttcagtgaggtaaatcactgttctccatagaggctgtgccattttacattcccaccaacagggcacaagggttccagtttctccacatacttaccaacacttttttttttttttttttaacagtagtcatcctagaggatataggtgatctttcactgtgctttggatttatatttactggcttagatttgtatggccaccaccatagtcaagatacagaacaactcaaccacaaggatttctcatgatacctttttatagccacagccacctctctccctcttccttgagcattttgtcatatggtcattggtgattaaataaaatgtattttaatattgactttctctgtttctttctacctttttaaacatggctactagaaaaatgcacaattagatttgtggctggtgttctgtttcatctaaacaggctggcctcacagaggagctggagtgtgcagtgctgctctagcaagccaggcttgactcttcccactcagggcacatcacttccatgaagcttactccttgggttgtttggttgacttaggagaatggaagtgattagcagaatcttgtaagcattttaaacattaaatgagcattgtaaacagcggcattcttcaggcaaatacagttttgttttacctctttaaattccatggtatattcggacttcaaaaagtagatggtagagcacatgctttctcagcaccttcaggc

SEQ ID NO: 2 - DNA, 4226 bp, Artificial Sequence
  Description of Artificial Sequence Synthetic polynucleotide
aataccacagaggcccacagcaaaatcatggaaggaactgacctggtggagactggtaaattggagagtatttgccccttctatgtttgggccacacctaattgtggctgtgagggcatgtggtctcagggtgggttttcctatattgcaagataacctaggaatgcaaaattgatgccagataccctgtttcaacattgacagctgattcagattttgaaaacattgtacaagctgaaaagaaacatctgcagacttgatttggcccttgaacctacagcatgtgactttgggtacatactttgggtaaccttggtgaggggctgagtctgtgttgatcgacttgctgttccccaccaatggaaaaggttcatgtcttgatcagtagtcaatcacattgaccatttgtccagattagcttgccatacatgaacaagatagaagtaagttggtagagttatcaattaggaaacccagtacagagtctattataatttagattgtacctcatgatgaaggctaactcaacaaacccatcagaacagacactggaacaaaatgacatttctaaataccatccagctctgtcttcataggcttcagtgaggtaaatcaggcaggcctttgcccatgttatagaattggaaagaacctcagagtggtggtcacttgtcagaggttgggcacacctgtgaggtggtggggagaaatgacagacatcccagcagctacacatgctggctgcacgtctcttgccaaatgccaggaggtaattttttagggtccctccttagggaaaggggctggaagttttattattgctgttactactgctcgtgaactcatttcagccttagaagttcttggcttgtagtttttgttgtactcatgaaaatgctcccccatatatatgatcattctcgcttactataacatccttgcttactaaatgagttaacagggctttatggtgtgtatcgtgaaacacacgtgcattaaagaccctctggaaggtattagcttttcacactttcacaacaaaagcttcacacttgtggttattaagctattttctctaaccagttccctttcaagcaaaatgcatacattggtctctgtaggtgatgggttaatgcatggaaatagtttctccttccctggaactgggaatagtgggtgagatagtgtattttttaatgtaaagacaggcacaaatgctttttttgttgataaatactattttacaagctaattataagttaagcactgttacttgagatgaaatatacagggcttcaaagatcataatctaaataattatgcacagctaatggttatacctgtgaagtaaagtagtggatcctgaggtgtaattttatagtattagctgcatttcagtagatggtgtgatgaagagtttaatgcataggattaaatgagaagttacgaggagtttgtttaaagttaatgtaccgaggtaagttttcagtgttaagtttttgggagatttgttttgggagaggatgagttggggttgggggaggaaaggacttagccagatgtgagtttcttaaattgaagcataaaatttacaatttatgtagtccataattttctctggacattctacagtcttagttcatgcctgaagaccactgaaataatgctgagttgataagtggttctcttgactttgtttagtattctttactcaaccctatccatgaagttcttcaatgaagcttttgataatttattgcaaaatacattttccacaaagaagtcattatgattggtttgaactagtggaacacaaatgtgaggttataaagaggttcgccttagccaggggctcctttagctgcaaagcagttttttgctcagcaacttggggtagagatcagtgtgtcttgaagttttgttttgcaaaactttgttctaatgagaaagtcaagtcttaggaggaatgtatagtagttgagtgtttgtattaacactgttttcatattttccttttatgtctctgatttttctgaagacaagttcaaggaatatatttctctgtggggcaacagatacagttttttcacttttcctcaattttagtctccttacactctgggaggattaacttgacaaatgataccttagtgaataactgattatttttatcaaaatcactcacatgtgttggtttactgagtgcctttttggatgagtgttttatgccatatgtgtttttaatggaaattaaagtgtagtcagtacactaaagtgtagtcagtacaattggaaataagagttgagaaaagtcaggatatggaggaatgctccctagtgtcatgttagtaaatgtcttaaattttatacttgttccctggcacattggaattcacagatgggagttaatggctttctttttttttttttttttttcctcagcgtcttgtgggtacttctcttatagctggtacttgtctgacccctcctttagtttgtgagctccctgggcggggaataatggcctgcagatgctagcgagtgcctgacaaagaggagaagcccaggagatgttgagagtcagtccagctctgcctgttagcctttcagacaaataaagttgaagaaggcaggtagcaagaaaaagatcctgacctctgctctgccaaagtgtttttaattacctggatctagctgtaaggtttgccacgtagtggtgacagctgaggtctagctcagcactactcagcagggaagccacacatgcattaagcacttgacataggactagtctgaactgagttgtgctgtcattattgatacacactggattttgaggagacaaaaaagaatgcaaaatagtttaattgttttcatatgggttacatgttgaaatggtgttttaaatatataggttaaataaaatataaacttgtattgcagttaaacacaaagcgtaaaatttaccatctgaaccatttatttctaagtgtactgttcagtagtgttaagtgcacttattttgttgtgcggccaatctccagaacttcttcaccttgcaaaacagaaattctgtactcattaaacaactccccatttccccctccccccagcttctgacaaccaccattctattttctgtctattaatttgacaacttcagataccttatataagtgaaatttatatagtatttgctcttccatgacaggcttatttcacttagcgtaatgtcgtcagggttcatttatcttgcaacatgtcagaatttccttcctttttaaggctgaaggttgttccagtgtgtgtatatcacatacttcatttatccattcatccatcaggagatacttgggttgcttccactttttggctattgtgagtagtgctgctatgaacatgggtatgcaaatatcttttgggggattctgctttgaatttttttggatatatacttggaagtggaattgctggatcatatggtaattctatttttaattttttggggaaccatcatgctgttctccatagaggctgtgccattttacattcccaccaacagggcacaagggttccagtttctccacatacttaccaacacttttttttttttttttttaacagtagtcatcctagaggatataggtgatctttcactgtgctttggatttatatttactggcttagatttgtatggccaccaccatagtcaagatacagaacaactcaaccacaaggatttctcatgatacctttttatagccacagccacctctctccctcttccttgagcattttgtcatatggtcattggtgattaaataaaatgtattttaatattgactttctctgtttctttctacctttttaaacatggctactagaaaaatgcacaattagatttgtggctggtgttctgtttcatctaaacaggctggcctcacagaggagctggagtgtgcagtgctgctctagcaagccaggcttgactcttcccactcagggcacatcacttccatgaagcttactccttgggttgtttggttgacttaggagaatggaagtgattagcagaatcttgtaagcattttaaacattaaatgagcattgtaaacagcggcattcttcaggcaaatacagttttgttttacctctttaaattccatggtatattcggacttcaaaaagtagatggtagagcacatgctttctcagcaccttcaggc

SEQ ID NO: 3 - DNA, 20 bp, Artificial Sequence
  Description of Artificial Sequence Synthetic primer
aataccacagaggcccacag

SEQ ID NO: 4 - DNA, 20 bp, Artificial Sequence
  Description of Artificial Sequence Synthetic primer
gcctgaaggtgctgagaaag

SEQ ID NO: 5 - DNA, 1000 bp, Artificial Sequence
  Description of Artificial Sequence Synthetic polynucleotide
cttttgtggcagtgatgagttgaggtccaaacattagctttcaggtctgtcttcattaagctaaagtgtgttttaaccaccaggctttacatagtaatgacattttgcttgaaagggaactgatcatttacagaaaatagcttaataatcaaaagtgtaaagaaagatgacaatcatttttgaaaataacacttttaaaaaaatgaactagttcatgaaagcagtaccaacatagaaccatgaaaatggtttgttttctgcctaaattcctctttgtgcttattgctcagagggtttggacatagtactaatcagattaggttgtaggtttttatttcagggattaaaggcagtagtagggtttgaaccaagagtggctaacataaataccacagaggcccacagcaaaatcatggaaggaactgacctggtggagactggtaaattggagagtatttgccccttctatgtttgggccacacctaattgtggctgtgagggcatgtggtctcagggtgggttttcctatattgcaagataacctaggaatgcaaaattgatgccagataccctgtttcaacattgacagctgattcagattttgaaaacattgtacaagctgaaaagaaacatctgcagacttgatttggcccttgaacctacagcatgtgactttgggtacatactttgggtaaccttggtgaggggctgagtctgtgttgatcgacttgctgttccccaccaatggaaaaggttcatgtcttgatcagtagtcaatcacattgaccatttgtccagattagcttgccatacatgaacaagatagaagtaagttggtagagttatcaattaggaaacccagtacagagtctattataatttagattgtacctcatgatgaaggctaactcaacaaacccatcagaacagacactggaacaaaatgacatttctaaataccatccagctctgtcttcataggcttcagtgaggtaaatca

SEQ ID NO: 6 - DNA, 2903 bp, Artificial Sequence
  Description of Artificial Sequence Synthetic polynucleotide
ggcaggcctttgcccatgttatagaattggaaagaacctcagagtggtggtcacttgtcagaggttgggcacacctgtgaggtggtggggagaaatgacagacatcccagcagctacacatgctggctgcacgtctcttgccaaatgccaggaggtaattttttagggtccctccttagggaaaggggctggaagttttattattgctgttactactgctcgtgaactcatttcagccttagaagttcttggcttgtagtttttgttgtactcatgaaaatgctcccccatatatatgatcattctcgcttactataacatccttgcttactaaatgagttaacagggctttatggtgtgtatcgtgaaacacacgtgcattaaagaccctctggaaggtattagcttttcacactttcacaacaaaagcttcacacttgtggttattaagctattttctctaaccagttccctttcaagcaaaatgcatacattggtctctgtaggtgatgggttaatgcatggaaatagtttctccttccctggaactgggaatagtgggtgagatagtgtattttttaatgtaaagacaggcacaaatgctttttttgttgataaatactattttacaagctaattataagttaagcactgttacttgagatgaaatatacagggcttcaaagatcataatctaaataattatgcacagctaatggttatacctgtgaagtaaagtagtggatcctgaggtgtaattttatagtattagctgcatttcagtagatggtgtgatgaagagtttaatgcataggattaaatgagaagttacgaggagtttgtttaaagttaatgtaccgaggtaagttttcagtgttaagtttttgggagatttgttttgggagaggatgagttggggttgggggaggaaaggacttagccagatgtgagtttcttaaattgaagcataaaatttacaatttatgtagtccataattttctctggacattctacagtcttagttcatgcctgaagaccactgaaataatgctgagttgataagtggttctcttgactttgtttagtattctttactcaaccctatccatgaagttcttcaatgaagcttttgataatttattgcaaaatacattttccacaaagaagtcattatgattggtttgaactagtggaacacaaatgtgaggttataaagaggttcgccttagccaggggctcctttagctgcaaagcagttttttgctcagcaacttggggtagagatcagtgtgtcttgaagttttgttttgcaaaactttgttctaatgagaaagtcaagtcttaggaggaatgtatagtagttgagtgtttgtattaacactgttttcatattttccttttatgtctctgatttttctgaagacaagttcaaggaatatatttctctgtggggcaacagatacagttttttcacttttcctcaattttagtctccttacactctgggaggattaacttgacaaatgataccttagtgaataactgattatttttatcaaaatcactcacatgtgttggtttactgagtgcctttttggatgagtgttttatgccatatgtgtttttaatggaaattaaagtgtagtcagtacactaaagtgtagtcagtacaattggaaataagagttgagaaaagtcaggatatggaggaatgctccctagtgtcatgttagtaaatgtcttaaattttatacttgttccctggcacattggaattcacagatgggagttaatggctttctttttttttttttttttttcctcagcgtcttgtgggtacttctcttatagctggtacttgtctgacccctcctttagtttgtgagctccctgggcggggaataatggcctgcagatgctagcgagtgcctgacaaagaggagaagcccaggagatgttgagagtcagtccagctctgcctgttagcctttcagacaaataaagttgaagaaggcaggtagcaagaaaaagatcctgacctctgctctgccaaagtgtttttaattacctggatctagctgtaaggtttgccacgtagtggtgacagctgaggtctagctcagcactactcagcagggaagccacacatgcattaagcacttgacataggactagtctgaactgagttgtgctgtcattattgatacacactggattttgaggagacaaaaaagaatgcaaaatagtttaattgttttcatatgggttacatgttgaaatggtgttttaaatatataggttaaataaaatataaacttgtattgcagttaaacacaaagcgtaaaatttaccatctgaaccatttatttctaagtgtactgttcagtagtgttaagtgcacttattttgttgtgcggccaatctccagaacttcttcaccttgcaaaacagaaattctgtactcattaaacaactccccatttccccctccccccagcttctgacaaccaccattctattttctgtctattaatttgacaacttcagataccttatataagtgaaatttatatagtatttgctcttccatgacaggcttatttcacttagcgtaatgtcgtcagggttcatttatcttgcaacatgtcagaatttccttcctttttaaggctgaaggttgttccagtgtgtgtatatcacatacttcatttatccattcatccatcaggagatacttgggttgcttccactttttggctattgtgagtagtgctgctatgaacatgggtatgcaaatatcttttgggggattctgctttgaatttttttggatatatacttggaagtggaattgctggatcatatggtaattctatttttaattttttggggaaccatcatg

SEQ ID NO: 7 - DNA, 1000 bp, Artificial Sequence
  Description of Artificial Sequence Synthetic polynucleotide
ctgttctccatagaggctgtgccattttacattcccaccaacagggcacaagggttccagtttctccacatacttaccaacacttttttttttttttttttaacagtagtcatcctagaggatataggtgatctttcactgtgctttggatttatatttactggcttagatttgtatggccaccaccatagtcaagatacagaacaactcaaccacaaggatttctcatgatacctttttatagccacagccacctctctccctcttccttgagcattttgtcatatggtcattggtgattaaataaaatgtattttaatattgactttctctgtttctttctacctttttaaacatggctactagaaaaatgcacaattagatttgtggctggtgttctgtttcatctaaacaggctggcctcacagaggagctggagtgtgcagtgctgctctagcaagccaggcttgactcttcccactcagggcacatcacttccatgaagcttactccttgggttgtttggttgacttaggagaatggaagtgattagcagaatcttgtaagcattttaaacattaaatgagcattgtaaacagcggcattcttcaggcaaatacagttttgttttacctctttaaattccatggtatattcggacttcaaaaagtagatggtagagcacatgctttctcagcaccttcaggctgcctggagcctcccaatagaggtgtcttcgagggagtcccagctctgtctctgaaaccccaaagttacttgtttgacaccaagagaaataaggaaactttttaggtcctaagtggggagagaaagtgctagaagagaaagatatttttctttactagttccaaacacatttattaattgttagttacccaattttaaatttacatcttaaaaaaattttttttcagataattacagattcacatgcatttataggaaataatacaaagaaattgtatatgccattcaccca

SEQ ID NO: 8 - DNA, 22 bp, Artificial Sequence
  Description of Artificial Sequence Synthetic primer
atggcaaagcaaccttctgatg

SEQ ID NO: 9 - DNA, 20 bp, Artificial Sequence
  Description of Artificial Sequence Synthetic primer
ggctctgtctgtagggaggt

SEQ ID NO: 10 - DNA, 21 bp, Artificial Sequence
  Description of Artificial Sequence Synthetic primer
caatggtagtcatcctagagg

SEQ ID NO: 11 - DNA, 22 bp, Artificial Sequence
  Description of Artificial Sequence Synthetic primer
gacaaaatgctcaaggaagagg

SEQ ID NO: 12 - DNA, 20 bp, Artificial Sequence
  Description of Artificial Sequence Synthetic primer
ttccatgaggcaggctgaac

SEQ ID NO: 13 - DNA, 21 bp, Artificial Sequence
  Description of Artificial Sequence Synthetic primer
cctccttgcatagtaagcgtt

SEQ ID NO: 14 - DNA, 20 bp, Artificial Sequence
  Description of Artificial Sequence Synthetic primer
ggacttcgagcaagagatgg

SEQ ID NO: 15 - DNA, 20 bp, Artificial Sequence
  Description of Artificial Sequence Synthetic primer
agcactgtgttggcgtacag

SEQ ID NO: 16 - DNA, 23 bp, Artificial Sequence
  Description of Artificial Sequence Synthetic primer
acccactcctttctttatggacc

SEQ ID NO: 17 - DNA, 22 bp, Artificial Sequence
  Description of Artificial Sequence Synthetic primer
tgatcaggcagttggaattgtg

SEQ ID NO: 18 - RNA, 19 nt, Artificial Sequence
  Description of Artificial Sequence Synthetic oligonucleotide
ccaccauagucaagauaca

SEQ ID NO: 19 - RNA, 19 nt, Artificial Sequence
  Description of Artificial Sequence Synthetic oligonucleotide
cagaacaacucaaccacaa

SEQ ID NO: 20 - DNA, 29 bp, Artificial Sequence
  Description of Artificial Sequence Synthetic primer
gccgctcgagtctctccatgtggtgtttg

SEQ ID NO: 21 - DNA, 31 bp, Artificial Sequence
  Description of Artificial Sequence Synthetic primer
gccgaagcttcctccttgcatagtaagcgtt

SEQ ID NO: 22 - DNA, 31 bp, Artificial Sequence
  Description of Artificial Sequence Synthetic primer
gccggatatcatggaaggaactgacctggtg

SEQ ID NO: 23 - DNA, 31 bp, Artificial Sequence
  Description of Artificial Sequence Synthetic primer
gccgatcgatgtaggaaactgggtgaatggc

SEQ ID NO: 24 - DNA, 25 bp, Artificial Sequence
  Description of Artificial Sequence Synthetic primer
cataaataccacagaggcccacagc

SEQ ID NO: 25 - DNA, 27 bp, Artificial Sequence
  Description of Artificial Sequence Synthetic primer
ccctcgaagacacctctattgggaggc

SEQ ID NO: 26 - DNA, 29 bp, Artificial Sequence
  Description of Artificial Sequence Synthetic primer
ggccttcaaccactatctcagtgcaatgg

SEQ ID NO: 27 - DNA, 26 bp, Artificial Sequence
  Description of Artificial Sequence Synthetic primer
ggtttcagagacagagctgggactcc

SEQ ID NO: 28 - DNA, 149 bp, Artificial Sequence
  Description of Artificial Sequence Synthetic polynucleotide
cttaatatcatttttaaaaagtggacacatgggaggccaaggcaggcagatcatgaggtcaagagttcaagaccgcctgaccaacatggtgaaaccctgtctgtactaaaaatacaaaaaaattagccatgcgaggtggtgcacgcctg

SEQ ID NO: 29 - DNA, 133 bp, Artificial Sequence
  Description of Artificial Sequence Synthetic polynucleotide
gagtgcagatagaaggacctgcttatgcccaaccagcatccgatgacatcagcccctttcttcctgatcccctttgtccggagcagagtgatggcttttcacacctcccagggaggatgtgctcagcatcaac

SEQ ID NO: 30 - DNA, 135 bp, Artificial Sequence
  Description of Artificial Sequence Synthetic polynucleotide
gctcatgcctataatcccagcactttgggaggccgaggcaggtggatcacttgaggtcaggagttcgagaccagcctggccaacatggtgaagccccatctctactaaaaataaaaaattagccgggtgtggtgg

SEQ ID NO: 31 - DNA, 140 bp, Artificial Sequence
  Description of Artificial Sequence Synthetic polynucleotide
cgtgcacctgtgatcccagctacttgggaggctgggttgggaggatcacctgagcccaggaggcagaggttgcagtgaaccaagatcacaccactagactccagcctgggcgacagagtgagaccctgcctctttttttt

SEQ ID NO: 32 - DNA, 122 bp, Artificial Sequence
  Description of Artificial Sequence Synthetic polynucleotide
atttagattgtacctcatgatgaaggctaactcaacaaacccatcagaacagacactggaacaaaatgacatttctaaataccatccagctctgtcttcataggcttcagtgaggtaaatca

SEQ ID NO: 33 - DNA, 108 bp, Artificial Sequence
  Description of Artificial Sequence Synthetic polynucleotide
ctgttctccatagaggctgtgccattttacattcccaccaacagggcacaagggttccagtttctccacatacttaccaacacttttttttttttttttttaacagta

SEQ ID NO: 34 - DNA, 122 bp, Artificial Sequence; identical to SEQ ID NO: 32
  Description of Artificial Sequence Synthetic polynucleotide

SEQ ID NO: 35 - DNA, 108 bp, Artificial Sequence; identical to SEQ ID NO: 33
  Description of Artificial Sequence Synthetic polynucleotide

SEQ ID NO: 36 - DNA, 122 bp, Artificial Sequence; identical to SEQ ID NO: 32
  Description of Artificial Sequence Synthetic polynucleotide

SEQ ID NO: 37 - DNA, 108 bp, Artificial Sequence; identical to SEQ ID NO: 33
  Description of Artificial Sequence Synthetic polynucleotide

SEQ ID NO: 38 - DNA, 146 bp, Artificial Sequence
  Description of Artificial Sequence Synthetic polynucleotide
cttaacattttgaaagcaaagccaaatgaagctcaatcacgaaaggtcttttctttctttaaggtgagagatggagcacaaatgaagcataccatggtaaataggaactgtgctcacttgcagtcatgcactatagccattaagcc

SEQ ID NO: 39 - DNA, 154 bp, Artificial Sequence
  Description of Artificial Sequence Synthetic polynucleotide
gcatttcataaagcagtatgggtggccagtgcagggagctagagcatgagctaatggcaccagggtccggtgtaggcagttagctctgctatatttggagggtgtagattgtactcagctggcagtgtttggaatctgtctttttggtcacaaa

SEQ ID NO: 40 - DNA, 128 bp, Artificial Sequence
  Description of Artificial Sequence Synthetic polynucleotide
ttatggctgggatgtttttgagaaatttatcatatgcattgcttagtaccttaagaccttaaggcttaatggctatagtgcatgactgcaagtgagcacagttcctatttaccatggtatgcttcatt

SEQ ID NO: 41 - DNA, 122 bp, Artificial Sequence
  Description of Artificial Sequence Synthetic polynucleotide
gctattgatatttactttttaatttttttttttagatgaagtcttgctctgtcacccagggctggaatgcaatggcacgatctcggctcacttgcaacctccgccttccaggttcaagtgat

SEQ ID NO: 42 - DNA, 95 bp, Artificial Sequence
  Description of Artificial Sequence Synthetic oligonucleotide
ctgagatacttaaacacgaaaaacccccagaaatattttaatataatagatgtggtttgaacccaggtattggtatgcttcctttaagctcctca

SEQ ID NO: 43 - DNA, 99 bp, Artificial Sequence
  Description of Artificial Sequence Synthetic oligonucleotide
atgggaagaaaatagggagatataatgggtgttggtggatgttagaattccaccaaaaacacctcaaattccatttaaattgatttgatatgtaggttt

SEQ ID NO: 44 - DNA, 143 bp, Artificial Sequence
  Description of Artificial Sequence Synthetic polynucleotide
ccacaaagggaagtctctaaaaaacccttaactacagtaggtaagcgctacccccagattcttactgaagtaccaatcaagtaacaattctctggttcccttcctgctcctccctctccatagtctaactagtcctgaagata

SEQ ID NO: 45 - DNA, 107 bp, Artificial Sequence
  Description of Artificial Sequence Synthetic polynucleotide
gtggtttttggcctggctgcctagtgggatcaactgagatacttaaacacgaaaaacccccagaaatattttaatataatagatgtggtttgaacccaggtattggt

SEQ ID NO: 46 - DNA, 338 bp, Artificial Sequence
  Description of Artificial Sequence Synthetic polynucleotide
tttaaaaaaaagagtgtctcctaaagtgtaagcgctatcatgctggactaatgttttaatttaatttaattgttgtcttggtagagatggggtctcgctattttgctcacgcagatctcaaactcctgacgcccaggtacactcccatctcggcctcctaaaatggtgggatcacagacgtcagccaccgagcccggtcacttttttgtattccccacagtattgatgtatatcttctgcgttcaaaagcaattttttaaagcctcataacgtggtaacagaatactttgcacgttacaaaattcagaacacggaaacaagaagctcgcttttttttc

SEQ ID NO: 47 - DNA, 175 bp, Artificial Sequence
  Description of Artificial Sequence Synthetic polynucleotide
ccccctatttcggtttggccctttagatttcccctcccccaccggggcgggacttcccgccgacttctttcaggttctcagttcggtccgccaactgtcgtataaaggcgctgcctcaggccagagtcctcacaaagcgttgggtgagactcctcttgctcgtcatgtctggccg

SEQ ID NO: 48 - DNA, 177 bp, Artificial Sequence
  Description of Artificial Sequence Synthetic polynucleotide
attagtcctgcctacagtgaccccgggtgaatggccacacgtgtgccgctgtgttcaaaaaactgttccccccagaactgactcagaaccaagcaaaattccgtacctaccaaaaccactcctcagtgttaccactcttcagctgagaaagtagggtggccctgaaaagggcctttt

SEQ ID NO: 49 - DNA, 150 bp, Artificial Sequence
  Description of Artificial Sequence Synthetic polynucleotide
gttgatagaaagggacgctcaaccaccgaaaccgtagagggtgcggccctggcgcttgagcgcgtagaccacatccatggcggtgaccgtcttgcgcttggcgtgctctgtataggtcacggcgtcccggatcacgttctccaggaacac

SEQ ID NO: 50 - DNA, 158 bp, Artificial Sequence
  Description of Artificial Sequence Synthetic polynucleotide
aacttttgagattgatgaggggtttattttgattcagttttataatttgtgtcttttaatcaattggttcatttaatttatccaagttatggtataaaattgttcacaataaaaaaaaaaaacaaaacacttcttcgaacaacaacaacaacaaaaaa

SEQ ID NO: 51 - DNA, 63 bp, Artificial Sequence
  Description of Artificial Sequence Synthetic oligonucleotide
atcatttagatggtgtgggtttgtatgcaggagggttgggaagtaactaagggtgagttaatg

SEQ ID NO: 52 - DNA, 143 bp, Artificial Sequence
  Description of Artificial Sequence Synthetic polynucleotide
acgatcgcttgagcccgggagtcaaggctgcagccaactgtgatcacaccactgcactccagcctgagaaaaaccccatctcaaaaacaaatatatgtggacaaagatgatcaaatataaaaggaaaaaagacaccatgatgg

SEQ ID NO: 53 - DNA, 146 bp, Artificial Sequence
  Description of Artificial Sequence Synthetic polynucleotide
tgaatgatgtctggaacttcaaggtaggtccccaggacccctgttgttgcctgctcctctgctttggccctcatcctgctctcaggaactgctgttgcggggttgcccatggcagcatgaggtaccagctcccacctgagggctct

SEQ ID NO: 54 - DNA, 104 bp, Artificial Sequence
  Description of Artificial Sequence Synthetic polynucleotide
ccttaattttgacttagtctaatttaatcttttatggttagtactttttttgtggtctgtgtaagaaatctttgtgtctaccctaaggtcacaaagaagtttca

SEQ ID NO: 55 - DNA, 96 bp, Artificial Sequence
  Description of Artificial Sequence Synthetic oligonucleotide
actctttgccccatagtacagcggggtttgctctgattgtaggggcttcccacatcccccaggatggctgccctttgctgtggcatcactgtgtaa

SEQ ID NO: 56 - DNA, 141 bp, Artificial Sequence
  Description of Artificial Sequence Synthetic polynucleotide
acatgtatacctgatttacacagcagcagatgttcttttcaattaaacagctaggtcaatatgagggaaaaaatgaaccttcgcccctacacagaaaaaacaattccaggccaactataaacatgagatgtaagagagtgg

SEQ ID NO: 57 - DNA, 109 bp, Artificial Sequence
  Description of Artificial Sequence Synthetic polynucleotide
ggcaaaggggactccaggccactgtggggcacaggaagcagctcagtgctggagaagtcgtggggtgcagggggaagggagggaggcgaggcctggagaagagtgggct

SEQ ID NO: 58 - DNA, 104 bp, Artificial Sequence
  Description of Artificial Sequence Synthetic polynucleotide
cttttttttttttttgaaatggagtcgtgctctgtcacccaggctggagtgcagtggcacaatctcggctcactgcaacctccacctcccaggttcaagcaatt

SEQ ID NO: 59 - DNA, 122 bp, Artificial Sequence
  Description of Artificial Sequence Synthetic polynucleotide
acacagcatacgctatgcacatgtgtccacacacaccccacccacatcccacatcaccccgaccccctctgctgtccttggaaccttattacacttcgagtcactggtttgcctgtattgtg

SEQ ID NO: 60 - DNA, 134 bp, Artificial Sequence
  Description of Artificial Sequence Synthetic polynucleotide
cacctgaggtcaggagttcgagaccagcctggccaacacggtgaaaccccatctctactaaaaatacaaaaattagccaggcgtggtggtgcatgcctgtagtcccagttactcgggaggctgaggcagaagaa

SEQ ID NO: 61 - DNA, 116 bp, Artificial Sequence
  Description of Artificial Sequence Synthetic polynucleotide
gagcatatgtgcaacagtgaatgcaaaacagcttgaccaaaagtgccggctggcaggtagctggaggagaggagggctgggaggacgggagtggggccagatccaaggcacagagc

SEQ ID NO: 62 - DNA, 48 bp, Artificial Sequence
  Description of Artificial Sequence Synthetic oligonucleotide
aaatctcataatgtttttaagaaagtttgcaaatttgttaggccacat

SEQ ID NO: 63 - DNA, 62 bp, Artificial Sequence
  Description of Artificial Sequence Synthetic oligonucleotide
ggccaagccagaaaccgtggtctgctctccctccgttaaatgccattctccatcagtgaggc

SEQ ID NO: 64 - DNA, 110 bp, Artificial Sequence
  Description of Artificial Sequence Synthetic polynucleotide
tgctgtactagaacttttttttttttttgagactttagagtcaacattataattcatttcaagacatgttggaatgagttttagatttttttcccaaaacaataattaaa

SEQ ID NO: 65 - DNA, 137 bp, Artificial Sequence
  Description of Artificial Sequence Synthetic polynucleotide
aaccagtgactcgaagtgtaataaggttccaaggacagcagagggggtcggggtgatgtgggatgtgggtggggtgtgtgtggacacatgtgcatagcgtatgctgtgtgtgtgactgagcatatgtgcaacagtga

SEQ ID NO: 66 - DNA, 77 bp, Artificial Sequence
  Description of Artificial Sequence Synthetic oligonucleotide
gtcagccaccgcgcccggcctgaaaaccaatttctagagcaagcttgttcaacccatggcccacgggctgcatatgg

SEQ ID NO: 67 - DNA, 73 bp, Artificial Sequence
  Description of Artificial Sequence Synthetic oligonucleotide
gctgtgggcggtaagttgcaggggggggtttgcattttaaaaaaactacctgaaactggggatggagaggcct

SEQ ID NO: 68 - DNA, 150 bp, Artificial Sequence
  Description of Artificial Sequence Synthetic polynucleotide
ttcctgaaatagctttctttcatcaataagaattctttatattctattattcatttctaggatcaaagaaacctatagatttagtgcaatcagagaagaaaatccttaagggtatttctgtttgggtatggaagctgataaaacccactc

SEQ ID NO: 69 - DNA, 109 bp, Artificial Sequence
  Description of Artificial Sequence Synthetic polynucleotide
cacagcggccagggtcaaaggtgtcccgtggtcatctgaacaacctccctgtttccccacaccctgcccgattagtccttcctgggtggagaccctggctcggccgctg

SEQ ID NO: 70 - DNA, 155 bp, Artificial Sequence
  Description of Artificial Sequence Synthetic polynucleotide
ctcatgcctgtaatcctagcactctgggaggccaagatgggaggatcgtttgaggccaggaattaaagaccaatctggccatcatagtgagaccccgtctctattttttaaaaaacaaacaagggggccgggcgcagtggctcacgcctgtaatc

SEQ ID NO: 71 - DNA, 143 bp, Artificial Sequence
  Description of Artificial Sequence Synthetic polynucleotide
tcagcactttgggaggccgaggtgggcgaatcacttgaggtcaggagttcgagaccagcctggccaatgtggtgaaaccctgtttctactaaaaatacaaaaaaattagccgggcgtggtggcgcatgcctatattcccacct

SEQ ID NO: 72 - DNA, 126 bp, Artificial Sequence
  Description of Artificial Sequence Synthetic polynucleotide
cccagctccagctgtccctttttactgtaaaagtttcgttttgaaacaatagatatgaaaacacttggaaaaatgtaaggcattattacaaactaaatgatttatacactctctttttcttttctg

SEQ ID NO: 73 - DNA, 110 bp, Artificial Sequence
  Description of Artificial Sequence Synthetic polynucleotide
cactgaatggcattcgtgtcttgagtcttctttggatcatctcgggacacaccagtcagatgactgcatggctgtctttgggtgcggagagttgatgagcctttactcta

SEQ ID NO: 74 - DNA, 134 bp, Artificial Sequence
  Description of Artificial Sequence Synthetic polynucleotide
aggcagagataacagaataggagggcgatggtttcaatccacagggctctttggatgagaaaacatgggggtgttgttaggtagccctggtctgcttgatatttcatttctggccaggcgcagtggcacatgcc

SEQ ID NO: 75 - DNA, 39 bp, Artificial Sequence
  Description of Artificial Sequence Synthetic oligonucleotide
ctgtaatcccagctattcaggaagttggggcaagagaat

SEQ ID NO: 76 - DNA, 142 bp, Artificial Sequence
  Description of Artificial Sequence Synthetic polynucleotide
ctgctactgctatctcccatgccacaccagctgcccagaggtctgagaacctgcccacacacagggatcatcgttcccactgcaagcttctaagccagacatctggaggcccaagaatcagcccctagaacctactgactgg

SEQ ID NO: 77 - DNA, 130 bp, Artificial Sequence
  Description of Artificial Sequence Synthetic polynucleotide
tttcacatcctccccttcctaatacttgcttcttctatattccccaagcacgatgtctctggaaatgtcaaggggaccgtttatgaagccactttattttttgctctggagtcattagagttgccacctg

SEQ ID NO: 78 - DNA, 134 bp, Artificial Sequence
  Description of Artificial Sequence Synthetic polynucleotide
ggaactctatagtttttaaaatcagaaaatgtcacattggtgcaatgctataatttaacttcttttctttcttttctttttttttttttttttttgagatggagtttcgctcttgttgcccaggctggagtgca

SEQ ID NO: 79 - DNA, 162 bp, Artificial Sequence
  Description of Artificial Sequence Synthetic polynucleotide
gtggtgccatctcggctcactgcaacctccacctcccaggttcaatcgattctcctgcctcagcctcccgagtagctgggattataggcacccgccaccatgcccggctaatttttatattttttagtagagatggggtttcaccatattggccaggctggt

SEQ ID NO: 80 - DNA, 21 bp, Artificial Sequence
  Description of Artificial Sequence Synthetic primer
tggcactgctttccagcatgg

SEQ ID NO: 81 - DNA, 22 bp, Artificial Sequence
  Description of Artificial Sequence Synthetic primer
ctccccaccagaccatgagagg

SEQ ID NO: 82 - DNA, 21 bp, Artificial Sequence
  Description of Artificial Sequence Synthetic primer
atcactgggcagcatgtggca

SEQ ID NO: 83 - DNA, 22 bp, Artificial Sequence
  Description of Artificial Sequence Synthetic primer
cctgaggttcagagccatggac

SEQ ID NO: 84 - DNA, 22 bp, Artificial Sequence
  Description of Artificial Sequence Synthetic primer
catgcgaagccacactgacgtg

SEQ ID NO: 85 - DNA, 20 bp, Artificial Sequence
  Description of Artificial Sequence Synthetic primer
gcatgtgaggatcctggctc

SEQ ID NO: 86 - DNA, 20 bp, Artificial Sequence
  Description of Artificial Sequence Synthetic primer
gatctgtccctcacagcagg

SEQ ID NO: 87 - DNA, 22 bp, Artificial Sequence
  Description of Artificial Sequence Synthetic primer
ggtgtcaggaaaatgctggctg

SEQ ID NO: 88 - DNA, 20 bp, Artificial Sequence
  Description of Artificial Sequence Synthetic primer
ccaccaatggaaaaggttca

SEQ ID NO: 89 - DNA, 20 bp, Artificial Sequence
  Description of Artificial Sequence Synthetic primer
ctgtcatttctccccaccac

SEQ ID NO: 90 - DNA, 20 bp, Artificial Sequence
  Description of Artificial Sequence Synthetic primer
ggcacagcctctatggagaa

SEQ ID NO: 91 - DNA, 30 bp, Homo sapiens
cagtgaggtaaatcaggcaggcctttgccc

SEQ ID NO: 92 - DNA, 30 bp, Homo sapiens
tggggaaccatcatgctgttctccatagag

SEQ ID NO: 93 - DNA, 37 bp, Homo sapiens
ttcctgatgcgcgaacaaatgatgaaacaagccgaca

SEQ ID NO: 94 - DNA, 37 bp, Homo sapiens
tccatagcatataggtagttgatgggatagaccagca

SEQ ID NO: 95 - DNA, 37 bp, Homo sapiens
actctagcatataggtagttggtggagtggaacagaa

SEQ ID NO: 96 - DNA, 37 bp, Homo sapiens
tccccggtgcgcggacagtcgacagaacagaccaaca

SEQ ID NO: 97 - DNA, 37 bp, Homo sapiens
ttcctgatgcgcgaaccaatgatgaaacaagccgaca

SEQ ID NO: 98 - DNA, 37 bp, Homo sapiens
ttcctgatgcgcgaacaaatcatgaaacaagctgaca

SEQ ID NO: 99 - DNA, 37 bp, Homo sapiens
actctagcatataggtagttggtggagtggaacagat

SEQ ID NO: 100 - DNA, 36 bp, Artificial Sequence
  Description of Artificial Sequence Synthetic oligonucleotide
ccttccctggaactgggaatagtgggtgagatagtg

SEQ ID NO: 101 - DNA, 432 bp, Artificial Sequence
  Description of Artificial Sequence Synthetic polynucleotide
atggcaaagcaaccttctgatgtaagttctgagtgtgaccgagaaggtagacaattgcagcctgcggagaggcctccccagctcagacctggggcccctacctccctacagacagagccacaagacaggagcccagcacccatgagttgtgacaaatcaacacaaaccccaagtcctccttgccaggccttcaaccactatctcagtgcaatggtagtcatcctagaggatataggtgatctttcactgtgctttggatttatatttactggcttagatttgtatggccaccaccatagtcaagatacagaacaactcaaccacaaggatttctcatgatacctttttatagccacagccacctctctccctcttccttgagcattttgtcatatggtcattggtgattaaataaaatgtattttaatattg

SEQ ID NO: 102 - protein, 112 aa, Artificial Sequence
  Description of Artificial Sequence Synthetic polypeptide
MAKQPSDVSSECDREGRQLQPAERPPQLRPGAPTSLQTEPQDRSPAPMSCDKSTQTPSPPCQAFNHYLSAMVVILEDIGDLSLCFGFIFTGLDLYGHHHSQDTEQLNHKDFS